(19) United States
(12) Patent Application Publication
(10) Pub. No.: US 2026/0306717 A1
(43) Pub. Date: Oct. 8, 2026

(54) BIO-ORTHOGONAL HYDROGELS FOR SUSTAINED REPLEASE DRUG DELIVERY

(71) Applicant: OCULAR THERAPEUTIX, INC., Bedford, MA (US)

(72) Inventors: Fadi HASO, Bedford, MA (US); Peter JARRETT, Bedford, MA (US); Rami EL-HAYEK, Bedford, MA (US); Caroline MILLS, Bedford, MA (US)

(21) Appl. No.: 19/473,348

(22) PCT Filed: Apr. 9, 2024
(86) PCT No.: PCT/US2024/023712

§ 371 Date: Oct. 7, 2025

Related U.S. Application Data
(60) Provisional application No. 63/458,572, filed on Apr. 11, 2023.

(51) Int. Cl. A61L 27/52; A61L 27/54

(52) U.S. Cl. CPC A61L 27/52; A61L 27/54; A61L 2400/06

(57) ABSTRACT

The present invention relates to bio-orthogonal, biodegradable hydrogels for sustained drug delivery, a method for the preparation of bio-orthogonal, biodegradable hydrogels, uses thereof as an implantable material in the treatment of diseases, and a kit for preparing the hydrogels in-situ at a treatment site, and methods of treatment of using the hydrogel or kit, such as for treating ocular diseases.

### TECHNICAL FIELD

The present invention relates to bio-orthogonal, biodegradable hydrogels for sustained drug delivery, a method for the preparation of bio-orthogonal, biodegradable hydrogels, uses thereof as an implantable material in the treatment of diseases, a kit for preparing the hydrogels in-situ at a treatment site, and methods of treatment of using the hydrogel or kit.

### BACKGROUND

Hydrogels that are biocompatible and/or biodegradable are widely used in the pharmaceutical and biomedical research due to their vast application as carriers for drug delivery and scaffolds for tissue engineering. Their highly crosslinked structure as well as their ability to retain large amounts of water while maintaining their 3D structure makes them ideal candidates for localized sustained release drug delivery.

Synthetic biodegradable polymers such as polyethylene glycol (PEG), poly(N-isopropylacrylamide) (PNIPAM) and poly(vinyl alcohol) (PVA) in addition to naturally occurring polymers and polysaccharides such as agarose, collagen and fibrin are the most common types of polymer hydrogels.

While reversible physical interactions such as hydrogen bonding, ion association, and hydrophobic interaction can cause gelation through the formation of weak crosslinks, strong chemical bonds between polymer fractions are preferred as it provides desired mechanical properties. These chemical crosslinks can be achieved through multiple reactions such as redox reactions and photopolymerization, Michael addition, Schiff base formation, epoxide coupling, disulfide exchange and others. For example, EP 3 613 413 A1 describes xerogels and hydrogels formed by chemical crosslinking of polymer precursors having hydroxysuccinimide and amino (NHS/NH₂) functionality in a nucleophile-electrophile crosslinking reaction and their use for drug-delivery. However, biomolecules as active agents to be delivered by these hydrogels, such as peptides and proteins, often have terminal amine or other functional groups that interfere with the NHS/NHS functional groups and crosslinking reactions, making in-situ gel formation with such agents difficult, and preventing the use of bio-orthogonal gels for drug-delivery of biomolecules. There is thus a need for bio-orthogonal drug delivery systems that avoid such reactivity problems.

All references disclosed herein are hereby incorporated by reference in their entireties for all purposes.

### OBJECTS AND SUMMARY OF THE INVENTION

It is an object of certain embodiments of the invention, and an aspect, to provide a pharmaceutically acceptable drug-delivery system such as an implant for sustained release of an active agent to the body of a patient.

It is a further object of certain embodiments of the invention, and an aspect, to provide a drug-delivery system such as an implant that is biodegradable, e.g., that is degraded in-vivo.

It is a further object of certain embodiments of the invention, and an aspect, to provide a biodegradable drug-delivery system such as an implant or insert, for sustained release of an active agent to a patient in need thereof.

It is a further object of certain embodiments of the invention, and an aspect, to provide a bio-orthogonal drug-delivery system for sustained release of an active agent for in-situ formation of an implant or insert at a treatment site within a living body.

In further objects and aspects of certain embodiments of the invention, the active agent may be a small molecule active agent or a biomolecule active agent, e.g., a peptide, protein, virus and the like.

It is a further object of certain embodiments of the invention, and an aspect, to provide a method for manufacturing such drug-delivery systems.

It is a further object of certain embodiments of the invention, and an aspect, to provide methods for treating a disease/medical condition of a patient with a sustained release, bio-orthogonal drug-delivery system.

It is a further object of certain embodiments of the invention, and an aspect, to provide a kit comprising one or more sustained release biodegradable drug-delivery systems.

In certain embodiments, the present invention is directed to a sustained release drug-delivery system, comprising a bio-orthogonal hydrogel and at least one active agent, the bio-orthogonal hydrogel comprising a biodegradable, covalently crosslinked polymer network and containing the at least one active agent, wherein the polymer network comprises a plurality of polymer units, chemical bonds that can be cleaved by hydrolysis, wherein at least one of the hydrolysable bonds is an ester bond, and crosslinks formed by click chemistry reactions.

In certain embodiments, the polymer network forming the hydrogel comprises at least one multi-arm unit having a core unit and three or more arms connected thereto, for example 3 to 10, or 4 to 8, or 4 to 6 arms, wherein a crosslink formed by click chemistry reaction is located on the arms, preferably at the polymer unit terminus of each arm. The polymer network can comprise first and second multi-arm units, wherein the crosslinks formed by click chemistry reactions are between the arms of the first multi-arm units and the second multi-arm units. The first multi-arm units and the second multi-arm units can have the same amount of arms or a different amount of arms, and the first multi-arm units and the second multi-arm units can have the same core unit or a different core unit. In other embodiments, the first multi-arm units and the second multi-arm units can have the same amount of arms or a different amount of arms, or the first multi-arm units and the second multi-arm units can have the same core unit or a different core unit.

In certain aspects, at least one of the multi-arm units is a polymer multi-arm unit having polymer arms comprising the polymer units. The polymer units can be selected from at least one of polyethylene glycol (PEG), polyethylene oxide, polypropylene oxide, polypropylene glycol (PPG), polyvinyl alcohol (PVA), poly(vinylpyrrolidinone) (PVP), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins. In certain aspects, the polymer units in the hydrogel are polyethylene glycol units.

In certain embodiments, the hydrolysable ester bond can be part of a linker unit being located between the terminus of an arm of a multi-arm unit and the crosslinks formed by click chemistry reactions. The first multi-arm unit and/or the second multi-arm unit may include linker units comprising the hydrolysable ester bond, wherein the linker unit L can be the same or different in the first and second multi-arm unit. The linker unit L can be derived from or comprise a diacid, or an acid and amide group such as succinic, glutaric, adipic, azelaic diacids or acid-amides, or glutaramide.

In certain embodiments, in the hydrogel of the sustained release drug-delivery system, crosslinks are formed by a click chemistry reaction such as a copper-free SPAAC or IEDDA reaction, involving reacting a first functional group of a first multi-arm precursor or unit with a complementary, second functional group of a second multi-arm precursor or unit, wherein the second, complementary functional group is capable of reacting with the first functional group in a click chemistry reaction, the first and second functional groups being selected from the pairs comprising an alkyne moiety such as a dibenzocyclooctyne (DBCO) or a bicyclo[6.1.0]-nonyne (BCN), and an azide; or a norbornene or trans-cyclooctene (TCO); and a tetrazine (Tz) moiety, or functionalized derivatives thereof. In certain embodiments, the crosslinks in the hydrogel formed by click chemistry reactions comprise a chemical group selected from triazole and dihydropyridazine.

In certain embodiments of the sustained release drug-delivery system of the present invention, the active agent is selected from the group consisting of therapeutically or diagnostically active agents. The active agent is dispersed in the hydrogel and can be selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, protein binders such as nanobodies, affibodies, ankyrins, DARPins, etc., or any combinations thereof. In one embodiment, the active agent is a small molecule active agent or a biomolecule active agent, preferably a small molecule active agent, a peptide, a protein or a virus; particularly preferred a small molecule active agent, a peptide, or a protein. In a preferred embodiment, the active agent is Bevacizumab. The active agent may not be a cell or a stem cell. In one embodiment, the active agent may not be a biological sample, i.e. a tissue, a cell sample, a virus or a virion.

In certain embodiments, the sustained release drug-delivery system includes an active agent that is a peptide selected from the group consisting of Bevacizumab, Compstatin, APL-1, and Fc-III-4C, Beovu 15 (Brolucizumab), Zimura (Avacincaptad Pegol), Pegcetacoplan, Abicipar Pegol, Lampalizumab, Fovista, Risuteganib, AXT107, Elamipretide, THR149, ALM201, VGB3, and Largazole. In a particular preferred embodiment, the sustained release drug-delivery system includes the active agent Bevacizumab. In some of these embodiments, the peptide active agent is included in one or more of precursor compositions that form a bio-orthogonal hydrogel in-situ at a treatment site.

In certain embodiments, the present invention is further directed to a method of manufacturing the sustained release drug delivery system comprising a bio-orthogonal hydrogel and at least one active agent, wherein the method comprises the steps of preparing a first mixture containing a first multi-arm precursor having first functional groups suitable for forming a connection by click chemistry with second functional groups, and at least one active agent, providing a second mixture containing a second multi-arm precursor having second functional groups suitable for forming a connection by click chemistry with the first functional groups, combining the first and second mixtures, and allowing the combined reaction mixture to gel by forming click chemistry connections between the first and second functional groups, thereby forming a hydrogel containing the at least one active agent; wherein at least one of the first multi-arm precursor or the second multi-arm precursor is a polymer multi-arm precursor having polymer arms comprising polymer units, and wherein at least one of the first multi-arm precursor or the second multi-arm precursor includes a linker unit L comprising a hydrolysable ester bond.

In certain aspects of the method, at least one third multi-arm precursor having first or second functional groups suitable for forming click chemistry connections with either the first multi-arm precursor or the second multi-arm precursor can be added to one of the first or the second mixture. In some aspects of the method, the first and/or second mixture comprises at least one solvent. The at least one solvent can be selected from water, aqueous buffered media such as PBS buffer or saline solutions; or organic solvents or any mixtures thereof. In certain aspects, the method further comprises a step of removing the solvent or solvent mixture, for example by drying the formed gel.

In embodiments of the method, the multi-arm precursors can have a core unit and at least 3 arms, such as 3 to 10, or 4 to 8, or 4 to 6 arm, connected to the core unit, wherein the functional groups suitable for forming a connection by click chemistry are located at the terminus of the arms. The first multi-arm precursor and the second multi-arm precursor can have the same or a different amount of arms and/or have the same or a different core unit. In aspects thereof, the polymer units are selected from at least one of polyethylene glycol (PEG), polyethylene oxide, polypropylene oxide, polypropylene glycol (PPG), polyvinyl alcohol (PVA), poly(vinylpyrrolidinone) (PVP), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins. In certain embodiments, the polymer units are selected from at least one of polyethylene glycol (PEG) units, polypropylene glycol (PPG) units, polyglycolic acid (PGA), polylactic acid (PLA), or polylactic-co-glycolic acid (PLGA), random or block copolymers and/or combinations or mixtures of any of these, preferably polyethylene glycol units. In further embodiments, the covalently crosslinked polymer network comprises a plurality of hydrophobic polymer units and/or hydrophilic polymer units, wherein the hydrophobic polymer units are selected from at least one of polylactic acid (PLA), and polylactic-co-glycolic acid (PLGA) units; and/or wherein the hydrophilic polymer units are selected from at least one of polyethylene glycol units, polypropylene glycol units, or polyglycolic acid (PGA), preferably polyethylene glycol units.

In certain aspects, the hydrolysable ester bond is part of a linker unit being located between the polymer unit terminus of an arm of a multi-arm precursor and the functional group suitable for forming a connection by click chemistry. The first multi-arm precursor or the second multi-arm precursor, or both, may include linker units L comprising the hydrolysable ester bond, wherein the linker units L can be the same or different in the first and second multi-arm precursor. The linker unit L can be derived from or can comprise a diacid, or an acid and amide group such as succinic, glutaric, adipic, azelaic diacids or acid-amides, or glutaramide.

In certain embodiments of the method, the first and second functional groups suitable for forming a connection by click chemistry comprise moieties selected from the group consisting of alkyne such as a dibenzocyclooctyne (DBCO), or a bicyclo[6.1.0]-nonyne (BCN), cycloalkyne, strained or terminal alkene such as norbornene, or a trans-cyclooctene (TCO), azide or tetrazine (Tz), or functionalized derivatives thereof. The first and second functional groups suitable for forming a connection by click chemistry may comprise moieties selected from dibenzocyclooctyne and azide.

According to a further embodiment, the invention is directed to the sustained release drug-delivery system described herein for use as a medicament, or to a method of treatment comprising treating a disease or medical condition in a patient with a sustained release drug-delivery system as described herein. The treatment may be an ocular treatment, for example the treatment of an ocular disease such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy, or other medical conditions described herein further below.

In certain aspects of the treatment method, the drug delivery system or hydrogel can be formulated for direct injection at a treatment site of a patient, for example by parenteral administration, intratumoral injection, injection into the eye such as intravitreal, intracameral, subconjunctival, retrobulbar, sub-tenon, subretinal, or suprachoroidal injections. The hydrogel can be formulated for and administered by direct injection, by oral application, incorporated in gels, or incorporated in implants. In certain aspects, the drug delivery system comprises two or more different active agents, for example for use in a combination therapy involving the administration of more than one active agent.

In certain embodiments, the method of treatment involves an in-situ formation of the hydrogel of the drug-delivery system at a treatment site of a patient. The hydrogel can be formed in-situ at a treatment site of a patient by combining a first formulation comprising the first mixture of the manufacturing method described above and a second formulation comprising a second mixture and allowing the combined formulation mixture to gel in-situ at the treatment site. The first formulation and the second formulation can be combined shortly before administering the formulation mixture at the treatment site.

In certain embodiments involving an in-situ formation of the hydrogel of the drug-delivery system at a treatment site of a patient, the hydrogel can be formed in-situ by administering a suspension comprising a first mixture of the manufacturing method described above, a second mixture of the manufacturing method described above, and a hydrophobic organic liquid at the treatment site of the patient and allowing the combined formulation to gel in-situ at the treatment site. The hydrophobic organic liquid can be included in at least one of the first or second mixture and may be a biocompatible oil. In certain aspects thereof, the active agent is dissolved or dispersed in the hydrophobic organic liquid, or the active agent is the hydrophobic organic liquid or forms at least a part thereof.

In certain embodiments, the drug delivery system may be provided as a kit for forming a hydrogel including an active agent, the kit comprising a first precursor mixture and a second precursor mixture in separate containers for mixing and gel formation when needed. In an aspect thereof, the first formulation can be provided in a first syringe, and the second formulation in a second syringe, and the two formulations can be combined in a Y-type mixer before direct injection at the treatment site.

In an alternative embodiment of the treatment method, the first formulation and the second formulation are each administered at the treatment site, thereby combining the formulations at the treatment site, and allowing the combined formulations to gel in-situ at the treatment site. The first formulation can be in a first syringe, the second formulation in a second syringe, and the two formulations are injected simultaneously or successively at the treatment site.

Further embodiments of the invention are described herein below.

### BRIEF DESCRIPTION OF THE DRAWINGS

FIG. 1: HPLC analysis showing 1:1 (black) and 1:2 (blue) reaction of 4armPEG SAP with DBCO-Amine

FIG. 2: The use of magnetic beads to remove the excess of DBCO-Amine in the system.

FIG. 3: Graphical summary of the effect of the number of arms on gel time and swelling.

FIG. 4: Sustained release profiles of gold nanoparticles from DBCO/Azide gels.

FIG. 5: Summary of the SDS page results of Avastin solutions that are treated with different multi-arm PEGs.

FIG. 6: Summary of the SEC-HPLC data showing the presence of Avastin and PEG species in the non-pegylated samples.

FIG. 7: Summary of the ELISA measurements of Bevacizumab.

FIG. 8: Summary of the SPR results showing protein binding with VEGF.

FIG. 9: Effect of crosslinking density (MWc) on release kinetics.

FIG. 10: Release data of DBCO/tetrazine functionalized hydrogels.

FIG. 11: Gel time of DBCO/tetrazine/azide functionalized hydrogels.

FIG. 12: Release data of DBCO/tetrazine/azide functionalized hydrogels.

### DEFINITIONS

The term “bio-orthogonal” refers to a material or object (such as the hydrogels or organogels according to the present invention) that is formed by a chemical reaction that can occur inside of living systems substantially without interfering with native biochemical processes.

The term “biodegradable” refers to a material or object (such as the hydrogels according to the present invention) which is biocompatible and becomes degraded in-vivo, i.e., when placed in the human or animal body or in-vitro when immersed in an aqueous solution under physiological conditions such as pH 7.2-7.4 at 37° C. In the context of the present invention, as disclosed in detail herein below, the hydrogels once administered or deposited in the human or animal body slowly biodegrade and are cleared over time. In certain embodiments, biodegradation takes place at least in part via ester hydrolysis in the aqueous environment of the body. Biodegradation may take place by hydrolysis or enzymatic cleavage of the covalent connection or conjugation bonds, in linker groups and/or within the polymer arms. The hydrogels slowly disintegrate, resulting in clearance through physiological pathways. In certain embodiments, the hydrogels of the present invention are degradation stable over extended periods of time (e.g., about 1 month, 3 months or 6 months). In certain embodiments, the hydrogels only biodegrade, e.g., until after the active agent or at least a major amount (e.g., at least 50%, at least 75% or at least 90%) thereof has been released therefrom.

A “hydrogel” is a three-dimensional network of one or more hydrophilic natural or synthetic polymers (as disclosed herein) that can swell in water and hold an amount of water while maintaining or substantially maintaining its structure, e.g., due to chemical or physical cross-linking of individual polymer chains. Due to their high-water content, hydrogels are soft and flexible, which makes them remarkably similar to natural tissue. In the present invention the term “hydrogel” is used to refer both to a hydrogel in the hydrated state when it contains water (e.g. after the hydrogel has been formed in an aqueous solution, or after the hydrogel has been hydrated or (re-)hydrated once inserted into the eye or otherwise immersed into an aqueous environment) and to a hydrogel in its dry (dried/dehydrated) state, e.g., when it has been dried to a low water content of e.g. not more than 1% by weight.

The term “polymer network” describes a structure formed of polymer chains (of the same or different molecular structure and of the same or different molecular weight) that are cross-linked with each other. The types of polymers suitable for the purposes of the present invention are disclosed herein below.

The term “precursor” herein refers to those molecules or compounds that are reacted with each other and that are thus connected via crosslinks to form a polymer network and thus a hydrogel matrix. While other materials might be present in the hydrogel, such as active agents or buffers, they are not referred to as “precursors.”

The parts of the precursor molecules that are still present in a final polymer network are also called “units” herein. The “units” are thus the building blocks or constituents of a polymer network forming the hydrogel. For example, a polymer network suitable for use in the present invention may contain identical or different polyethylene glycol units as further disclosed herein.

The term “multi-arm precursors” means that the precursors are branched, i.e., non-linear and have a core unit and arms that extend from the core, wherein the core refers to a contiguous portion of a molecule joined to the arms.

Multi-arm precursors may have, e.g., 3-100 arms, with each arm having a terminus, bearing in mind that some precursors may be dendrimers or other highly branched materials such as dendrimers.

An arm on a precursor refers to a linear chain of chemical groups that connect a cross linkable group to a core. Some embodiments are precursors with between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms

In the case of a polymer multi-arm precursor, the precursor has a core unit and arms comprising polymer units extending from the core unit, with the polymer arms having functional groups suitable for crosslinking, which is often at the terminus of the arm.

The term “small molecule” refers to a low molecular weight (≤1000 Daltons) organic compound. Accordingly, a small molecule multi-arm precursor is branched, has a low molecular weight (≤1000 Daltons) and functional groups suitable for crosslinking at the arms.

The molecular weight of a polymer precursor as used for the purposes of the present invention and as disclosed herein may be determined by analytical methods known in the art. The molecular weight of polyethylene glycol may for example be determined by any method known in the art, including gel electrophoresis such as SDS-PAGE (sodium dodecyl sulphate-polyacrylamide gel electrophoresis), gel permeation chromatography (GPC), including GPC with static light scattering detectors (SLS) or dynamic light scattering (DLS) for simultaneous particle size determination, liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). In the case of the crosslinkable polymer gelators, such as polyethylene glycol, the molecular weight indicated herein is the number average molecular weight (Mn) determined by gel permeation chromatography using a polystyrene standard, according to standard methods known in the art. Typically, the materials, especially the multi-arm precursors are purchased with a specified molecular weight defined by the vendor. Suitable PEG precursors are for example available from a number of suppliers, such as Jenkem Technology and others.

The term “sustained release” for the purposes of the present invention is meant to characterize products which are formulated to make an active agent available over an extended period of time, thereby allowing a reduction in dosing frequency compared to an immediate release dosage form, such as a solution of an active agent that is topically applied onto the eye (i.e., eye drops). Other terms that may be used herein interchangeably with “sustained release” are “extended release” or “controlled release.” Within the meaning of the invention, the term “sustained release” comprises constant active agent release, tapered active agent release as well as any combination thereof such as a constant active agent followed by a tapered active agent release. Within the meaning of the invention, the term “tapered,” or “tapering” refers to a decrease of active agent release over time. Specifically, the term “sustained release” refers to release of an active agent from the drug-delivery system in a predetermined way and is in contrast to an immediate release like a bolus injection. The controlled release refers to the amount of the active agent release over the total number of days required for 100% release of the active agent in an aqueous solution under in-vitro physiological conditions such as at pH 7.2-7.4 and 37° C.

The term “extended period of time” as used herein refers to any period of time that would be considered by those of ordinary skill in the art as being extended with respect to treating a disease, and in particular refers to periods such as at least about 1 week, or at least about 1 month or longer, such as up to about 12 months, or any intermediate periods such as about 1 to about 6 months, about 2 to about 4 months, about 2 to about 3 months or about 3 to about 4 months.

A “zero order” release or “substantially zero order” release or “near zero order” release is defined as exhibiting a relatively straight line in a graphical representation of percent of the active agent released versus time. In certain embodiments of the present invention, substantially zero order release is defined as the amount of the active agent released which is proportional within 20% to elapsed time. The term “day 1” as used herein refers to a time point that immediately follows after “day 0”. Thus, whenever “day 1” is used, it refers to an already elapsed time period of one day or about 24 hours after administration of the drug-delivery system.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient. The active agent may be a small molecule active agent or a biomolecule active agent, e.g. a peptide, a protein, a virus or the like. In a preferred embodiment, the active agent may be a peptide or a protein. In a preferred embodiment, the active agent is Bevacizumab. The active agent may not be a cell or a stem cell. In one embodiment, the active agent may not be a biological sample, i.e. a tissue, a cell sample, a virus or a virion.

The active agent used according to the present invention may be an active agent for the treatment and/or prevention of an ocular disease or disorder, or a diagnostic agent such as a marker. In an embodiment of the invention, the active agent is a low water solubility active agent (i.e., having a solubility in water of less than about 1000 μg/mL or less than about 100 μg/mL). In other embodiments of the invention, the active agent is a highly water-soluble active agent (i.e., having a solubility in water of greater than about 1000 μg/mL or even greater than 10 mg/mL). This definition is not dependent on the agent being approved by a governmental agency.

For the purposes of the present invention, an active agent in all its possible forms, including free acid, free base, polymorphs or any pharmaceutically acceptable salts, anhydrates, hydrates, co-crystals, other solvates or derivatives, such as pro-drugs or conjugates, can be used. Whenever in this description or in the claims an active agent is referred to without further specification, even if not explicitly stated, it also refers to the active agent in the form of any such polymorphs, pharmaceutically acceptable salts, anhydrates, solvates (including hydrates) or derivatives thereof. With respect to the active agent, suitable solid forms include without limitation the pure substance form in any physical form known to the person of ordinary skill in the art. For example, the active agent may be in the form of particles. Particles can be amorphous or crystalline, or present a mixture of the two forms, and can be made of any size which could be without limitation classified as coarse, fine or ultrafine particles, the dimensions of which may be in particular visible to the naked eye or under the microscope and have shapes such as single grains and/or agglomerates. Particles may also be micronized. As used herein, the term “micronized” refers to small-size particles, in particular those of microscopic scale, which are without limitation reduced in particle size, by e.g., jet milling, jaw crushing, hammer milling, wet milling, precipitation in non-solvent, cryomilling (milling with liquid nitrogen or dry ice) and ball milling. An active agent can also be present in dissolved or dispersed state, e.g., within a solvent or in an aqueous medium, for example in the form of particles dispersed in an oil, or a compatible aqueous suspension which may optionally include further excipients such as a surfactant.

As used herein, the term “therapeutically effective” refers to the amount of active agent needed to produce a desired therapeutic result after administration. For example, in the context of the present invention, one desired therapeutic result would be the reduction of symptoms associated with DED, e.g., as measured by in-vivo tests known to the person of ordinary skill in the art, such as an increase of a Schirmer's tear test score, a reduction of Staining values as measured by conjunctival lissamine green staining or corneal fluorescein staining, a reduction of the eye dryness severity and/or eye dryness frequency score on a visual analogue scale (VAS), a reduction of the Ocular Surface Disease Index and/or the Standard Patient Evaluation of Eye Dryness score as well as a reduction of the best corrected visual acuity. In one embodiment, “therapeutically effective” refers to an amount of active agent in a sustained release intracanalicular insert capable of achieving a tear fluid concentration which is equivalent in terms of therapeutic effect to a cyclosporine concentration of 0.236 μg/mL (which is considered to be required for immunomodulation, Tang-Liu and Acheampong, Clin. Pharmacokinet. 44 (3), pp. 247-261) over an extended period of time and in particular over substantially the whole remaining wearing period of the insert once said tear fluid concentration is achieved.

As used herein, the values “d10”, “d50”, “d90” and “d100” refer to a value characterizing the proportion of particles in a particle size distribution meeting a certain particle size. In a given particle size distribution, 10% of the particles present a particles size of d10 or less, 50% of the particles present a particles size of d50 or less, 90% of the particles present a particles size of d90 or less, and substantially all particles present a particles size of d100 or less. The percentages may be given by different parameters known to the person of ordinary skill in the art, e.g., the percentages may be based on volume, weight, or the number of the particles. Thus, d50 may exemplarily be the volume-based, the weight-based or the number-based median particle size. For example, a volume-based d90 of 43 μm means that 90% of the particles by volume have a particle size of 43 μm or less. In certain embodiments, the d10, d50 and d90 are volume-based values. The particle size distribution PSD can be commonly measured by methods as known to the person of ordinary skill in the art and includes sieving as well as laser diffraction methods. In certain embodiments, the PSD is measured by laser diffraction in accordance with USP <429> Light Diffraction Measurement of Particle Size. In certain embodiments, the PSD is measured by laser diffraction using a Beckman Coulter LS 13 320 based on the optical model “Fraunhofer.rf780z” with an obscuration value ranging from 7 to 9%.

The term “patient” herein includes both human and animal patients. The biodegradable drug-delivery systems according to the present invention are therefore suitable for human or veterinary medicinal applications. Generally, a “subject” is a (human or animal) individual to which a drug-delivery systems according to the present invention is administered. A “patient” is a subject in need of treatment due to a particular physiological or pathological condition. A “patient” does not necessarily have a diagnosis of the particular physiological or pathological condition prior to receiving the drug-delivery system.

Room temperature means 20 to 25° C., such as 20° C.

All percentages, “%” herein, if not explicitly defined otherwise, are meant to be percent by weight (w/w, wt.-%).

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

As used herein, the singular forms “a”, “an”, and “the” include plural references unless the context clearly indicates otherwise.

The term “and/or” as used in a phrase such as “A and/or B” herein is intended to include both “A and B” and “A or B.”

Open terms such as “include,” “including,” “contain,” “containing” and the like mean “comprising.” These open-ended transitional phrases are used to introduce an open-ended list of elements, method steps, or the like that does not exclude additional, unrecited elements or method steps.

The term “up to” when used herein together with a certain value or number is meant to include the respective value or number.

The terms “from A to B,” “of from A to B,” and “of A to B” are used interchangeably herein and all refer to a range from A to B, including the upper and lower limits A and B.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

The abbreviation “PEG” when used herein means polyethylene glycol.

The abbreviation “PBS” when used herein means phosphate-buffered saline, and the number following is the pH thereof.

“TBS” stands for Tris-Buffered Saline, and the number following is the pH thereof.

### DETAILED DESCRIPTION

To overcome some of the drawbacks of prior art drug-delivery gels in terms of biocompatibility and versatility for delivering various types of drugs including biomolecules such as peptides, proteins, antibodies and the like, hydrogels formed by click chemistry reaction have been developed in embodiments of the present invention. In certain embodiments, the present invention is directed to hydrogel comprising an active agent, including biomolecules for delivery of the active agent to a treatment site in the patient, where the hydrogel releases the active agent over time and biodegrades before it is cleared from the body. The hydrogel comprises a biodegradable, covalently crosslinked polymer network and includes at least one active agent. The term “including an active agent” means that the hydrogel contains the active agent without the functional groups of the active agent reacting with the hydrogel, or that the active agent is covalently conjugated to the hydrogel. In one embodiment, the active agent is contained in the hydrogel and optionally at least in part further covalently conjugated to the hydrogel. In a preferred embodiment, the active agent is contained in the hydrogel without being covalently conjugated to the hydrogel. In a particular preferred embodiment, the active agent is contained in the hydrogel without being covalently conjugated to the hydrogel via an oligonucleotide. The polymer network forming the hydrogel comprises a plurality of polymer units that are crosslinked by click chemistry reactions when the hydrogel is formed. Further, the hydrogel includes chemical bonds that can be cleaved by hydrolysis, such as ester bonds, so that the hydrogel can be degraded under physiologic conditions into smaller units that can be cleared from the body.

Click chemistry provides an avenue to obtain different types of bio-orthogonal hydrogels as it encompasses a set of reactions that join two specific precursor molecules together. Click reaction chemistry is fast, spontaneous, and selective with high yields, generating no byproduct under mild conditions in aqueous environments. Specifically, copper-free strain promoted azide alkyne cycloadditions (SPAAC) and inverse electron demand Diels-Alder reactions (IEDDA) are click chemistry reactions that can be used in embodiments of the invention for hydrogel formation and crosslinking of gel-precursors by forming triazole or dihydropyridazine covalent crosslinks. Since these crosslinks are stable bonds that are not easily biodegradable, hydrogels for use as implants and drug-delivery materials in the embodiments of this invention require the presence of further, hydrolysable bonds in the polymer network. These hydrolysable bonds may be introduced in certain embodiments of the invention by introducing linkers that form hydrolysable bonds, such as ester bonds, amide bonds, carboxamide bonds or the like, with polymer units and functional groups for crosslinking at various positions of the gel precursors or the polymer network formed upon gelation.

### Hydrogel

The drug delivery systems of the present invention in accordance with certain embodiments provide sustained release of the active agent from a hydrogel that is biodegradable and biocompatible. At least one active agent is dispersed in the hydrogel and can be released from it under physiological conditions.

The structural backbone of the hydrogel is a three-dimensional polymer network that can be formed by crosslinking polymeric precursors with each other by formation of chemical bonds. Consequently, the polymer network comprises parts of the precursor molecules that are still present after the hydrogel formation, hereinafter called “units.”

In this disclosure, these crosslinks are formed by click chemistry reactions. Suitable functional groups and click chemistry reactions are described herein in detail.

### Polymeric Units and Precursors

In certain embodiments of the disclosure, the polymer network comprises multi-arm units having a core unit X and at least (≥) 3 arms attached to a core unit. The functional groups suitable for click chemistry reactions present in the respective multi-arm precursor are converted during the hydrogel formation into the crosslinks formed by click chemistry reactions of the polymer network. These crosslinks are located on the polymer arms or units, such as at the polymer unit terminus of each arm of the multi-arm unit, or at least on a majority of the multi-arm-unit termini.

The polymer network may comprise first and second multi-arm units, wherein the crosslinks formed by click chemistry reactions are between the arms of the first multi-arm units and the second multi-arm units. When the polymer network of the hydrogel comprises one or more multi-arm units, at least one of the multi-arm units has to be a polymer multi-arm unit having polymer arms comprising the polymer units of the hydrogel. Polymeric multi-arm units may also be crosslinked with non-polymeric multi-arm units such as small molecule crosslinkers.

According to certain embodiments of the invention, the hydrogel comprises a polymer network comprising at least two covalently crosslinked multi-arm precursors. In some embodiments, the hydrogel comprising a polymer network includes at least two covalently crosslinked multi-arm precursors.

Thus, a precursor is always a “functional polymer” or “functional material” such as a crosslinker (e.g., with low molecular weight) that is able to participate in the click chemistry crosslinking reaction with another precursor to form a covalently crosslinked polymer network (or matrix). Thus, the term “non-functional polymer” refers to a polymer that may be present in the hydrogel of the present invention but does not participate in the crosslinking reaction with the precursors to form a polymer network.

The term “multi-arm” precursors means that the precursors are branched, i.e., non-linear. In the case of a multi-arm polymer, a core refers to a contiguous portion of a molecule joined to arms that extend from the core, with the arms having a functional group for click chemistry, such as an azide or DBCO, which is often at the terminus of the branch. Precursors may have, e.g., 3-100 arms, with each arm having a terminus, bearing in mind that some precursors may be dendrimers or other highly branched materials such as dendrimers. An arm on a precursor refers to a linear chain of chemical groups that connect a cross linkable functional group to a polymer core. Some embodiments are precursors with between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

In certain embodiments, polymer multi-arm units present in the hydrogels are derived from one or more multi-arm PEG precursors (or of the other polymer units mentioned herein) having from 3 to 10 arms, or 4 to 8 arms, or 4, 5, 6, 7, or 8 arms. The precursors used as a first or second multi-arm unit in the hydrogels of certain embodiments of the invention may have a different or the same number of arms. In certain embodiments, the precursors used in the hydrogels of the present invention have 4 and/or 8 arms. Multi-arm precursors such as PEG precursors, Tetronic® or PLGA precursors for use in embodiments of the invention are commercially available, for example from JenKem Technology USA, SinoPEG, or Sigma-Aldrich, optionally including various functional end groups such as NHS, NH₂, azide, or DBCO.

In certain embodiments, the multi-arm precursor of the invention has a core and from 3 to 10 arms, or 4 to 8 arms, or 4, 6, or 8 arms, each arm comprising a polymer unit and having a terminus. In a preferred embodiment the multi-arm precursor has 4 or 8 arms.

In some embodiments, when each precursor is multi-arm, it comprises three or more arms and thus, three or more functional groups for click chemistry crosslinking, such that each functional group may react with another, complementary functional group of another precursor in a click chemistry reaction to form a crosslinked polymeric network of the hydrogel. Thus, for example, in some aspects, the precursor has 4 arms, wherein each arm terminates with one type of functional groups suitable for click chemistry.

According to an aspect of the invention, the hydrogel comprises at least two multi-arm precursors comprising a first multi-arm precursor comprising first functional groups and a second multi-arm precursor comprising second functional groups. In this embodiment, the first multi-arm precursor and the second multi-arm precursor are covalently cross-linked with each other in a click chemistry reaction. In this context, the multi-arm refers to at least 3 arms, 4 arms, 5 arms, 6 arms, 7 arms, or at least 8 arms, such as at least 10 arms.

The polymeric unit in the precursors and/or hydrogel can be the same or different in each arm and can be selected from at least one of polyethylene glycol (PEG), polyethylene oxide, polypropylene oxide, polypropylene glycol (PPG), poly(ethylene glycol)-block-poly(propylene glycol) copolymers, poloxamers such as Tetronic®, polyvinyl alcohol (PVA), polyvinyl acetate, poly(vinylpyrrolidinone) (PVP), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins. In certain embodiments, polymer units are selected from at least one of polyethylene glycol (PEG) units, polypropylene glycol (PPG) units, polyglycolic acid (PGA), polylactic acid (PLA), or polylactic-co-glycolic acid (PLGA), random or block copolymers and/or combinations or mixtures of any of these, preferably polyethylene glycol units. In certain embodiments of the invention, hydrophobic polymer units such as at least one poly(ethylene glycol)-block-poly(propylene glycol) copolymer are included in the polymer network, also known as Poloxamers®, such as commercially available Tetronic® poloxamers.

In certain embodiments, the polymer network comprises polylactic-co-glycolic acid (PLGA) units, or a combination of polylactic-co-glycolic acid (PLGA) units and polyethylene glycol (PEG) units, preferably copolymers of PEG and PLGA, particularly preferred block copolymers of multiarm PEG copolymerized with PLGA, or a combination of polylactic-co-glycolic acid (PLGA) units and poly(ethylene glycol)-block-poly(propylene glycol) copolymer units (Tetronic®). The ratio of polylactic-co-glycolic acid (PLGA) units to polyethylene glycol (PEG) units or poly(ethylene glycol)-block-poly(propylene glycol) copolymer units (Tetronic®) can be selected to be about 2.5:1 to about 1:2.5, or about 2:1 to about 1:2, or about 1:1.

When polylactic-co-glycolic acid (PLGA) units are used, these may have an L/G ratio (in % L or G units) ranging from 0:100 to 100:0, or 1:99 to 99:1, or 10:90 to 90:10, or 25:75 to 75:25, or 50:50. Polylactic-co-glycolic acid (PLGA) refers to a polymer with a repeating group (—(C═O)—CH(CH₃)—O)x-(—(C═O)—CH₂—O—)x, with x being at least 3. In other embodiments, x may be 1 to 2000, e.g. 1 to 1800, 1 to 1600, 1 to 1400, 1 to 1200, 1 to 1000, 1 to 800, 1 to 700, 1 to 600, 1 to 500, 1 to 400, or 1 to 300. In a particular embodiment, x may be 1 to 500, 1 to 400, or 1 to 300. In one embodiment, the polymer multi-arm units comprise 8 arms and x ranges from 1 to 300. In another embodiment, the polymer multi-arm units comprise 4 arms and x ranges from 1 to 500.

In certain embodiments, the polymeric arms comprised in the polymer network are made of or include at least one polyethylene glycol unit. Polyethylene glycol (PEG), also referred to as polyethylene oxide) refers to a polymer with a repeating group (CH₂CH₂O)n, or (CH₂CH₂O)y, respectively, with n and y being at least 3. In other embodiments, n, and y, respectively, may be 1 to 2000, e.g. 1 to 1800, 1 to 1600, 1 to 1400, 1 to 1200, 1 to 1000, 1 to 800, 1 to 700, 1 to 600, 1 to 500, 1 to 400, 1 to 300, 1 to 200, 1 to 100 or 50 to 100. In another embodiment, n, and y, respectively, may be 1 to 500, 50 to 450, 100 to 400, or 100 to 300. In one embodiment, the polymer multi-arm units comprise 8 arms and n and y, respectively, ranges from 1 to 300. In another embodiment, the polymer multi-arm units comprise 4 arms and n and y, respectively, ranges from 1 to 500.

In certain embodiments, the polymeric arms comprised in the polymer network are made of or include at least one polypropylene glycol unit. Polypropylene glycol (PPG) refers to a polymer with a repeating group (—CH₂—CH(CH₃)—O—)x, with x being at least 3. In other embodiments, x may be 1 to 2000, e.g. 1 to 1800, 1 to 1600, 1 to 1400, 1 to 1200, 1 to 1000, 1 to 800, 1 to 700, 1 to 600, 1 to 500, 1 to 400, 1 to 300, 1 to 200, 1 to 100 or 50 to 100. In a preferred embodiment, x may be 1 to 500, 50 to 450, 100 to 400, or 100 to 300. In one embodiment, the polymer multi-arm units comprise 8 arms and x ranges from 1 to 300. In another embodiment, the polymer multi-arm units comprise 4 arms and x ranges from 1 to 500.

In certain embodiments, the polymeric arms comprised in the polymer network are made of or include at least one polylactic acid unit. Polylactic acid (PLA) refers to a polymer with a repeating group (—C(C═O)—CH(CH₃)—O)x, with x being at least 3. In other embodiments, x may be 1 to 2000, e.g. 1 to 1800, 1 to 1600, 1 to 1400, 1 to 1200, 1 to 1000, 1 to 800, 1 to 700, 1 to 600, 1 to 500, 1 to 400, 1 to 300, 1 to 200, 1 to 100 or 50 to 100. In a preferred embodiment, x may be 1 to 500, 50 to 450, 100 to 400, or 100 to 300. In one embodiment, the polymer multi-arm units comprise 8 arms and x ranges from 1 to 300. In another embodiment, the polymer multi-arm units comprise 4 arms and x ranges from 1 to 500.

Each of the polymer units or arms may have an average molecular weight (Mn) in the range from about 1,000 to about 100,000 Daltons, or from about 10,000 to about 60,000 Daltons, or from about 15,000 to about 50,000 Daltons. Further, the polymer arms of the polymer multi-arm precursor may comprise polyethylene glycol (PEG) having an average molecular weight (Mn) in the range from about 1,000 to about 100,000 Daltons, or from about 10,000 to about 60,000 Daltons, or from about 15,000 to about 50,000 Daltons.

Considering the average molecular weight (Mn) of each arm, the polymeric multi-arm precursors may have in certain embodiments an average molecular weight of 5,000 to 100,000 Daltons, such as 5,000, 10,000, 15,000, 20,000 or 40,000 Daltons.

In certain embodiments, the average molecular weight (Mn) of the polymeric arms of the first multi-arm unit may be the same or different than that of the polymeric arms of the second multi-arm unit. For example, the average molecular weight of the polymeric arms of the first multi-arm unit can be higher or lower than that of the polymeric arms of the second multi-arm unit.

In certain embodiments of the invention, the polymer multi-arm units comprise a 3- to 10-arm polyethylene glycol unit, such as a 4- to 8-arm polyethylene glycol unit, or a 4-arm polyethylene glycol unit.

In certain embodiments, the polymer network comprises at least a first and a second multi-arm unit, wherein one of the first or the second multi-arm unit has a molecular weight of less than 1000 Daltons, thus being a small molecule multi-arm unit, and the other multi-arm unit is a polymer multi-arm unit having an average molecular weight (Mn) of more than 5000 Daltons.

In some embodiments, the hydrogel is composed of precursors such as multi-arm PEG precursors that each have an average molecular weight (Mn) of less than 50,000 Daltons, such as less than 45,000 Daltons, or less than 40,000 Daltons, or less than 35,000 Daltons, or less than 30,000 Daltons. When the hydrogel is degraded in-vivo, the degradation fragments obtained after cleaving the hydrolysable (ester) bonds will then have a size that allows clearance such as renal excretion from the body.

The core unit X is located the centre of a multi-arm unit from which the arms emanate. The core unit X has at least 3 connectivities c to each of which an arm or is connected, i.e., covalently bound. In certain embodiments, the core unit X has a connectivity c of 3 to 10, or 4 to 8, or 4 to 6, or 4. The core unit X may be derived from a molecule or a chemical structure having a number of c functional groups to which the arms are bound. For example, in certain embodiments the core unit X is derived from a polyol having at least 3 hydroxyl groups, or 4, 5, 6, 7, 8, 9 or 10 hydroxyl groups. In such embodiments, the polyol can be selected from glycerol, pentaerythritol, xylitol, dipentaerythritol, tripentaerythritol, hexaglycerol, isomalt, lactitol, maltitol, mannitol, or sorbitol.

In other embodiments, the core unit X is derived from polyamines such as methylene diamine, ethylene diamine, 1,3-diaminopropane, putrescine, cadaverine, hexamethylenediamine, or 1,2-diaminopropane, triamines such as diethylene triamine, propane-1,2,3-triamine, propane-1,1,3-triamine, pentane-1,3,5-triamine, or other polyamines such as triethylene tetramine, butane-1,2,3,4-tetramine, butane-1,1,4,4-tetramine.

In certain embodiments, the core unit derived from a polyol or polyamine is ethoxylated at each of its hydroxyl groups to form a multi-arm unit with the arms being polymeric PEG arms that are endcapped with the crosslinks formed by click chemistry reactions. An exemplary core unit structure derived form a polyol with three connectivities may be depicted by the following formula 1, with the connectivities c shown as OH:

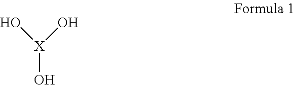

The core unit of a multi-arm unit in certain embodiments of the present invention is thus a structure appropriate to provide the desired number of arms of the precursor. For example, for 4-arm units, the core unit can be a pentaerythritol or ethylenediamine structure, whereas for 8-arm units, the core unit can be a hexaglycerol structure or a butane-tetramine structure. In certain embodiments of the invention, the core unit is pegylated at its connectivities c with polyethylene glycol arms such as in the structure depicted below. The connectivities at the end are shown as OH groups, where a linker or crosslink formed by click chemistry reactions can be connected.

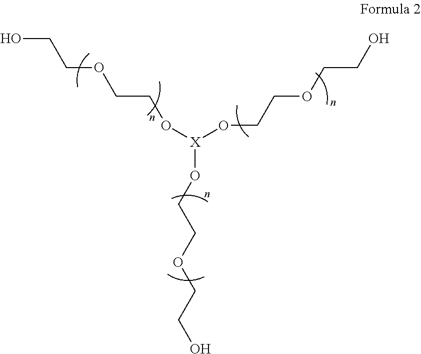

wherein n defines the number of ethoxy groups of the PEG unit, and n may be 1 to 2300. In other embodiments, n may be 1 to 2000, e.g. 1 to 1800, 1 to 1600, 1 to 1400, 1 to 1200, 1 to 1000, 1 to 800, 1 to 700, 1 to 600, 1 to 500, 1 to 400, 1 to 300, 1 to 200, 1 to 100 or 50 to 100. In another embodiment, n may be 1 to 500, 50 to 450, 100 to 400, or 100 to 300.

In certain embodiments, the polymer multi-arm units can be represented by formula (ii):

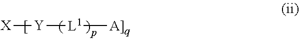

wherein X is the core unit, Y is a polymeric unit such as PEG, L is a linker unit comprising the hydrolysable ester bond, A is a crosslink formed by a click chemistry reaction, p is either 0 or 1, and q is an integer from 2 to 10, or 3 to 10.

### Linker Unit

The inclusion of linker groups that are labile to hydrolysis (hydrolysable bonds) into the polymer network allows a biodegradation of the hydrogel under physiological conditions. By cleaving hydrolysable bonds, the hydrogel can be degraded into smaller constitutional units having low molecular weight and can be cleared from the body by usual physiological pathways.

The polymer network of the hydrogel according to certain embodiments of the present invention comprises chemical bonds that can be cleaved by hydrolysis, wherein at least one of the hydrolysable bonds is an ester bond. In certain embodiments, this hydrolysable ester bond is provided by a linker unit L being located between the terminus of an arm of a multi-arm unit and the crosslinks formed by click chemistry reactions. In an aspect, the first multi-arm unit and the second multi-arm unit include linker units L comprising the hydrolysable ester bond, wherein the linker unit L can be the same or different in the first and second multi-arm unit. In another aspect, only the first multi-arm unit or the second multi-arm unit include linker units L comprising the hydrolysable ester bond.

The linker unit L may be derived from or comprised of a diacid, or an acid and amide group such as succinic(S), glutaric (G), adipic (AP), azelaic (AZ) diacids or acid-amides, or glutaramide (GA).

In certain embodiments, the linker unit L comprises a structure represented by Formula (i):

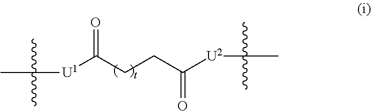

wherein U¹ and U² are independently NH or O and can be the same or different, wherein at least one of U¹ and U² is O, and wherein t is an integer from 0 to 10. In other embodiments, t is an integer from 0 to 9, from 0 to 8, from 0 to 7, from 0 to 6, from 0 to 5, from 0 to 4, from 0 to 3, from 0 to 2, or from 0 to 1. In other embodiments, t is 0, tis 1, tis 2, tis 3, tis 4, tis 5, tis 6, tis 7, tis 8, tis 9, or tis 10.

The biodegradability of the hydrogel can be tuned to have the desired stability intended for specific uses by choosing the appropriate linker unit L and/or the amount and/or location of linker units L. For example, the amount of the linker units in the hydrogel can have an influence on the stability of the corresponding hydrogel over time in aqueous environments.

In certain embodiments, solely the first or the second multi-arm precursor contains a linker unit L comprising the hydrolysable ester bond. In another embodiment, the first and the second multi-arm precursor include a linker unit L comprising the hydrolysable ester bond.

Further, the rate of biodegradation/hydrolysation of the ester bonds contained in the linker units L increases from longer chain lengths (larger t, such as 6 in azelate linker units) to shorter chain lengths (smaller t, such as 1 in succinate linker units). The shorter the linker chain length, the faster the ester bonds are hydrolysed.

In certain embodiments, the chain length (t₁) of the linker units of the first multi-arm precursor is shorter (t₁<t₂) than, longer (t₁>t₂) than, or the same (t₁=t₂) as the chain length (t₂) of the linker units of the second multi-arm precursor.

### Hydrogel Formation

Several methods for manufacturing hydrogels are known to the skilled artisan, and these methods can be principally applied and suitably adapted in embodiments of the present invention.

In an embodiment of the invention, a method for synthesizing drug delivery system is provided that includes the following steps:
a) preparing a first mixture containing a first multi-arm precursor having first functional groups suitable for forming a connection by click chemistry with second functional groups,
b) providing a second mixture containing a second multi-arm precursor having second functional groups suitable for forming a connection by click chemistry with the first functional groups,
c) combining the first and second mixtures,
d) adding at least one active agent to the first, second or combined first and second mixtures, and
e) allowing the combined reaction mixture to gel by forming click chemistry connections between the first and second functional groups, thereby forming a hydrogel containing the at least one active agent.

In this embodiment, at least one of the first multi-arm precursor or the second multi-arm precursor is a polymer multi-arm precursor having polymer arms comprising polymer units such as PEG. The first multi-arm precursors and the second multi-arm precursors used as starting materials in the method of the invention are known in the art or can be obtained as described herein.

Further, at least one of the first multi-arm precursor or the second multi-arm precursor includes a linker unit L comprising a hydrolysable ester bond. By including the linker unit, the obtained hydrogel becomes biodegradable, as described above in chapter “linker units.”.

Since the above described multi-arm units originate from the respective multi-arm precursors, the parts of the multi-arm precursor that do not participate in the crosslinking reaction, i.e., the linker, the polymer arms, and the core unit, remain unchanged and are accordingly as defined above for the multi-arm units.

In some embodiments, the active agent is added to both the first and second mixture. In other embodiments, the active agent is added to the combined mixture of first and second mixture. Each of the first or second mixture may include a solvent, for example water, or an aqueous solvent such as an aqueous buffer as described herein. Each of the first or second mixture may include additional ingredients such as solvent, pharmaceutical excipients, buffer, additional therapeutic or diagnostic agents, thickeners, viscosity enhancers, stabilizers, adjuvants, and the like. The second mixture may further include one or more active agents.

In certain embodiments, the first and/or second mixture comprises at least one solvent. The at least one solvent can be selected from acetone, acetonitrile, benzyl alcohol, chloroform, dichloromethane (DCM), dioxane, dimethyl carbonate (DMC), dimethyl sulfoxide (DMSO), methanol, ethanol or propanol, ethyl acetate, ethyl formate, ethyl propionate, glycofurol, hexafluoro-isopropanol, isosorbide dimethyl ether, isopropanol, methyl chloride, methylene chloride, methyl ethyl ketone, N-methylpyrrolidone, propylene carbonate, triethylamine, or tetrahydrofuran; water, aqueous buffered media such as PBS buffer or saline solutions; or any mixtures thereof. In certain embodiments, such as for in-situ preparation of the drug-delivery system, the at least one solvent can be selected from water, aqueous buffered media such as PBS buffer or saline solutions; or any mixtures thereof, optionally admixtured with biocompatible organic solvents.

The method of certain embodiments can further comprise a step of removing the solvent or solvent mixture. Removal of the solvent can be done by any suitable drying method, and may be performed by one of hot air convection, drying in inert gas atmosphere or streams of inert gases such as nitrogen or argon, or direct drying, indirect or contact drying, spray drying, dielectric drying, vacuum drying, freeze drying, supercritical or superheated steam drying, or combinations of any of these.

In one embodiment when the first and second mixtures have been combined to a combined reaction mixture, the precursors react in a click chemistry reaction to form a covalently cross-linked hydrogel polymer network. The reaction may be initiated or promoted by heating, or can occur at ambient conditions, even in-vivo.

The hydrogel formed may be shaped, and shaping may comprise molding or extruding or casting the reaction mixture prior to complete gelling of the hydrogel, then allowing the mixture to gel, and optionally drying, complete or at least partial removal of the solvent, to obtain a xerogel. Molding may be done by filling the reaction mixture into a mold or tubing prior to complete gelling of the hydrogel, allowing the mixture to gel, and optionally drying. In some embodiments the reaction mixture may be filled into a fine diameter tubing or needle in order to prepare a hydrogel strand that may be used directly or dried prior to use. The reaction mixture may also be applied as a coating on a substrate. As part of the process, the crosslinked hydrogel can be deformed and rigidified to allow injection through a needle lumen, the rigidity and reshaping being reversible upon contacting the warmth and/or moisture of tissue. Rigidity could be provided through crystallization, a secondary crosslink mechanism, or a water soluble temporary structural component, e.g., PEG fibers.

A combined reaction mixture including the precursors mixed therein can be made with a viscosity suitable for introduction through a small gauge needle using manual force. A small gauge needle has a diameter less than the diameter of a needle with a gauge of 27, e.g., 28, 29, 30, 31, 32, or 33 gauge, with the gauge being specific for inner and/or outer diameters. Moreover, hollow-tube wires, as used in the intravascular arts, may be used to deliver the materials to an implantation site for forming the drug-delivery device in-situ, including those with inner and/or outer diameters equivalent to the small gauge needles, or smaller. Thus, a viscosity of between about 1 to about 100,000 mPa·s may be used; artisans will immediately appreciate that all the ranges and values within the explicitly stated ranges are contemplated e.g., about 10 to about 10,000 mPa·s, less than about 5 to about 10,000 mPa·s, less than about 100 or about 500 mPa·s, or between about 1 and about 100 mPa·s. The viscosity may be controlled, e.g., by choosing appropriate precursors, adjusting solids and/or solvent concentrations, and reaction kinetics. In general, lower concentrations of precursors and/or lower molecular weights favor a lower viscosity.

Viscosity enhancers may be used in conjunction with precursors. In certain embodiments, the viscosity enhancers do not react with the precursors to form covalent bonds. While it is appreciated that precursors that are generally free of such bonding may sometimes participate in unwanted side reactions, these have little effect on the hydrogel so that the precursors are “free” of such reactions. For instance, while the precursors react by click chemistry reactions, the viscosity enhancers may be free of functional groups reactive in click chemistry that can form covalent bonds with functional groups of the precursors, even if there is some low level of unwanted side reactions. Viscosity enhancers may be hydrophilic polymers with a molecular weight, e.g., of at least 20,000, or from about 10,000 to about 500,000 Daltons; artisans will immediately appreciate that all values and ranges between these explicitly stated values are described, e.g., at least about 100,000 or 200,000. A concentration of about 1% to about 40%, or about 5% to about 25% w/w may be used, for instance. PEG (e.g., M.W. 100,000 to 250,000) is useful, for example. Viscosity enhancers may include one or more biodegradable links as described herein for precursors. Viscosity enhancers can be useful to prevent precursors from running-off a tissue site before the precursors crosslink to form a gel.

The provided method is not limited to two multi-arm precursors and can further comprise the addition of at least one third multi-arm precursor having first or second functional groups suitable for forming click chemistry connections with either the first multi-arm precursor or the second multi-arm precursor in one of steps a) to c).

In one embodiment, in the method of the invention, the first and second functional groups suitable for forming a connection by click chemistry comprise moieties are selected from the group consisting of alkyne such as a dibenzocyclooctyne (DBCO), or a bicyclo[6.1.0]-nonyne (BCN), cycloalkyne, strained or terminal alkene such as norbornene, or a trans-cyclooctene (TCO), azide or tetrazine (Tz).

In a preferred embodiment of the method of the invention, the first and second functional groups suitable for forming a connection by click chemistry comprise moieties selected from dibenzocyclooctyne and azide.

In one embodiment of the method of the invention, the first multi-arm precursor comprising azide (N₃) moieties is selected from the group consisting of:

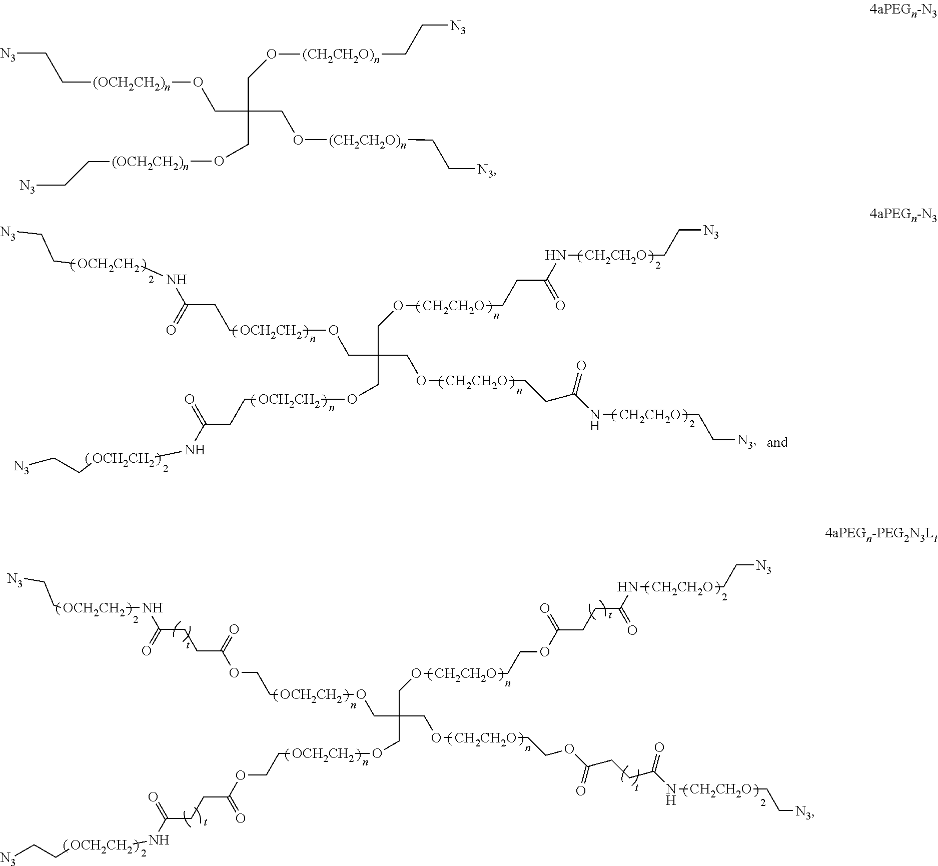

Wherein L is a linker, n is 1 to 2300, and t is an integer from 0 to 10, and/or the corresponding structures with 6 arm, or 8 arm PEG cores thereof.

In one embodiment of the method of the invention, the first multi-arm precursor 5 comprising azide (N₃) moieties is selected from the group consisting of 8a5kPEG-N₃, 8a10kPEG-N₃, 8a15kPEG-N₃, 8a20kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ.

In one embodiment of the method of the invention, the second multi-arm precursor comprising dibenzocyclooctyne (DBCO) moieties is selected from the group consisting of:

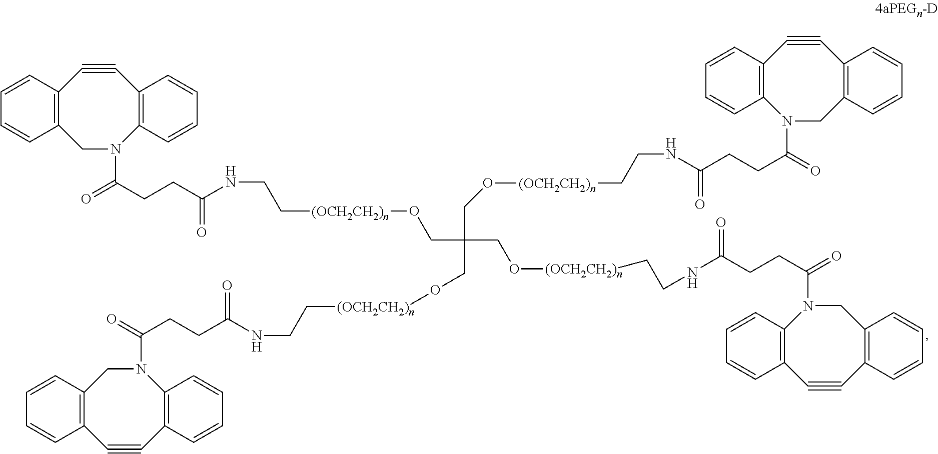

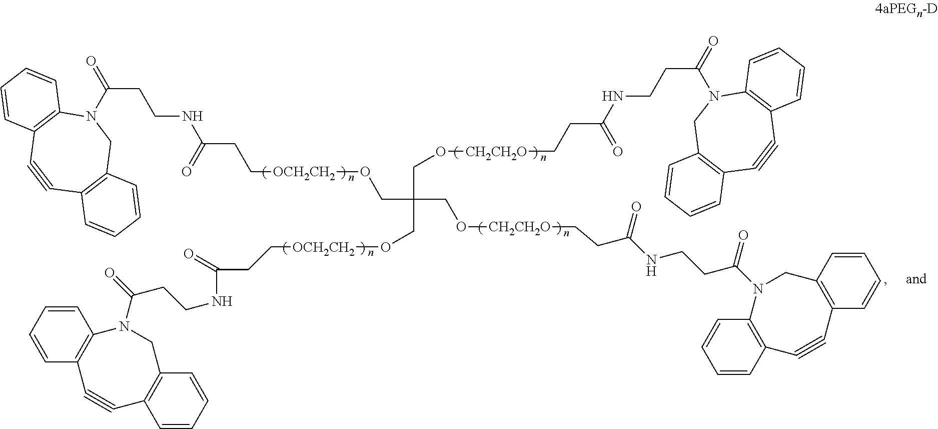

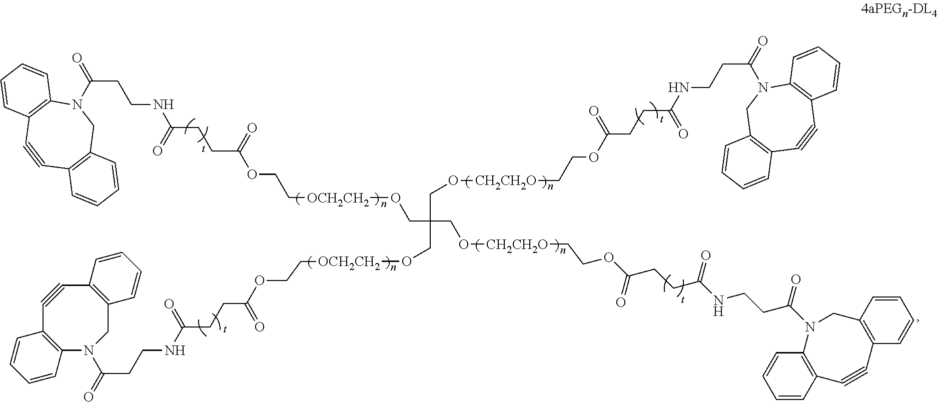

wherein L is a linker, n is 1 to 2300, and t is an integer from 0 to 10, and/or the corresponding structures with 6 arm, or 8 arm PEG cores thereof.

In one embodiment of the method of the invention, the second multi-arm precursor comprising dibenzocyclooctyne (DBCO) moieties is selected from the group consisting of 4a20kPEG-D, 6a20kPEG-D, 8a20kPEG-D, 4a20kPEG-DS, 8a10kPEG-DG, 8a15kPEG-DG, 4a20kPEG-DG, 4a20kPEG-DAP, 4a20kPEG-DAZ, 4a20kPEG-DPEG₂₃AZ, and 4a20kPEG-sDG.

In one embodiment of the method of the invention, the first multi-arm precursor comprising azide (N₃) moieties and the second multi-arm precursor comprising dibenzocyclooctyne (DBCO) moieties are selected from the group consisting of:

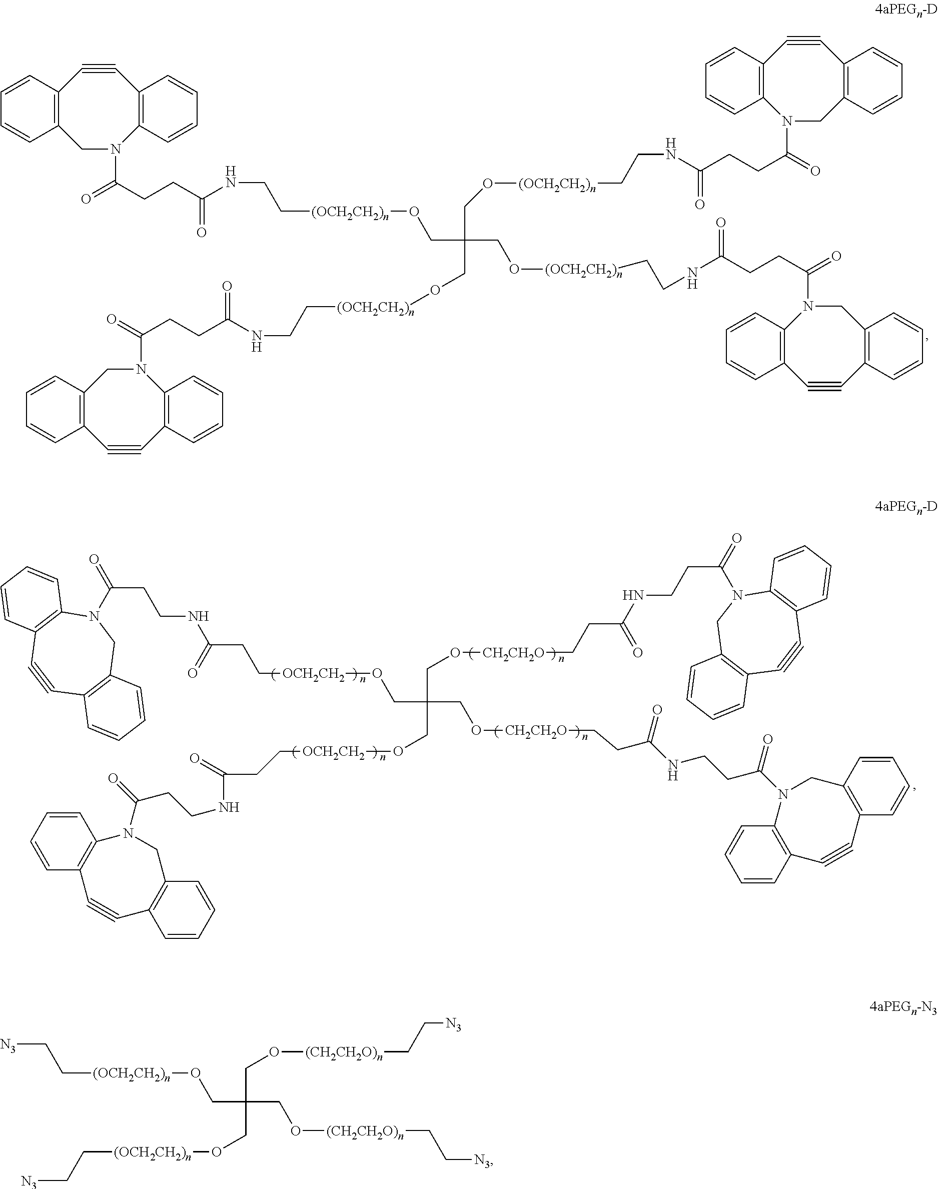

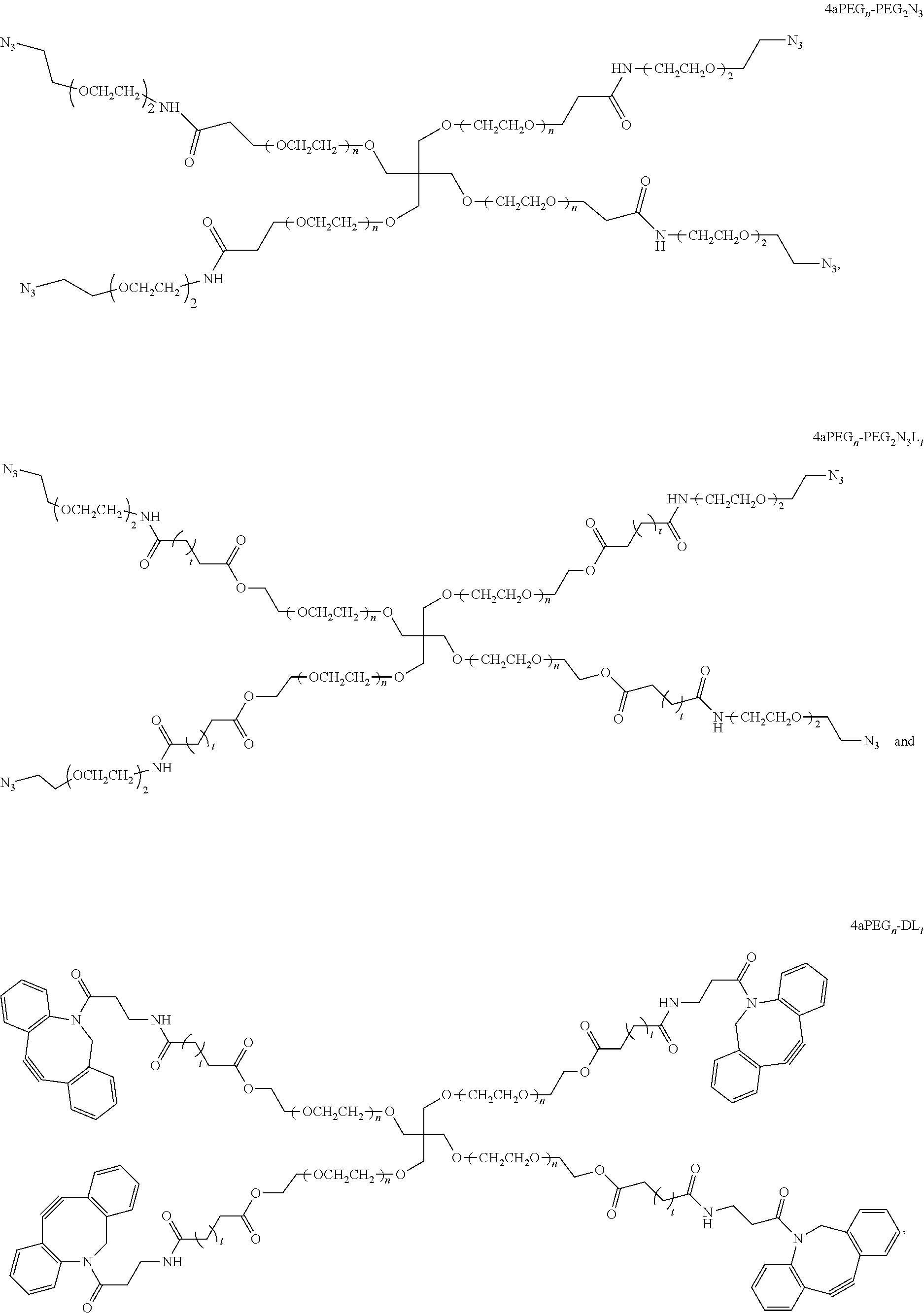

wherein L is a linker, n is 1 to 2300, and t is an integer from 0 to 10, and/or the corresponding structures with 6 arm, or 8 arm PEG cores thereof.

In one embodiment of the method of the invention, the first multi-arm precursor comprising azide (N₃) moieties is selected from the group consisting of 8a5kPEG-N₃, 8a10kPEG-N₃, 8a15kPEG-N₃, 8a20kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ; and the second multi-arm precursors comprising DBCO moieties is selected from the group consisting of 4a20kPEG-D, 6a20kPEG-D, 8a20kPEG-D, 4a20kPEG-DS, 8a10kPEG-DG, 8a15kPEG-DG, 4a20kPEG-DG, 4a20kPEG-DAP, 4a20kPEG-DAZ, 4a20kPEG-DPEG₂₃AZ, 4a20kPEG-sDG and 4a20kPEG-PEG₂BG.

In one embodiment of the method of the invention, wherein the first multi-arm precursor comprising azide (N₃) moieties and the second multi-arm precursor comprising dibenzocyclooctyne (DBCO) moieties are selected from the group consisting of:

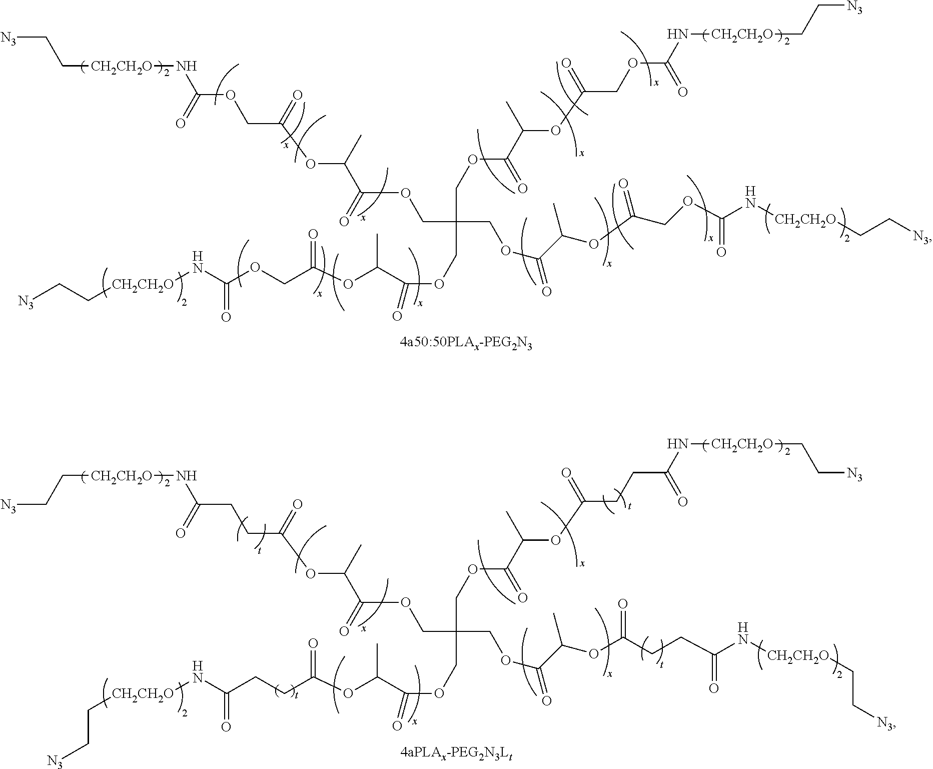

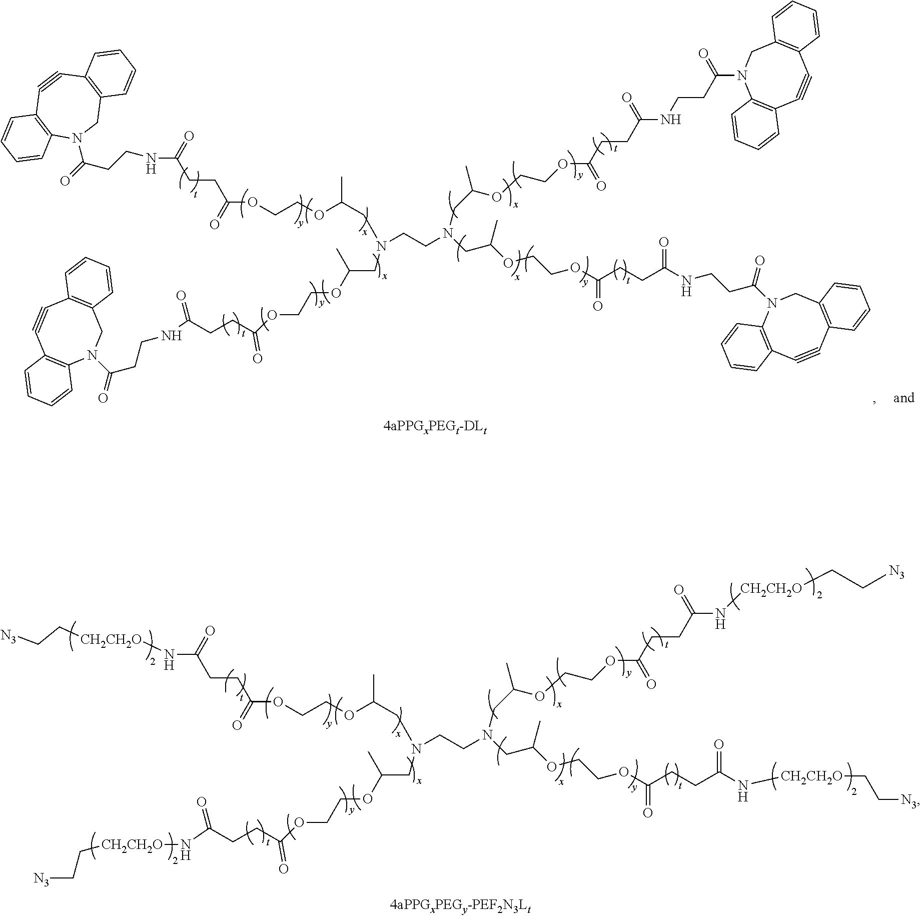

wherein x, y defining the number of PPG, PEG, PLA or PGA units and t is an integer 5 from 0 to 10, and/or the corresponding structures with 6 arm, or 8 arm PEG cores thereof.

In one embodiment of the method of the invention, the first multi-arm precursor comprises tetrazine (TET) moieties selected from the group consisting of 8a5kPEG-TET, 8a10kPEG-TET, 8a15kPEG-TET, 8a20kPEG-TET, 8a20kPEG-PEG₅TET, 4a20kPEG-TET, or ester linked 4a20kPEG-PEG₅TETS, 4a20kPEG-PEG₅TETG, 4a20kPEG-PEG₅TETAP, and 4aPEG-PEG₅TETAZ; and the second multi-arm precursors comprises DBCO moieties selected 10 from the group consisting of 4a20kPEG-D, 6a20kPEG-D, 8a20kPEG-D, 4a20kPEG-DS, 8a10kPEG-DG, 8a15kPEG-DG, 4a20kPEG-DG, 4a20kPEG-DAP, 4a20kPEG-DAZ, 4a20kPEG-DPEG₂₃AZ, 4a20kPEG-sDG and 4a20kPEG-PEG₂BG.

In one embodiment of the method of the invention, the first multi-arm precursor comprises tetrazine (TET) moieties selected from the group consisting of 8a5kPEG-TET, 8a10kPEG-TET, 8a15kPEG-TET, 8a20kPEG-TET, 8a20kPEG-PEG₅TET, 4a20kPEG-TET, or ester linked 4a20kPEG-PEG₅TETS, 4a20kPEG-PEG₅TETG, 4a20kPEG-PEG₅TETAP, and 4aPEG-PEG₅TETAZ; and the second multi-arm precursors comprises a norbornene (Nor) moiety selected from the group consisting of 4a20kPEG-Nor, 6a20kPEG-Nor, 8a20kPEG-Nor, 4a20kPEG-NorS, 8a10kPEG-NorG, 8a15kPEG-NorG, 4a20kPEG-NorG, 4a20kPEG-NorAP, 4a20kPEG-NorAZ, 4a20kPEG-NorPEG₂₃AZ, and 4a20kPEG-PEG₂NorG.

In one embodiment of the method of the invention, the first multi-arm precursor comprising azide (N₃) moieties is selected from the group consisting of 8a5kPEG-N₃, 8a10kPEG-N₃, 8a15kPEG-N₃, 8a20kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ; and the second multi-arm precursors comprising bicyclononyne (BCN) moieties is selected from the group consisting of 4a20kPEG-D, 6a20kPEG-BCN, 8a20kPEG-BCN, 4a20kPEG-BCNS, 8a10kPEG-BCNG, 8a15kPEG-BCNG, 4a20kPEG-BCNG, 4a20kPEG-BCNAP, 4a20kPEG-BCNAZ, 4a20kPEG-BCNPEG₂₃AZ, and 4a20kPEG-PEG₂BCNG.

In one embodiment of the method of the invention, the first multi-arm precursor comprising azide (N₃) moieties, is selected from the group consisting of 4a18Tetronic1307-PEG₂N₃, 4a20kPLA-PEG₂N₃, 8a20k50:50PLGA-PEG₂N₃, and 4a20kPLA-PEG₂N₃S.

In one embodiment of the method of the invention, the second multi-arm precursor comprising a dibenzocyclooctyne (DBCO) moieties is selected from the group consisting of 4a20kPEG-DAZ, 8a20kPEG-D and 4a18Tetronic1307-DAZ.

In one embodiment of the method of the invention, the first multi-arm precursor comprising azide (N₃) moieties is selected from the group consisting of 4a18Tetronic1307-PEG₂N₃, 4a20kPLA-PEG₂N₃, 8a20k50:50PLGA-PEG₂N₃, and 4a20kPLA-PEG₂N₃S; and the second multi-arm precursor comprising dibenzocyclooctyne (DBCO) moieties is selected from the group consisting of 4a20kPEG-DAZ, 8a20kPEG-D and 4a18Tetronic1307-DAZ.

Further first multi-arm precursors and second multi-arm precursors that are suitable as starting materials in the method of the invention, are described herein, e.g. in the chapters “Polymeric units and Precursors” and “Functional groups suitable for click chemistry reactions”.

The amount of differently functionalized PEG multi-arm precursors used for hydrogel formation can be calculated through stoichiometric equations of molar proportion and converting moles to grams, as described in principle, for example in WO 2024/035690 A1, paragraphs 132 to 134, incorporated herein by reference.

The molecular weight between crosslinks, MWc, a parameter defining the mesh size within the hydrogel, or the crosslink density, can be calculated through the sum of the average arm length of each multi-arm PEG precursor (PEG, MW=molecular weight).

$$PEG Arm Length = \frac{PEG MW}{\# arms}$$

$$MWc = Precursor 1 Arm Length + Precusor 2 Arm Length ( + Precursor \times Arm Length )$$

For example, a hydrogel from equimolar amounts of 8a15kDG and 8a20kN3 (see Example 7) has an MWc of 15 kDa/8+20 kDa/8=1,875 Da+2,500 Da=4,375 Da. Similar calculations can be done for other types of polymers as described herein.

In certain embodiments of the invention, the sustained release drug-delivery system has a MWc of at least 2,500 Da, such as at least 3,000 Da, 4,000 Da, 5,000 Da, 6,000 Da, 7,000 Da, 8,000 Da, 9,000 Da, 10,000 Da, 12,000 Da, or at least 15,000 Da; or less than 25,000 Da, such as up to 20,000 Da, or up to 18,000 Da.

### Functional Groups Suitable for Click Chemistry Reactions

The three-dimensional polymer network of the hydrogel is formed by reacting at least one first multi-arm precursor having functional groups suitable for click chemistry with at least one second multi-arm precursor having functional groups suitable for forming click chemistry connections with the functional groups of the first multi-arm precursor.

Functional groups suitable for click chemistry are such functional groups that enable click chemistry reactions such as strain promoted alkyne-azide cycloaddition (SPAAC), also termed as the Cu-free click reaction, or inverse electron demand Diels-Alder ligation (IEDDA) type click chemistry coupling reactions. An overview of such types of reaction is given in H. C. Kolb; M. G. Finn; K. B. Sharpless (2001). “Click Chemistry: Diverse Chemical Function from a Few Good Reactions”, Angewandte Chemie International Edition, 40 (11): 2004-2021), incorporated herein by reference.

Other suitable click chemistry reactions include aldehyde/ketone condensation, cyanobenzothiazole condensation; strain-promoted, oxidation-controlled cyclooctyne-1,2-quinone cycloaddition (SPOCQ); 1,3-dipolar cycloadditions, [3+2] cycloadditions such as alkene-nitrone cycloadditions or alkyne-nitrone cycloadditions, [4+2] cycloadditions; and hetero-Diels-Alder reactions.

SPAAC requires a ring-structured alkyne such as dibenzocyclooctyne (DBCO) and Bicyclo[6.1.0]nonyne (BCN) to react with an aliphatic azide. This strained chemistry causes the reaction to happen efficiently without the need of a copper catalyst required in copper (I)-catalyzed azide-alkyne click chemistry reactions (CuAAC). Similarly, IEDDA requires the reaction of norbornene or DBCO and tetrazine without the need of a catalyst. Thus, the advantage of SPAAC and IEDDA over CuAAC and electrophile-nucleophile reactions such as NHS—NH₂ is that there is no catalyst needed and no byproduct exists after the reaction is completed.

SPAAC and IEDDA coupling reactions are bioorthogonal reactions with selective and quantitative yields under mild conditions that can occur even inside of living systems without interfering with native biochemical processes. These click chemistry reactions utilize a pair of functional groups, for example cyclooctynes and azides, that exclusively and efficiently react with each other while remain inert to naturally occurring functional groups:

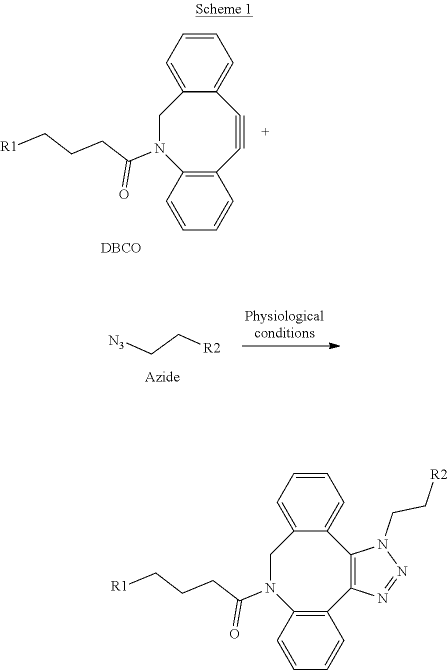

wherein R1 and R2 are polymer arms, optionally including a linker L.

This reaction is suitable for forming the covalent crosslinks of the polymer network of the hydrogel from correspondingly functionalized multi-arm precursors as described herein.

Among the large number of known cyclooctynes, the dibenzocyclooctynes (DBCO) compounds comprise a class of reagents that possesses reasonably fast kinetics in SPAAC reactions and good stability in aqueous buffers. Within physiological temperature and pH ranges, the DBCO group will not react with amines or hydroxyls that are naturally present in many biomolecules. Additionally, reaction of the DBCO group with the azide group is significantly fast.

The advantages of DBCO based SPAAC are, for example, its biocompatibility as there are no cytotoxic copper catalysts required that may remain in undesirable traces in the hydrogels. Another advantage is the use of mild reaction conditions: connecting arms of the multi-arm precursors is possible in aqueous buffered media or common organic solvents at physiological conditions. Furthermore, DBCO and azide moieties are long term stable and have a high selectivity and specificity as azide groups react only with DBCO in the presence of amine, hydroxyl, thiol, and acid groups, as well as other protein functional groups. Also, the reactions lead to the formation of a stable triazole in quantitative yield with high reaction rate, and leave no byproducts. Similar advantages are provided by IEDDA coupling reactions and other types of catalyst free click chemistry reactions mentioned herein before.

Since click chemistry reactions utilize a pair of functional groups that react exclusively and efficiently with each other, one multi-arm precursor can only possess functional groups corresponding to one part of the pair of these functional groups (e.g., either just azide or just DBCO). Otherwise, an intramolecular click chemistry reaction would take place and the multi-arm precursor would not be suitable to construct the hydrogel of the present invention comprising a polymer network.

Accordingly, a first multi-arm precursor having first functional groups suitable for click chemistry has to react with a second multi-arm precursor having second functional groups suitable for forming click chemistry connections with the first functional groups of the first multi-arm precursor.

The polymer multi-arm precursors may be represented by formula (iii):

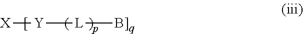

wherein X is the core unit, Y is a polymeric unit, such as PEG, L is a linker unit comprising the hydrolysable ester bond, B is a functional group suitable for forming a connection by click chemistry, p is either 0 or 1, and q is an integer from 3 to 10.

In certain embodiments, suitable functional groups (B) comprise moieties selected from the group consisting of alkyne, cycloalkyne such as a dibenzocyclooctyne (DBCO), or a bicyclo[6.1.0]nonyne (BCN), strained or terminal alkene such as norbornene, or a trans-cyclooctene (TCO), azide or tetrazine (Tz).

For example, in an exemplary embodiment, a first multi-arm precursor having functional groups comprising an azide moiety can be reacted with a second multi-arm precursor having functional groups comprising a dibenzocyclooctyne (DBCO) moiety to form the polymer network of the hydrogel of exemplary embodiments of the present invention. In one embodiment, the polymer network of the hydrogel is formed by solely reacting a first multi-arm precursor having functional groups comprising an azide (N₃) moiety with a second precursor having functional groups comprising a dibenzocyclooctyne (DBCO) moiety, i.e. no additional reaction with other multi-arm precursors containing functional groups other than an azide or a DBCO moiety is carried out to form the polymer network of the hydrogel. In one embodiment, the first multi-arm precursor having functional groups comprising an azide moiety is not derived from pentaerythritol tetrakis(2-bromoisobutyrate).

In one embodiment, the polymer network of the hydrogel is formed by reacting a first multi-arm precursor having functional groups comprising an azide (N₃) moiety with a second multi-arm precursor having functional groups comprising a dibenzocyclooctyne (DBCO) moiety, and the active agent is a small molecule active agent, a peptide, a protein or a virus.

In another embodiment, the polymer network of the hydrogel is formed by reacting a first multi-arm precursor having functional groups comprising an azide (N₃) moiety with a second multi-arm precursor having functional groups comprising a dibenzocyclooctyne (DBCO) moiety, and the active agent is contained in the hydrogel without being covalently conjugated to the hydrogel, preferably, wherein the active agent is contained in the hydrogel without being covalently conjugated to the hydrogel via an oligonucleotide, insert after claim 26; delimitation re WO2021113115, WO2013036847, E. L. Schneider et al., G. W. Ashley et al, polymer-drug conjugate

In a particular preferred embodiment, the polymer network of the hydrogel is formed by reacting a first multi-arm precursor having functional groups comprising an azide (N₃) moiety with a second multi-arm precursor having functional groups comprising a dibenzocyclooctyne (DBCO) moiety, preferably, wherein the first multi-arm precursor having functional groups comprising an azide moiety is not derived from pentaerythritol tetrakis(2-bromoisobutyrate, the active agent is a small molecule active agent, a peptide, a protein or a virus, and the active agent is contained in the hydrogel without being covalently conjugated to the hydrogel, preferably, wherein the active agent is contained in the hydrogel without being covalently conjugated to the hydrogel via an oligonucleotide.

Exemplary polymer 4 arm precursor having functional groups suitable for click chemistry and no linker units or linker units L are as follows. The abbreviated nomenclature of multi-arm precursors in this disclosure generally complies with the following formula qM_{w}Y-BL, wherein q is the number of arms, Mw is the average molecular weight of the precursor, Y is the polymeric unit, B is the functional group and L is the linker.

The following functional groups B are abbreviated as follows: dibenzocyclooctyne (D), dibenzocyclooctyne sulfo-amine (sD), azide (N₃), bicyclo[6.1.0]nonyne (B), norbornene (N), trans-cyclooctene (TCO), tetrazine (T), amine (NH₂) and N-hydroxysuccinimidyl (NHS). In general structures, where the molecular weight is not defined, Y is written as Y_{number of repeating units} and when linker units L as defined above are shown without specified t, the abbreviation L; is used.

### Exemplary Polymer 4 Arm Precursors are:

### No Linker:

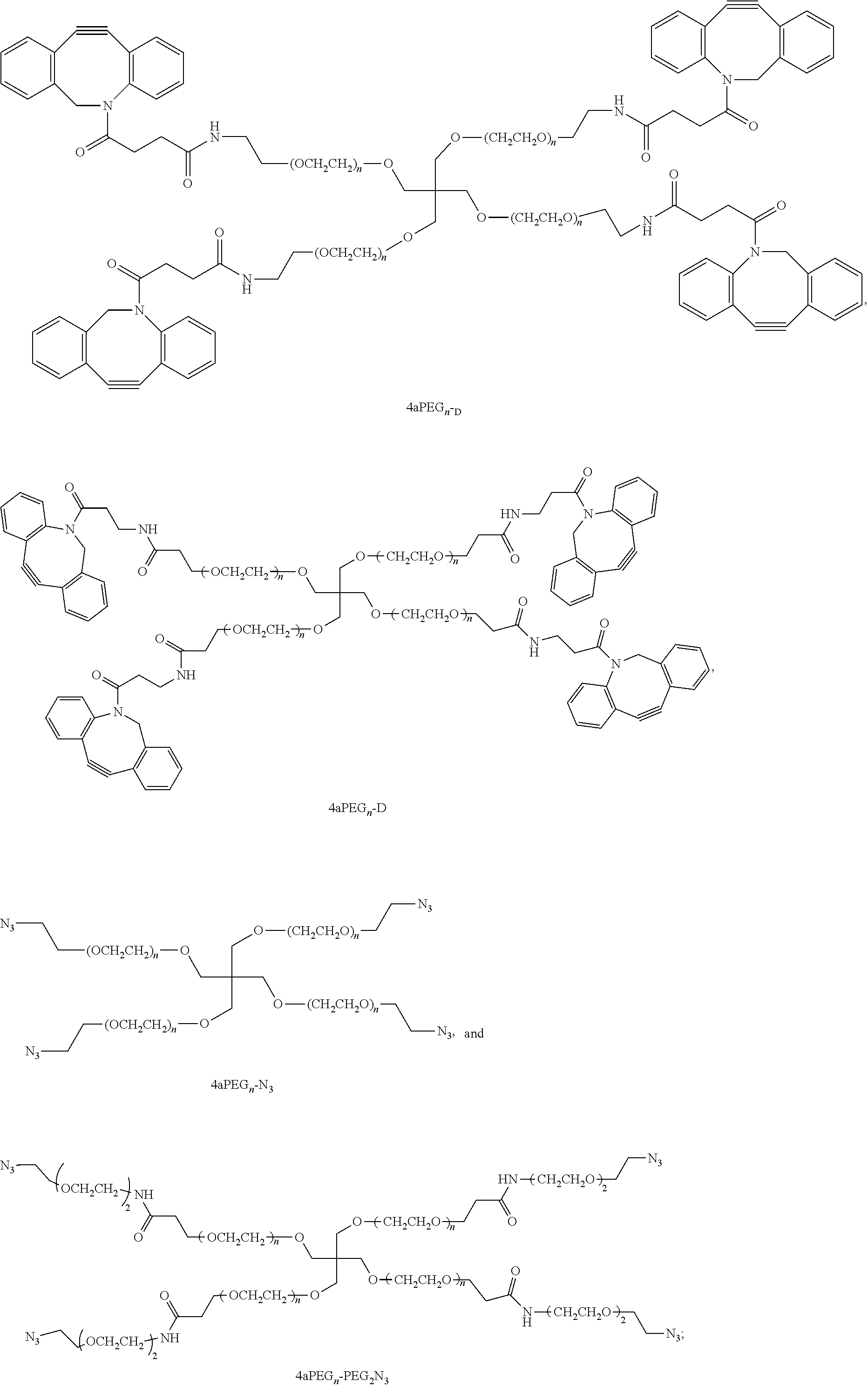

with linker L:

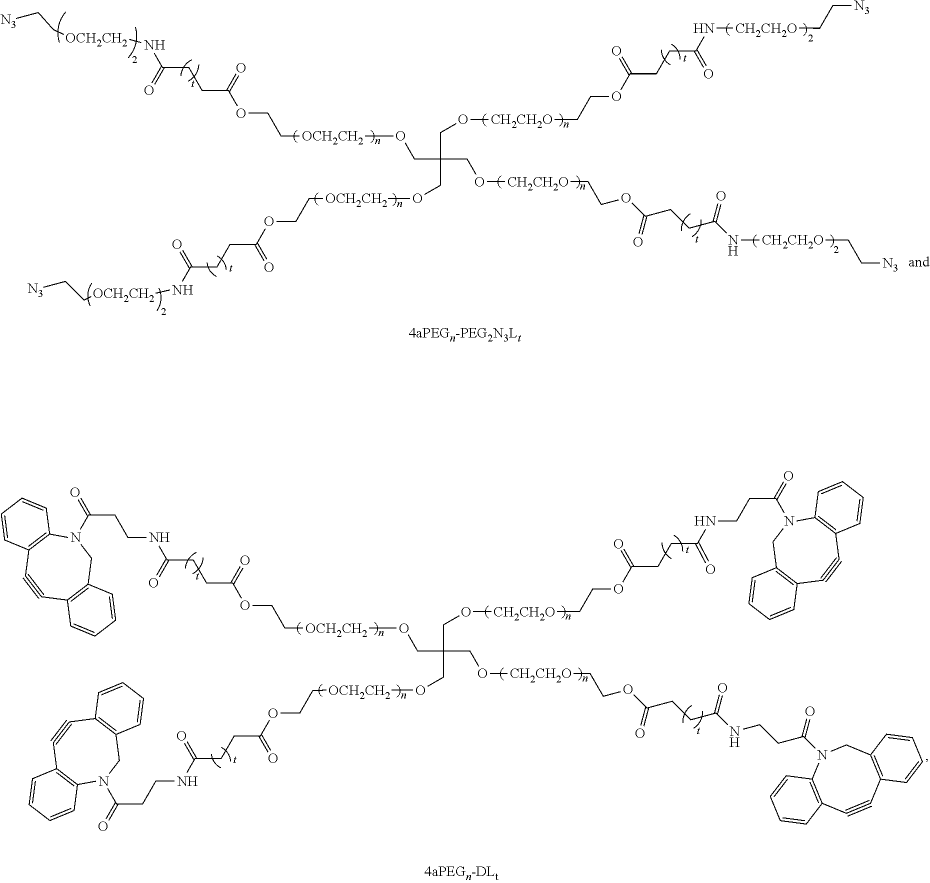

wherein n and t are as defined herein above.

In one embodiment, the first multi-arm precursor having functional groups comprising an azide (N₃) moiety is selected from the group consisting of:

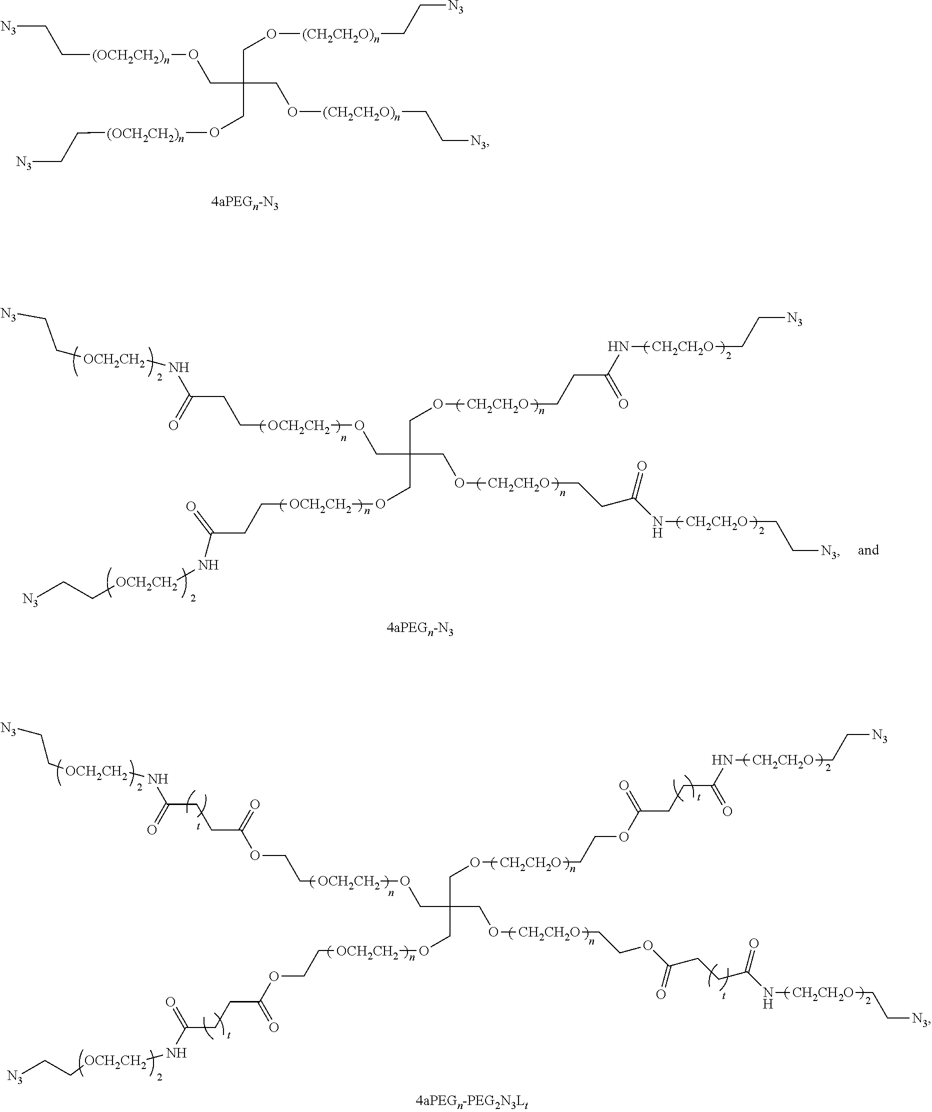

wherein n and t are as defined herein above.

In one embodiment, the second multi-arm precursor having functional groups comprising a dibenzocyclooctyne (DBCO) moiety is selected from the group consisting of:

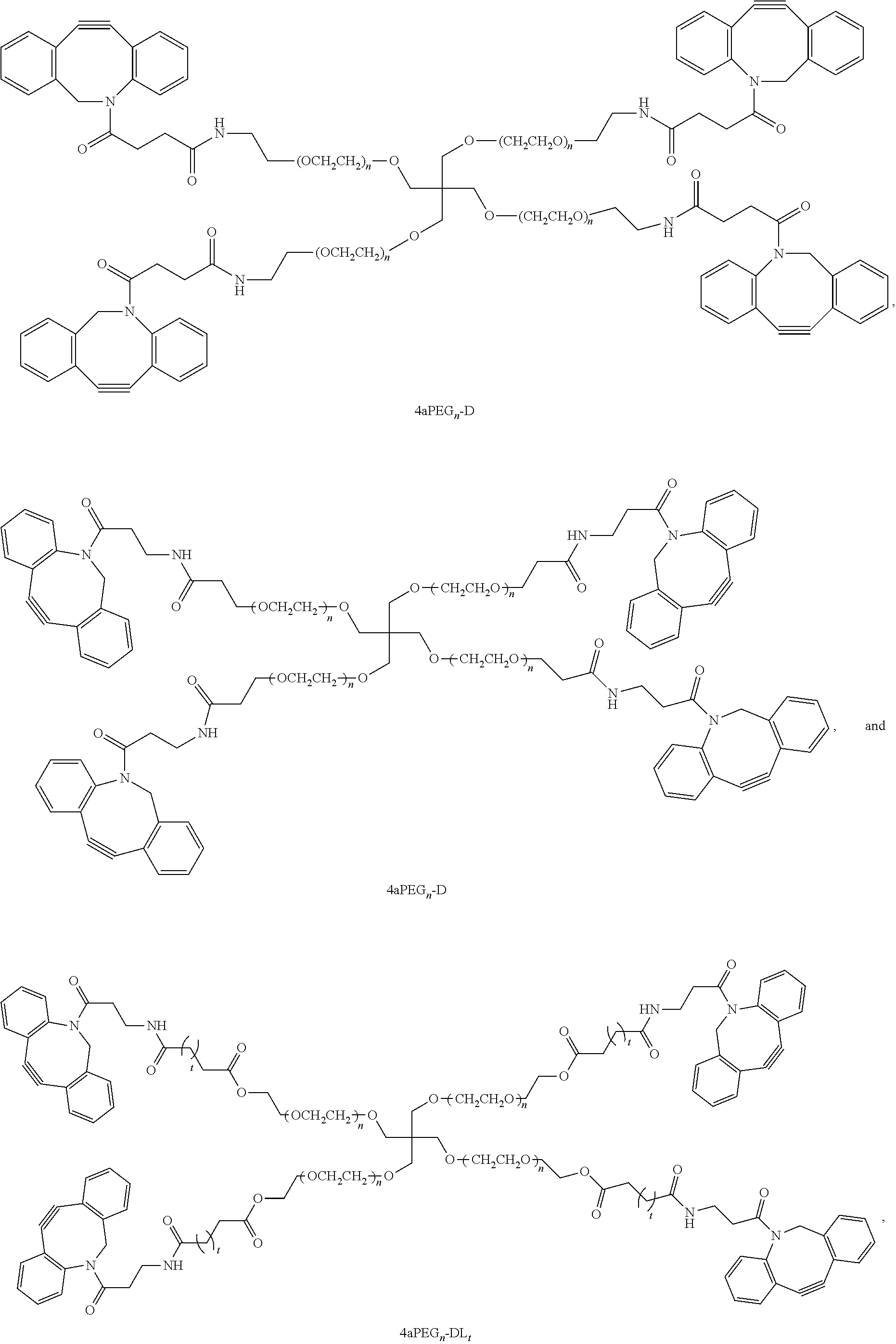

wherein n and t are as defined herein above.

In one embodiment, the polymer network of the bio-orthogonal hydrogel is formed by reacting at least one, preferably one of the following first multi-arm precursors having functional groups comprising an azide (N₃) moiety with at least one, preferably with one of the following second multi-arm precursor having functional groups comprising a dibenzocyclooctyne (DBCO) moiety:

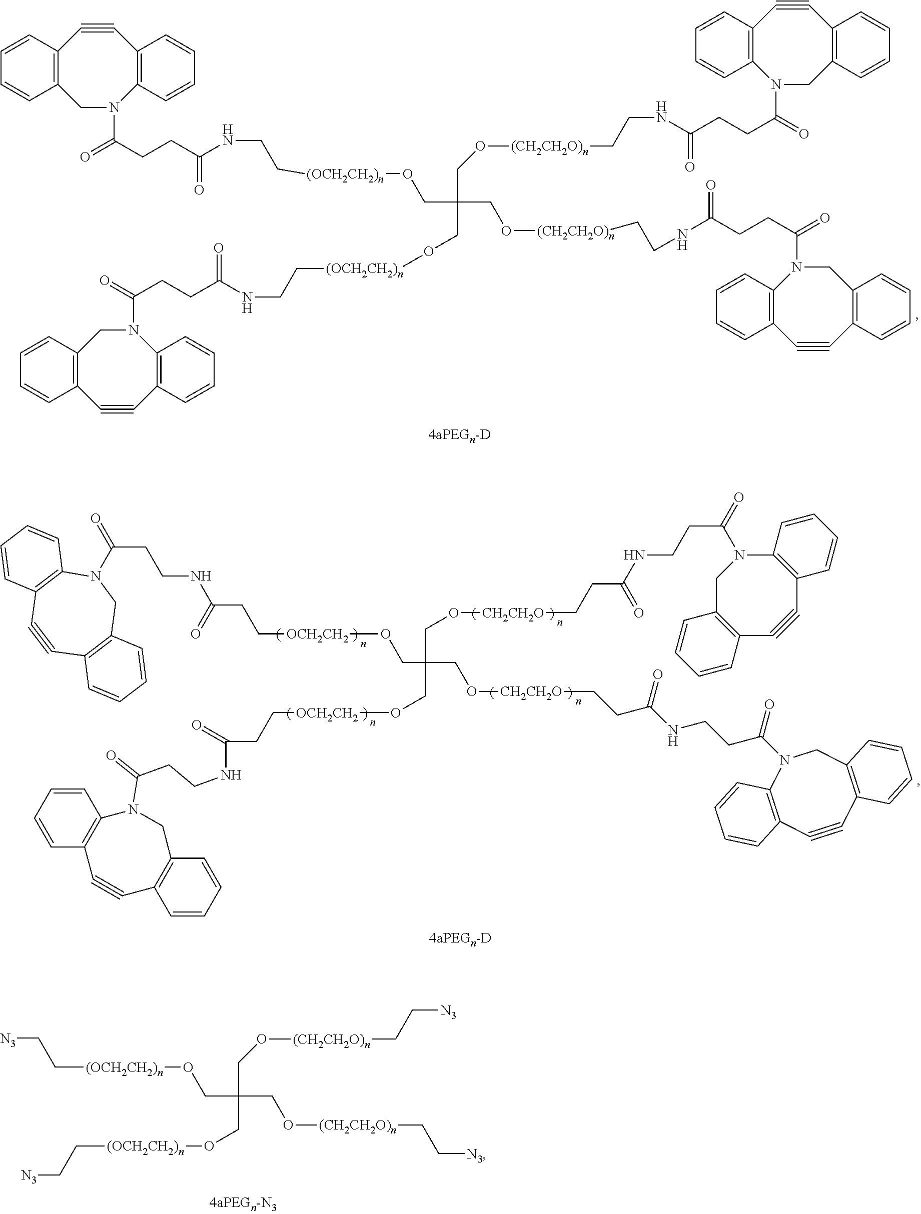

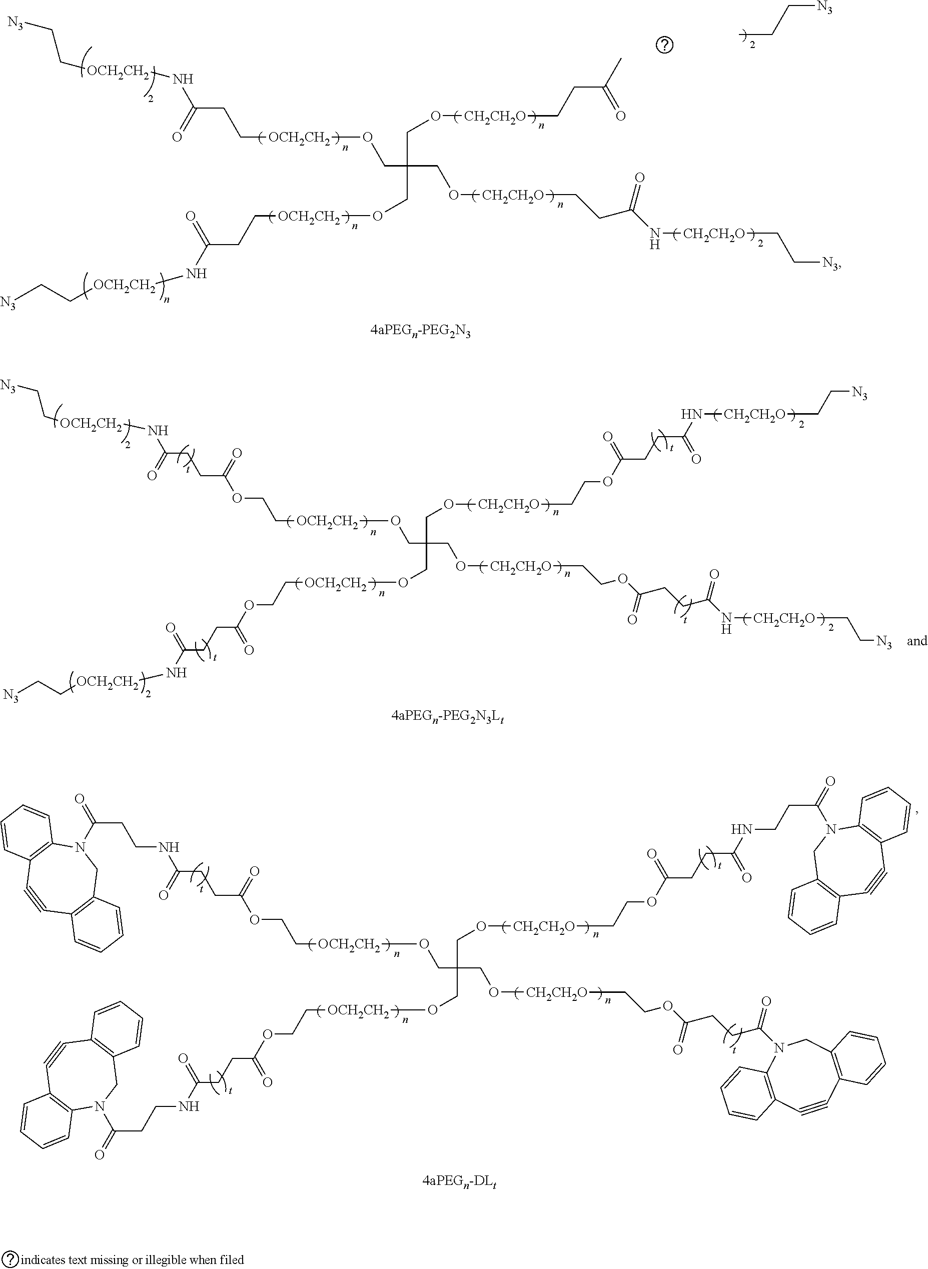

wherein n is 1 to 2300, and t is an integer from 0 to 10.

In one embodiment, the first multi-arm precursor having functional groups comprising an azide (N₃) moiety is selected from the group consisting of 8a10kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ. All compounds are commercially available.

In one embodiment, the second multi-arm precursor having functional groups comprising a dibenzocyclooctyne (DBCO) moiety is selected from the group consisting of 4a20kPEG-D, 6a20kPEG-D, 8a20kPEG-D, 4a20kPEG-DS, 8a10kPEG-DG, 4a20kPEG-DG, 4a20kPEG-DAP, 4a20kPEG-DAZ, 4a20kPEG-DPEG₂₃AZ, and 4a20kPEG-sDG. All compounds are commercially available.

In one embodiment, the polymer network of the bio-orthogonal hydrogel is formed by reacting at least one, preferably one, first multi-arm precursor selected from the group consisting of 8a10kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ with at least one, preferably with one, second multi-arm precursor selected from the group consisting of 4a20kPEG-D, 6a20kPEG-D, 8a20kPEG-D, 4a20kPEG-DS, 8a10kPEG-DG, 4a20kPEG-DG, 4a20kPEG-DAP, 4a20kPEG-DAZ, 4a20kPEG-DPEG₂₃AZ, 4a20kPEG-sDG and 4a20kPEG-PEG₂BG.

In one embodiment, the polymer network of the bio-orthogonal hydrogel is formed by reacting the first multi-arm precursor 4a20kPEG-N₃ with at least one, preferably with one, second multi-arm precursor selected from the group consisting of 4a20kPEG-DG, 4a20kPEG-sDG, 4a20kPEG-PEG₂BG, 4a20kPEG-DAZ, and 4a20kPEG-PEG₂₃DAZ.

In one embodiment, the polymer network of the bio-orthogonal hydrogel is formed by reacting at least one, preferably one of the following first multi-arm precursors having functional groups comprising an azide (N₃) moiety with at least one, preferably with one of the following second multi-arm precursor having functional groups comprising a dibenzocyclooctyne (DBCO) moiety:

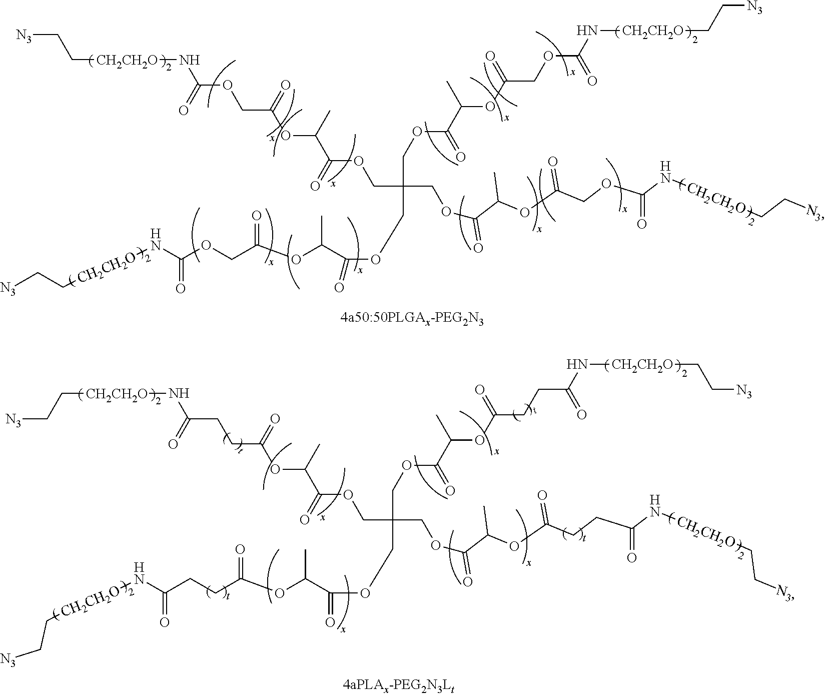

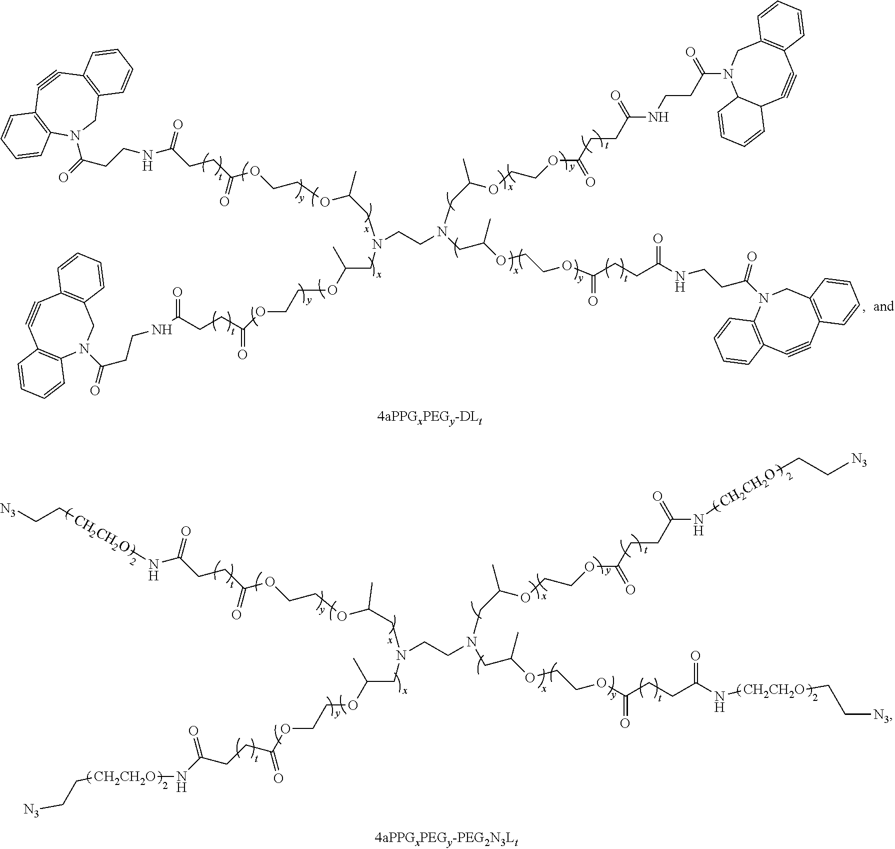

wherein x, y defining the number of PPG, PEG, PLA or PGA units and t are as defined herein.

In one embodiment, the first multi-arm precursor having functional groups comprising an azide (N₃) moiety is selected from the group consisting of 4a18Tetronic1307-PEG₂N₃, 4a20kPLA-PEG₂N₃, 8a20k50:50PLGA-PEG₂N₃, and 4a20kPLA-PEG₂N₃S. All compounds are commercially available.

In one embodiment, the second multi-arm precursor having functional groups comprising a dibenzocyclooctyne (DBCO) moiety is selected from the group consisting of 4a20kPEG-DAZ, 8a20kPEG-D and 4a18kTetronic1307-DAZ. All compounds are commercially available.

In one embodiment, the polymer network of the bio-orthogonal hydrogel is formed by reacting at least one, preferably one, first multi-arm precursors having functional groups comprising an azide (N₃) moiety selected from the group consisting of 4a18Tetronic1307-PEG₂N₃, 4a20kPLA-PEG₂N₃, 8a20k50:50PLGA-PEG₂N₃, and 4a20kPLA-PEG₂N₃S, with at least one, preferably with one, second multi-arm precursor having functional groups comprising a dibenzocyclooctyne (DBCO) moiety selected from the group consisting of 4a20kPEG-DAZ, 8a20kPEG-D and 4a18Tetronic1307-DAZ.

In one embodiment, the polymer network of the invention is not formed by reacting the following compounds:

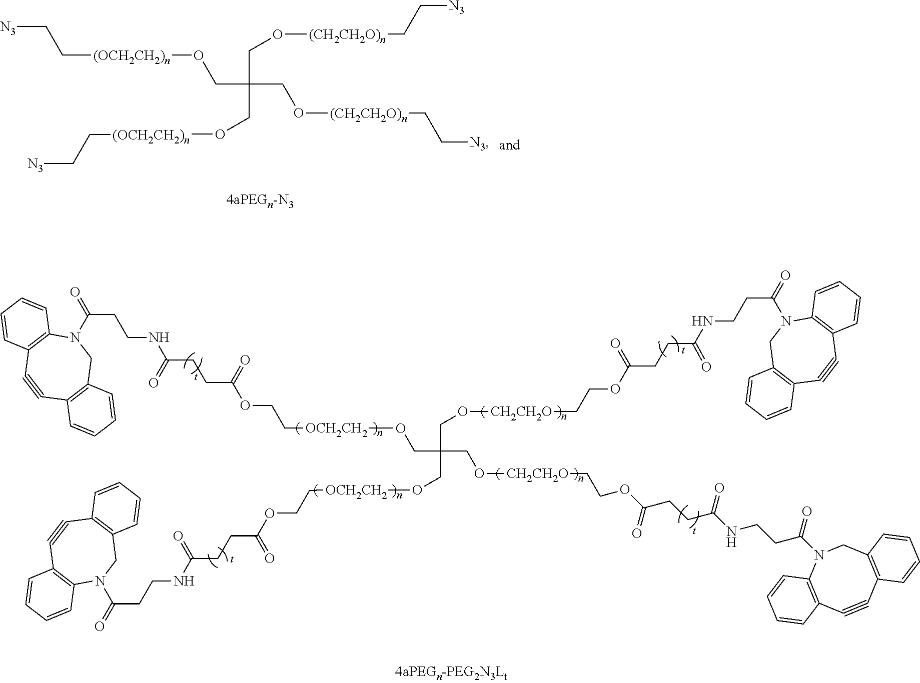

wherein n is 1 to 400, and t is 2.

As described above, in certain embodiments, at least one of the two multi-arm precursors is a polymeric multi-arm precursor. Accordingly, the other multi-arm precursor can be either another polymeric multi-arm precursor, as described above, or a small molecule multi-arm precursor having a molecular weight of less than 1000 Daltons. The small molecule multi-arm precursor can be defined in the same way as the polymer multi-arm precursor, as described above, except that the arms do not comprise polymer units.

After the click chemistry reaction between the first and second functional groups, a covalent connection is formed. This covalent connection may, in certain embodiments, comprise a chemical group selected from triazole and dihydropyridazine. A triazole moiety is obtained when a functional group comprising an azide moiety reacts with a functional group comprising an alkyne moiety. A dihydropyridazine moiety is obtained when a functional group comprising a tetrazine moiety reacts with a functional group comprising a terminal or strained alkene moiety.

Multi-arm precursors having nucleophilic or electrophilic functional groups at the termini of the arms are known in the art. In order to convert these nucleophilic or electrophilic functional groups into functional groups suitable for forming click chemistry connections, click chemistry linkers can be used. These click chemistry linkers contain the functional group suitable for forming click chemistry connections, such as for example an azide or a cyclooctyne, and a nucleophilic or electrophilic functional group such as, for example, an amine (nucleophilic) or a succinimidyl ester (electrophilic). In the conversion, the nucleophilic or electrophilic functional group at the termini of the arms of a multi-arm precursor reacts with the corresponding electrophilic or nucleophilic functional group of the click chemistry linker.

For example, this conversion can be performed by converting polymer arms with NHSS (Succinimidyl Succinate), NHSG (Succinimidyl Glutarate), NHSAP (Succinimidyl Adipate), or NHSAZ (Succinimidyl Azelate) terminal groups into DS (Dibenzocyclooctyne Amido Succinate), DG (Dibenzocyclooctyne Amido Glutarate), DAP (Dibenzocyclooctyne Amido Adipate), or DAZ (Dibenzocyclooctyne Amido Azelate) groups by reacting the NHS group with a DBCO-Amine click chemistry linker such as DBCO-NH₂ having the following structure:

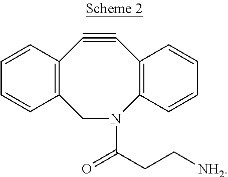 [SMILES: NCCC(=O)N1Cc2ccccc2C#Cc2ccccc21]

Other click chemistry linkers for converting NHS terminated polymer precursors into functionalized precursors suitable for click chemistry are, for example, the more hydrophilic sulfo-DBCO-Amine (sD-NH₂) having the following structure:

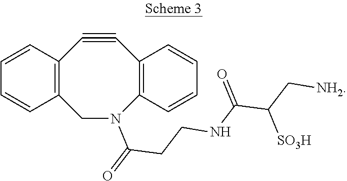 [SMILES: NCC(C(=O)NCCC(=O)N1Cc2ccccc2C#Cc2ccccc21)S(=O)(=O)O]

or PEG extended DBCO-Amines such as DBCO-PEG₂₃-Amine, or bicyclo[6.1.0]nonyne (BCN) amines such as BCN-PEG₂-NH₂ having the following structure:

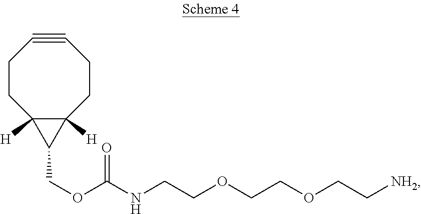 [SMILES: NCCOCCOCCNC(=O)OC[C@@H]1[C@@H]2CCC#CCC[C@@H]21]

and similar commercially available linkers having varying lengths of ethoxylation.

Similarly, a conversion of PEG-NHS termini into a PEG arm terminated with azide groups can be done by reacting the NHS group with an azido-amine click chemistry linker such as azido-PEG₂-NH₂ or the like. Such azido-amine click chemistry linker are commercially available from several vendors and have a structure as shown below:

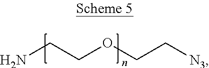

with n defining the number of ethylene glycol repeating units.

The following is a schematic reaction between an exemplary first multi-arm precursor and an exemplary second multi-arm precursor, thereby forming a hydrogel:

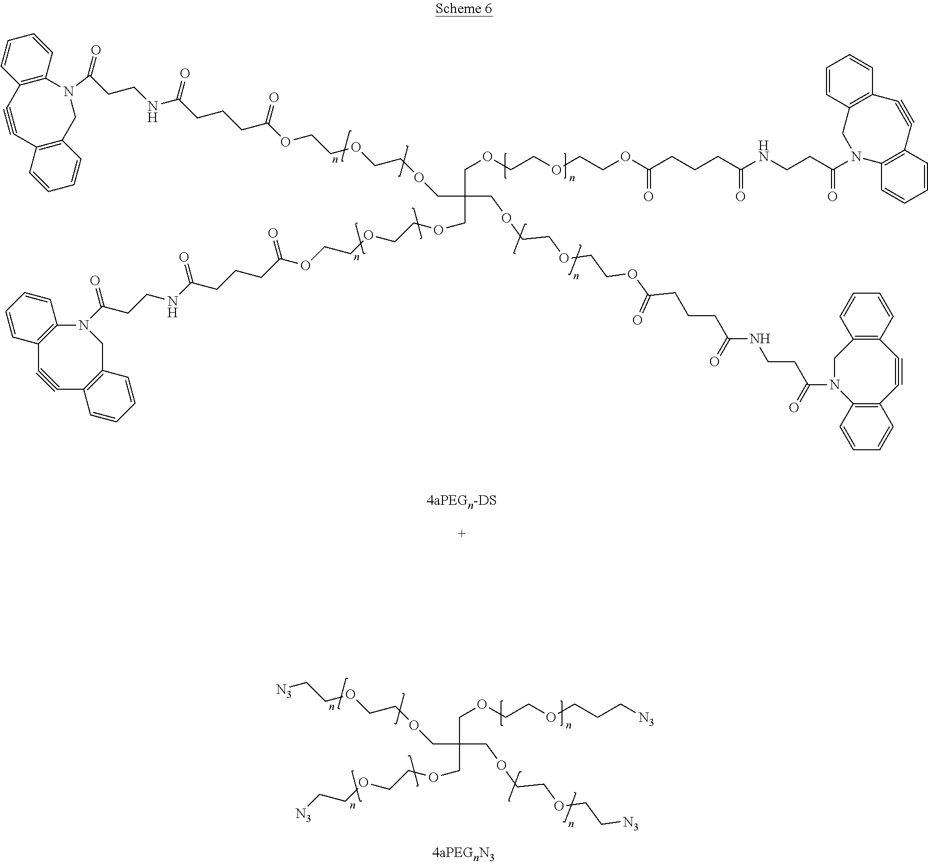

wherein n is as defined herein before.

Since the hydrogel contains hydrolysable ester bonds, it is biodegraded in aqueous environments. The following is an exemplary, schematic hydrolysis reaction of a hydrogel into its degradants.

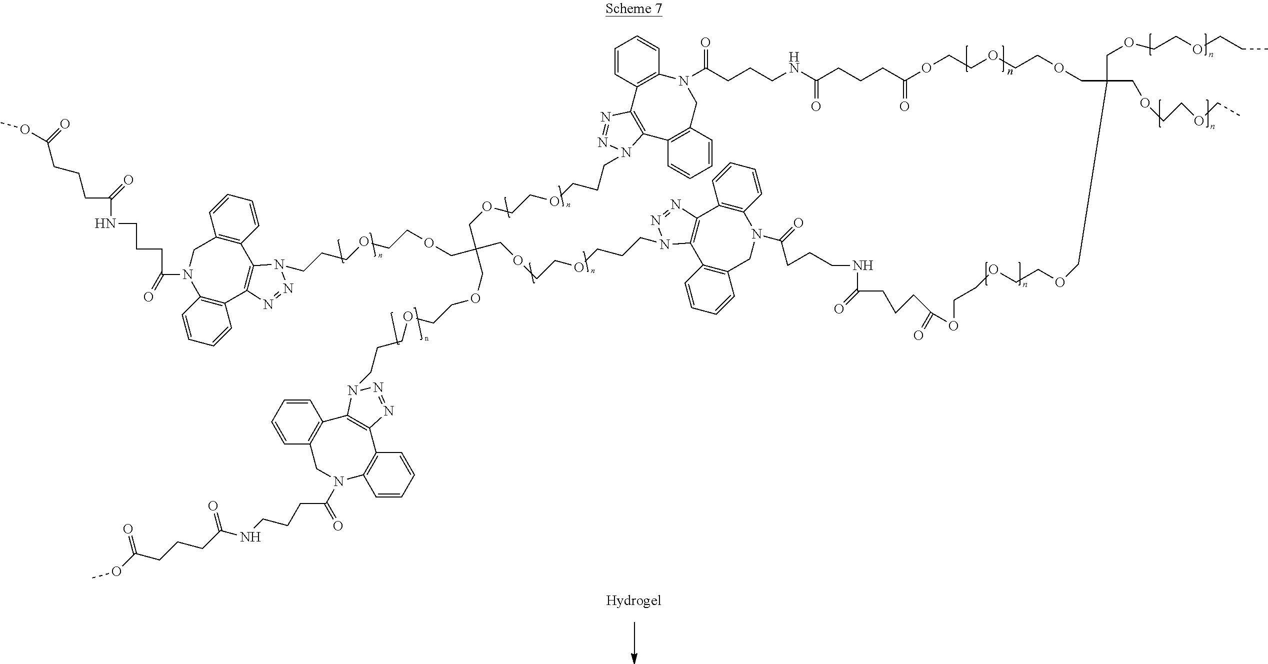

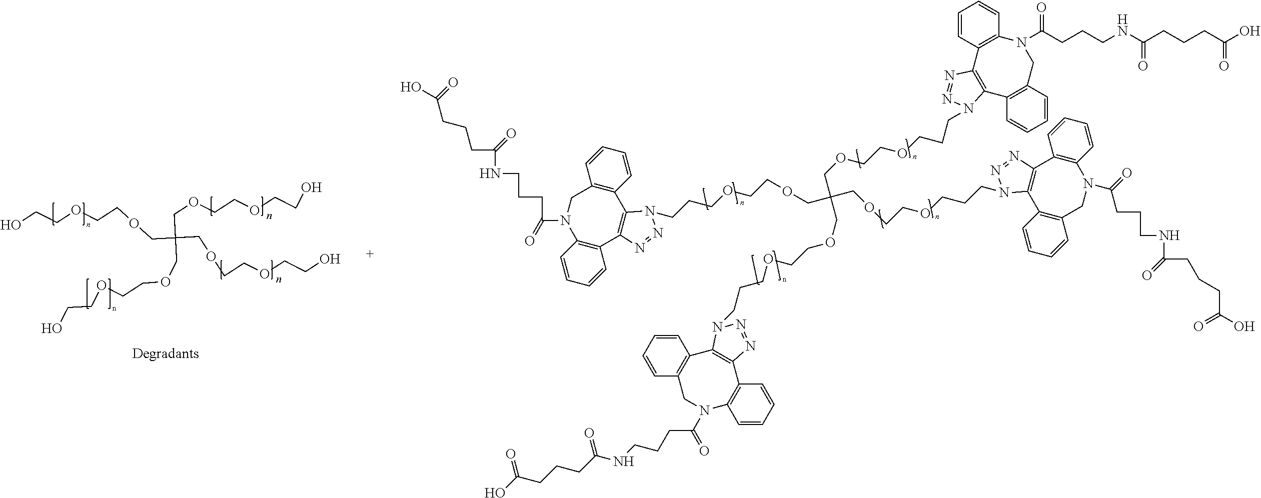

wherein n is as defined herein before.

### Active Agent

The active agent according to the invention can be a therapeutically active agent or a diagnostically active agent, or combinations thereof. It may be a single active agent or a plurality of active agents.

Therapeutically active agents may be selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, protein binders such as nanobodies, affibodies, ankyrins, DARPins, etc., or any combinations thereof.

In some embodiments, steroids may be selected from corticosteroids that can comprise hydrocortisone, loteprednol, cortisol, cortisone, prednisolone, methylprednisolone, dexamethasone, betamethasone, triamcinolone, aldosterone, or fludrocortisone.

In some embodiments, NSAIDs can comprise diclofenac (e.g., diclofenac sodium), flurbiprofen (e.g., flurbiprofen sodium), ketorolac (e.g., ketorolac tromethamine), bromfenac, or nepafenac.

In some embodiments, IOP lowering agents and/or glaucoma medications can comprise prostaglandin analogs (e.g., bimatoprost, latanoprost, travoprost, or latanoprostene bunod), Rho kinase inhibitor (e.g., netarsudil), adrenergic agonists (epinephrine or dipivefrin), beta-adrenergic antagonists also known as beta blockers (e.g., timolol, levobunolol, metipranolol, carteolol, or betaxolol), alpha2-adrenergic agonists (e.g., apraclonidine, brimonidine, or brimonidine tartrate), carbonic anhydrase inhibitors (e.g., brinzolamide, dichlorphenamide, methazolamide acetazolamide, acetazolamide, or dorzolamide), pilocarpine, echothiophate, demercarium, physostigmine, and/or isofluorophate.

In some embodiments, anti-infectives can comprise antibiotics such as ciprofloxacin, tobramycin, erythromycin, ofloxacin, gentamicin, fluoroquinolone antibiotics, moxifloxacin, and/or gatifloxacin; antivirals comprising ganciclovir, idoxuridine, vidarabine, and/or trifluridine; and/or antifungals comprising amphotericin B, natamycin, voriconazole, fluconazole, miconazole, clotrimazole, ketoconazole, posaconazole, echinocandin, caspofungin, and/or micafungin.

In some embodiments, antimetabolites can comprise methotrexate, mycophenolate, or azathioprine. In some embodiments, antifibrotic agents can comprise mitomycin C or 5-fluorouracil.

In some embodiments, angiogenesis inhibitors can comprise anti-VEGF agents (e.g., aflibercept, ranibizumab, bevacizumab), PDGF-B inhibitors (e.g., Fovista®), complement antagonists (e.g., eculizumab), tyrosine kinase inhibitors (e.g., sunitinib, axitinib), and/or integrin antagonists (e.g., natalizumab and vedolizumab). In certain embodiments, the active agent can be selected from peptides selected from the group consisting of Compstatin, APL-1, Fc-III-4C, Beovu (Brolucizumab), Zimura (Avacincaptad Pegol), Pegcetacoplan, Abicipar Pegol, Lampalizumab, Fovista, Risuteganib, AXT107, Elamipretide, THR149, ALM201, VGB3, and Largazole.

In some embodiments, nanobodies can be included in the hydrogels. Nanobodies are described, for example, in Yang et al. (2020), Nanobodies: Next Generation of Cancer Diagnostics and Therapeutics, Front. Oncol. 10:1182, which is incorporated herein by reference in its entirety. Nanobodies may be selected from ⁶⁸GaNOTA-Anti-HER2-VHH1, ⁶⁸GaNOTA-Anti-HER2-VHH1, ^{99m}Tc-NM-02, ¹³¹I-SGMIB-Anti-HER2-VHH1, ⁶⁸GaNOTA-Anti-MMR-VHH2, ^{99m}Tc-Anti-PD-L1, L-DOS47+Doxorubicin, L-DOS47+Cisplatin/Vinorelbine, KN035+Trastuzumab/Docetaxel, KN035, KN044, TC-210 T Cells, CD19/CD20 bispecific CAR T cells, BCMA CAR T cells, or TAS266 nanobodies.

In some embodiments, non-immunoglobulin affinity proteins such as affibodies can be included in the hydrogel delivery systems. Affibody molecules are described, for example, in Ståhl et al., Affibody Molecules in Biotechnological and Medical Applications, Trends in Biotechnology 2017, 35 (8) p. 691-712, which is incorporated herein by reference in its entirety.

In some embodiments, binding proteins such as ankyrins and DARPins can be included in the hydrogel. Ankyrins and DARPins are described, for example, in a review by Caputi et al., Current Opinion in Pharmacology 2020, 51:93-101, which is incorporated herein by reference in its entirety. Ankyrins and DARPins may be selected from MP0250, a tri-specific DARPin drug candidate that can bind VEGF-A and hepatocyte growth factor (HGF) as well as one molecule of MP0250 binding two molecules of human serum albumin (HSA); Abicipar pegol (MP0112 or AGN-150998); Brolucizumab, Ranibizumab, or Aflibercept.

In some embodiments, cytoprotective agents can comprise ebselen, sulforaphane, oltipraz, or dimethyl fumarate. In some embodiments, neuroprotective agents can comprise ursodiol, memantine or acetylcysteine. In some embodiments, anaesthetic agents can comprise lidocaine, proparacaine or bupivacaine.

In some embodiments, the active agent can be dexamethasone, ketorolac, diclofenac, vancomycin, moxifloxacin, gatifloxicin, besifloxacin, travoprost, 5-fluorouracil, methotrexate, mitomycin C, prednisolone, bevacizumab (Avastin®), ranibizumab (Lucentis®), sunitinib, pegaptanib (Macugen®), timolol, latanoprost, brimonidine, nepafenac, bromfenac, triamcinolone, difluprednate, fluocinolide, aflibercept, or combinations thereof. In some embodiments, the agent may be dexamethasone, ketorolac, diclofenac, moxifloxacin, travoprost, 5-fluorouracil, or methotrexate.

In alternative embodiments, active agents that can be utilized in the implants and methods of the present invention include but are not limited to immunosuppressants, complement inhibitors (e.g., C5 inhibitors such as eculizumab or avacincaptad pegol), steroids, anti-inflammatoires such as steroidal and non-steroidal anti-inflammatoires (e.g., COX1 or COX 2 inhibitors), antivirals, antibiotics, anti-glaucoma agents, anti-VEGF agents, analgesics, tyrosine kinase inhibitors, integrin inhibitors, IL-6 blockers, reactive aldehyde species (RASP) inhibitors, nitric oxide donating PgAs, antihistamines, mast cell stabilizers, rho kinase inhibitors, plasma kallikrein inhibitors, BCL-2 blockers, semaphorin antagonists, HtRA1 blockers, IGF-1R inhibitors, VEGF combination agents (multi-specific antiangiogenic agents) and combinations thereof.

Immunosuppressants include but are not limited to cyclosporine, mTOR inhibitors (e.g., rapamycin, tacrolimus, temsirolimus, sirolimus, everolimus, KU-0063794, WYE-354, AZD8055, metformin, or Torin-2), cyclophosphamide, atoposide, thiotepa, methotrexate, azathioprine, mercaptopurine, interferons, infliximab, etanercept, mycophenolate mofetil, 15-deoxyspergualin, thalidomide, glatiramer, leflunomide, vincristine, cytarabine, pharmaceutically acceptable salts thereof and combinations thereof.

Non-steroidal anti-inflammatory compounds include inhibitors of the cyclooxygenase (COX) enzyme such as cyclooxygenase-1 (COX-1) and cyclooxygenase-2 (COX-2) isozymes. General classes of non-steroidal anti-inflammatory compounds include salicylates, propionic acid derivatives, acetic acid derivatives, enolic acid derivatives, and anthranilic acid derivatives. Examples of non-steroidal anti-inflammatory compounds include acetylsalicylic acid, diflunisal, salsalate, ibuprofen, dex-ibuprofen, naproxen, fenoprofen, ketoprofen, dex-ketoprofen, flurbiprofen, oxaprozin, loxoprofen, indomethacin, tolmetin, sulindac, etodolac, ketorolac, diclofenac, aceclofenac, nabumetone, piroxicam, tenoxicam, tenoxicam, loroxicam, phenylbutazone, mefenamic acid, meclofenamic acid, flufenamic acid, tolfenamic acid, celecoxib, pharmaceutically acceptable salts thereof and combinations thereof.

Anti-inflammatory agents that may be utilized in the implants and methods of the present invention may include agents that target inflammatory cytokines such as TNFα, IL-1, IL-4, IL-5, or IL-17, or CD20. Such agents may include etanercept, infliximab, adalimumab, daclizumab, rituximab, tocilizumab, certolizumab pegol, golimumab, pharmaceutically acceptable salts thereof and combinations thereof.

Analgesics that may be utilized in the implants and methods of the present invention include acetaminophen, acetaminosalol, aminochlorthenoxazin, acetylsalicylic 2-amino-4-picoline acid, acetylsalicylsalicylic acid, anileridine, benoxaprofen, benzylmorphine, 5-bromosalicylic acetate acid, bucetin, buprenorphine, butorphanol, capsaicin, cinchophen, ciramadol, clometacin, clonixin, codeine, desomorphine, dezocine, dihydrocodeine, dihydromorphine, dimepheptanol, dipyrocetyl, eptazocine, ethoxazene, ethylmorphine, eugenol, floctafenine, fosfosal, glafenine, hydrocodone, hydromorphone, hydroxypethidine, ibufenac, p-lactophenetide, levorphanol, meptazinol, metazocine, metopon, morphine, nalbuphine, nicomorphine, norlevorphanol, normorphine, oxycodone, oxymorphone, pentazocine, phenazocine, phenocoll, phenoperidine, phenylbutazone, phenylsalicylate, phenylramidol, salicin, salicylamide, tiorphan, tramadol, diacerein, actarit, pharmaceutically acceptable salts thereof and combinations thereof.

Antibiotic that may be utilized in the implants and methods of the present invention include aminoglycosides, penicillins, cephalosporins, fluoroquinolones, macrolides, and combinations thereof. Aminoglycosides may include tobramycin, kanamycin A, amikacin, dibekacin, gentamicin, sisomicin, netilmicin, neomycin B, neomycin C, neomycin E, streptomycin, paramomycin, pharmaceutically acceptable salts thereof and combinations thereof. Penicillins may include amoxicillin, ampicillin, bacampicillin, carbenicillin, cloxacillin, dicloxacillin, flucloxacillin, mezlocillin, nafcillin, oxacillin, penicillin G, penicillin V, piperacillin, pivampicillin, pivmecillinam, ticarcillin, pharmaceutically acceptable salts thereof and combinations thereof. Cephalosporins may include cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime, ceftioxide, pharmaceutically acceptable salts thereof and combinations thereof. Fluoroquinolones may include ciprofloxacin, levofloxacin, gatifloxacin, moxifloxacin, ofloxacin, norfloxacin, pharmaceutically acceptable salts thereof and combinations thereof. Macrolides may include azithromycin, erythromycin, clarithromycin, dirithromycin, oxithromycin, telithromycin, pharmaceutically acceptable salts thereof and combinations thereof.

Antivirals that may be utilized in the implants and methods of the present invention include nucleoside reverse transcriptase inhibitors, non-nucleoside reverse transcriptase inhibitors, fusion inhibitors, integrase inhibitors, nucleoside analogs, protease inhibitors, and reverse transcriptase inhibitors. Examples of antiviral agents include, but are not limited to, abacavir, aciclovir, acyclovir, adefovir, amantadine, amprenavir, ampligen, arbidol, atazanavir, boceprevir, cidofovir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, interferon, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramiding saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, pharmaceutically acceptable salts thereof and combinations thereof.

Steroidal anti-inflammatory agents that may be utilized in the implants and methods of the present invention include dexamethasone, budensonide, triamcinolone, hydrocortisone, fluocinolone, loteprednol, prednisolone, mometasone, fluticasone, rimexolone, fluorometholone, beclomethasone, flunisolide, pharmaceutically acceptable salts thereof and combinations thereof.

Anti-glaucoma agents that may be utilized in the implants and methods of the present invention include beta-blockers such as atenolol propranolol, metipranolol, betaxolol, carteolol, levobetaxolol, levobunolol timolol, pharmaceutically acceptable salts thereof and combinations thereof; adrenergic agonists or sympathomimetic agents such as epinephrine, dipivefrin, clonidine, aparclonidine, brimonidine, pharmaceutically acceptable salts thereof and combinations thereof; parasympathomimetics or cholinergic agonists such as pilocarpine, carbachol, phospholine iodine, physostigmine, pharmaceutically acceptable salts thereof and combinations thereof; carbonic anhydrase inhibitor agents, including topical or systemic agents such as acetozolamide, brinzolamide, dorzolamide; methazolamide, ethoxzolamide, dichlorphenamide, pharmaceutically acceptable salts thereof and combinations thereof; mydriatic-cycloplegic agents such as atropine, cyclopentolate, succinylcholine, homatropine, phenylephrine, scopolamine, tropicamide, pharmaceutically acceptable salts thereof and combinations thereof; prostaglandins such as prostaglandin F2 alpha, antiprostaglandins, prostaglandin precursors, or prostaglandin analog agents such as bimatoprost, latanoprost, travoprost, unoprostone, tafluprost, pharmaceutically acceptable salts thereof and combinations thereof.

Anti-VEGF agents that may be utilized in the implants and methods of the present invention include bevacizumab, pegaptanib, ranibizumab, brolucizumab, conbercept, aflibercept, pharmaceutically acceptable salts thereof and combinations thereof.

Tyrosine kinase inhibitors that may be utilized in the implants and methods of the present invention include deucravacitinib, axitinib, avapritinib, capmatinib, pegimatinib, ripretinib, selpercatinib, selumetinib, tucatinib, entrectinib, erdaftinib, fedratinib, pexidartinib, upadacatinib, zanubrutinib, baricitinib, binimetinib, dacomitinib, fostamatinib, gilteritinib, larotrectinib, lorlatinib, acalabrutinib, brigatinib, midostaurin, neratinib, alectinib, cobimetinib, lenvatinib, osimertinib, ceritinib, nintedanib, afatinib, ibrutinib, trametinib, bosutinib, cabozantinib, ponatinib, regorafenib, tofacitinib, crizotinib, ruxolitinib, vandetanib, pazopanib, lapatinib, nilotinib, dasatinib, sunitinib (vorolanib), sorafenib, erlotinib, gefitinib, imatinib, afatinib, bosutinib, cabozantinib, cediranib, ceritinib, crizotinib, dabrafenib, dasatinib, erlotinib, everolimus, gefitinib, imatinib, lestaurtinib, nilotinib, palbociclib, pazopanib, ponatinib, regorafenib, ruxolitinib, semananib, sirolimus, sorafenib, temsirolimus, tofacitinib, trametinib, vandetanib, and vemurafenib. In another embodiment, the tyrosine kinase inhibitor is a Src family tyrosine kinase inhibitor, such as but not limited to, A419259, AP23451, AP23464, AP23485, AP23588, AZD0424, AZM475271, BMS354825, CGP77675, CU201, ENMD 2076, KB SRC 4, KX2361, KX2-391, MLR 1023, MNS, PCI-32765, PD166285, PD180970, PKC-412, PKI166, PP1, PP2, SRN 004, SU6656, TC-S7003, TG100435, TG100948, TX-1123, VAL 201, WH-4-023, XL 228, altenusin, bosutinib, damnacanthal, dasatinib, herbimycin A, indirubin, neratinib, lavendustin A, pelitinib, piceatannol, saracatinib, SrcI1, foretinib, motesanib, tivozanib, LY2457546, MGCD-265, MGCD-510, tivantinib, AMG458, JNJ-3887, EMD1214063, BMS794833, PHI1665752, SGX-523, INCB280, pharmaceutically acceptable salts thereof and combinations thereof.

Complement pathway modulators that may be utilized in the implants and methods of the present invention include those that target, e.g., C1/C1Q, C3, C3 Convertase, C5, C5 convertase, C5a, C5aR, C6, C7, C8, C9, CD59, Factor B, Factor D, Factor H, Factor P, or a combination thereof. Particular agents may include cinryze, berinert, ruconest, sutimlimab, pegcetacoplan (GA), eculiziumab, ravuilizumab, avacopan, pozelimab, nomacopan, zilucopan, vilobelimab, crovalimab, avacincapted pegol), cemdisiran, BDB-001, tesidolumab, avdoralimab, MOR210, ALXN1720, danicopan, vemircopan, ACH-5228, ACH-5548, BCX-9330, AMY-101, ANX005, ANX007, narsoplimab, iptacopan, CLG561, GT103, ARGX-117, ALXN1820, NGM621, lampalizumab, NGM621, IONIS-FB-Lrx, GEM103, CLG561, pharmaceutically acceptable salts thereof and combinations thereof.

Integrin inhibitors that may be utilized in the implants and methods of the present invention include lifitegrast, vedolizumab, natalizumab, efalizumab, tirofiban, eptifibatide, abciximab, IDL-2965, PLN-74809, PLN-1474, PN-943, 7HP349, MORF-057, OS2966, OTT166, AXT-107, JSM-6427, Risuteganib, THR-687 (D/ced), pharmaceutically acceptable salts thereof and combinations thereof.

Antihistamines that may be utilized in the implants and methods of the present invention include loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, pharmaceutically acceptable salts thereof and combinations thereof.

IL-6 inhibitors that may be utilized in the implants and methods of the present invention include sarilumab, tocilizumab, RG6179, pharmaceutically acceptable salts thereof and combinations thereof.

HtrA1 inhibitors that may be utilized in the implants and methods of the present invention include IC-500, FHTR2163, RG6147, pharmaceutically acceptable salts thereof and combinations thereof.

RASP inhibitors that may be utilized in the implants and methods of the present invention include reproxalap and pharmaceutically acceptable salts thereof.

Rho kinase inhibitors that may be utilized in the implants and methods of the present invention include netardusil, ripasudil, HA-1077, Y-27632, H-1152P, INS-115644, Y-39983, SB772077BS, LX71D1, AR-12286, AMA-0076, AR-13533, pharmaceutically acceptable salts thereof and combinations thereof.

Plasma kallikrein inhibitors that may be utilized in the implants and methods of the present invention include ecallantide, lanadelumab, berotralstat, ATN-249, KVD900, KVD824, THR-149, pharmaceutically acceptable salts thereof and combinations thereof.

Nitric Oxide Donating PgAs that may be utilized in the implants and methods of the present invention include Latanoprostene Bunod, NCX470, NCX125, pharmaceutically acceptable salts thereof and combinations thereof.

Mast Cell Stabilizers that may be utilized in the implants and methods of the present invention include lodoxamide, nedocromil, pemirolast, cromolyn (e.g., chromolyn sodium), pharmaceutically acceptable salts thereof and combinations thereof.

IGF-1R Inhibitors that may be utilized in the implants and methods of the present invention include teprotutumab, VRDN-001, VRDN-002, VRDN-003, ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, B1I885578, B1893923, TT-100, XL-228, A-928605, pharmaceutically acceptable salts thereof and combinations thereof.

TRPV1 antagonists that may be utilized in the implants and methods of the present invention include asivatrep, V116517, fused azabicyclic, heterocyclic, and amide compounds as described, for example, in U.S. Patent Application No. 2004/0157849, U.S. Patent Application No. 2004/0209884, U.S. Patent Application No. 2005/0113576, International Patent Application No. WO 05/016890, U.S. Patent Application No. 2004/0254188, U.S. Patent Application No. 2005/0043351, International Patent Application No. WO 05/040121, U.S. Patent Application No. 2005/0085512, and Gomtsyan et al., 2005, J. Med. Chem. 48:744-752; fused pyridine derivatives as described, for example, in U.S. Patent Application No. 2004/0138454; pyridyl piperazinyl ureas as described, for example, in Swanson et al., 2005, J. Med. Chem. 48:1857-1872 and U.S. Patent Application No. 2005/0049241, as well as AMG8163 (Bannon et al., 2005, 11.sup.th World Congress on Pain) and BCTC (Sun et al., 2003, Chem. Lett. 13:3611-3616); 2-(piperazine-1-yl)-1H-Benzimidazole; pyridazinylpiperazines; urea derivatives as describe, for example, in U.S. Patent Application No. 2005/0107388, U.S. Patent Application No. 2005/0187291, and U.S. Patent Application No. 2005/0154230, as well as A-425619 (El Kouhen et al., 2005, J. Pharmacol. Exp. Ther. 314:400-409); cinnamides, including SB-366791 (Gunthorpe et al., 2004, Neuropharmacology 46:133-149) and AMG 9810 (Gawa et al., 2005, J. Pharmacol. Exp. Ther. 313:474-484).

In some embodiments, TRPV1 antagonists useful in the methods and compositions as disclosed herein include, for example, TRPV-1 antagonists include capsazepine, (E)-3-(4-t-butylphenyl)-N-(2,3-dihydrobenzo[b][1,4]dioxin-6-yl) acrylamide (commercially available for example as AMG9810 from Tocris Bioscience, Bristol, United Kingdom), and 4-tertiary butyl cyclohexane (commercially available as SYMSITIVE 1609 from Symrise GmbH of Holzminden, Germany, as well as TRPV1 antagonists as disclosed in U.S. Pat. Nos. 8,815,930, 6,933,311, 7,767,705 and U.S. Pat. App. Pub. Nos. 2010/0249203 and 2011/0104301, International Application WO/2008/013861.

In some embodiments, TRPV1 antagonists useful in the methods and compositions and devices as disclosed herein include AMG-517 and AMG-628 (Amgen Inc., Thousand Oaks, Calif.). TRPV1 antagonists useful in the present application are also described, for example, in International Patent Application No. WO 2006065484; International Patent Application No. WO 2003070247; U.S. Patent Application No. US2005080095; and International Patent Application No. WO 2005007642. Additional TRPV1 antagonists useful in the methods and compositions and devices as disclosed herein include TRPV1 antagonists: ABT-102, AMG8562, AMG9810, BCTC, SB366791, JNJ17203212, I-TTX, JYL-1421, A-425619, N-[4-[6-[4 (Trifluoromethyl)phenyl)pyrimidin-4-yloxy]benzothiazol-2-yl]acetamide (also known as AL-49975 or AMG-517), (R)—N-(4-(6-(4-(1-(4-fluorophenyl)ethyl) piperazin-1-yl)pyrimidin-4-yloxy)benzo[d]thiazol-2-yl) acetamide (AL-49976, also known as AMG-628), pharmaceutically acceptable salts thereof and combinations thereof.

Other TRPV1 antagonists useful in the methods and compositions and devices as disclosed herein are those that have a low inhibitory activity against CYP3A4, such as, e.g., 1-(2-(3,3-dimethylbutyl)-4-(trifluoromethyl)benzyl)-3-(1-methyl-1H-in-dazol-4-yl)urea; methyl 2,2-dimethyl-4-(2-((3-(1-methyl-1H-indazol-4-yl) ureido)methyl)-5-(trifluo-romethyl)phenyl) butanoate; 1-(2-(4-hydroxy-3,3-dimethylbutyl)-4-(trifluoromethyl)benzyl)-3-(1-methyl-1H-indazol-4-yl)urea; 2,2-dimethyl-4-(2-((3-(1-methyl-1H-indazol-4-yl) ureido)methyl)-5-trifluor-omethyl)phenyl) butanoic acid; 1-[4-Chloro-3-(3,3-dimethylbutyl)benzyl]-3-(1-methyl-1H-indazol-4-yl)urea-; 1-(2-isobutyl-4-(trifluoromethyl)benzyl)-3-(1-methyl-1H-indazol-4-yl)urea; 1-(2-isopropyl-4-(trifluoromethyl)benzyl)-3-(1-methyl-1H-indazol-4-yl)urea; 1-(4-Chloro-3-isopropylbenzyl)-3-(1-methyl-1H-indazol-4-yl)urea, pharmaceutically acceptable salts thereof and combinations thereof.

TrkA antagonists that may be utilized in the implants and methods of the present invention include VM902A, Larotrectinib, Entrectinib, Selitrectinib (LOXO-195, BAY 2731954), repotrectinib (TPX-0005), pharmaceutically acceptable salts thereof and combinations thereof.

For the purposes of the present invention, an active agent includes all its possible forms, including free acid, free base, polymorphs, pharmaceutically acceptable salts, anhydrites, hydrates, other solvates, stereoisomers, crystalline forms, cocrystals, pro-drugs, conjugates (e.g., pegylated compounds), complexes and mixtures thereof.

Diagnostically active agents may be, e.g., imaging agents, markers, or visualization agents. Generally, diagnostic agents may be substances used to examine the body in order to detect impairment of its normal functions. In some cases, diagnostic agents may be agents with a functional purpose, such as for use in the detection of ocular deformities, ailments, and pathophysiological aspects. For example, the diagnostic agent may be an important and effective diagnostic adjuvant, such as a dye (e.g., fluorescein dye, indocyanine green, trypan blue, a dark quencher such as a cyanine dye, an azo dye, an acridine, a fluorene, an oxazine, a phenanthridine, a naphthalimide, a rhodamine, a benzopyrone, a perylene, a benzanthrone, pra benzoxanthrone), to aid in visualization of ocular tissues. The diagnostic agent may comprise paramagnetic molecules, fluorescent compounds, magnetic molecules, radionuclides, x-ray imaging agents, and/or contrast media. In some embodiments, a diagnostic agent may include radiopharmaceuticals, contrast agents for use in imaging techniques, allergen extracts, activated charcoal, different testing strips (e.g., cholesterol, ethanol, and glucose), pregnancy test, breath test with urea 13C, and various stains/markers. In some embodiments, the labelling moiety is a fluorescent dye or a dark quencher, selected from the group consisting of a coumarin, a cyanine dye, an azo dye, an acridine, a fluorene, an oxazine, a phenanthridine, a naphthalimide, a rhodamine, a benzopyrone, a perylene, a benzanthrone, and a benzoxanthrone. In particular non-limiting embodiments, the fluorescent dye is or is the residue of a compound selected from the group consisting of Coumarin, Fluorescein, Cyanine 3 (Cy3), Cyanine 5 (Cy5), Cyanine 7 (Cy7), Alexa dyes, bodipy derivatives, (E)-2-(4-(phenyldiazenyl) phenoxy) acetic acid, 3-(3′,3′-dimethyl-6-nitrospiro[chromene-2,2′-indolin]-1′-yl) propanoate (Spiropyran), 3,5-dihydroxybenzoate and (E)-2-(4-(phenyldiazenyl) phenoxy) acetic acid, or combinations thereof.

In certain embodiments of the invention, the active agent may be dispersed, embedded or encapsulated in the hydrogel. In certain embodiments thereof, the active agent may be in particulate form. In certain embodiments, the active agent may be conjugated by a covalent bond to click chemistry functional groups in the polymer network, such as additional functional groups that are unreacted or additionally provided on the arms of polymer units.

In embodiments where the active agent is used in particulate form, the active agent particles may be micronized particles, e.g., having a D50 particle size of less than about 15 μm, or less than 10 μm and/or a D99 particle size of less than about 100 μm, or less than about 50 μm, or a D90 particle size of about 50 μm or less, or 5 μm or less and/or a D98 particle size of about 10 μm or less. In other embodiments the active agent particles may be nanosized particles, e.g., having a D50 particle size of less than about 100 nm, or less than about 50 nm, and/or a D99 particle size of less than about 50 nm, or a D90 particle size of about 5 nm or less and/or a D98 particle size of about 10 nm or less. Particle sizes are determined as disclosed in the “Definitions” section herein.

According to the present invention, the drug-delivery system comprising the hydrogel may be designed as desired for the intended use and therapeutic application. In certain embodiments, the hydrogel comprises from 1 to 50 wt.-% of the active agent, or 5-50 wt.-%, 5-40 wt.-%, 10-30 wt.-%, or 10-25 wt.-%; wherein the weight percentages are based on the total dry weight of the hydrogel or drug-delivery system, respectively.

In certain embodiments, the hydrogel drug-delivery system according to the present invention in a dried state contains from about 15% to about 80% by weight of the active agent based on the total weight of the hydrogel and from about 20% to about 60% by weight polymer units based on the total weight of the hydrogel, or from about 30% to about 65% by weight of the active agent based on the total weight of the insert and from about 25% to about 50% by weight polymer units based on the total weight of the insert, or from about 45% to about 55% by weight of the active agent based on the total weight of the hydrogel and from about 37% to about 47% by weight polymer units based on the total weight of the hydrogel.

In one further particular embodiment, on a dry weight basis the active agent to PEG or polymer ratio is from about 50% to 60% by weight active agent to approximately 40% by weight PEG or polymer based on the total weight of the insert, the balance being phosphate salt and other excipients.

In one embodiment of the invention, the hydrogel after being formed and prior to being dried, i.e., in a wet state, comprises from about 3% by weight to about 20% by weight polyethylene glycol representing the polyethylene glycol weight divided by the fluid weight×100. In one embodiment, the hydrogel in a wet state comprises about 7.5% by weight to about 15% by weight polyethylene glycol representing the polyethylene glycol weight divided by the fluid weight×100.

In certain embodiments, the hydrogel is dried after its manufacture, and the water content of the hydrogel in its dry (dehydrated/dried) state, may be very low, such as not more than 1% by weight of water. In other words, in certain embodiments, the hydrogel in a dried state contains not more than about 1% by weight water. The water content may in certain embodiments also be lower than that, e.g., not more than 0.25% by weight or not more than 0.1% by weight, based on the total weight of the hydrogel.

### Characterization

Characterization and purification of the click chemistry functionalized precursors can be done by ¹H-NMR (1H-Nuclear Magnetic Resonance) and HPLC-UV (High Pressure Liquid Chromatography with Ultraviolet Absorption Detection). In an aspect of the methods of the invention, NHS functionalized magnetic beads may be used to remove residual, unreacted DBCO-Amine from the functionalized precursor products.

Other purification methods for the hydrogel precursors obtained in the synthesis methods as described herein include, for example, dialysis, SEC column filtration, or (U)HPLC.

Dialysis is a common purification method based on separating molecules in solution by the difference in their rates of diffusion through a semipermeable membrane, such as a dialysis tubing. Dialysis tubings are commercially available for example from Spectra/Por® Float-A-Lyzer G2 Dialysis Devices, Spectrum® Laboratories, with several different molecular weight cutoffs as desired for the particular separation task. By selecting proper molecular weight cutoff dialysis tubing and membranes, if necessary, in a series of separation steps with different cut-off dialysis tubings, it is possible to remove most of the impurities, such as excess amount of not reacted precursors from the product.

Another purification method that can be applied in certain aspects of the present invention is the use of size exclusion chromatography (SEC), such as a SEC column. For example, Zeba™ Spin Desalting columns (from Thermo Fisher Scientific) designed for protein purification to remove salts and small size impurities can be used for purification of hydrogel precursor reaction mixtures. Columns with different pore size may be used. The purification mechanism is based on size exclusion chromatography, in which small particles will be trapped in the pore on the immobile phase material, and particles with large size will elute through the column and collected in purified form.

The resulting purified products can be characterized by Ultra-high-performance liquid chromatography ((U)HPLC), which is an efficient technique which offers more sensitive analysis with good chromatographic separation and resolution of analytes. It provides benefits including fast analysis, high-resolution separations, reduced solvent and sample usage, enhanced sensitivity and precision, etc. Based on calibration curves and a comparison of the solutions before and after the purification treatment, the amounts of desired product in the purified solution can be determined by peak area integration.

Another parameter, particularly important for in-situ gel forming drug delivery systems of embodiments of the invention is the gel time, i.e. the time from bringing together and mixing the reacting precursor solutions (and the at least one active agent) for about 10 seconds at room temperature to final formation of the (solid) gel. For in-situ applications, a handling time to ensure uniform mixing, minimizing of bubbles and load the needles on the syringe should be at about 1 to 2 minutes, so that the gel time should be more than this handling time. After handling, the gel time can be controllable in certain embodiments as further described herein to provide an additional gel time (in addition to the mixing and handling time) as follows:
Very Fast (0.5s-1 min) for injections in liquid tissues (such as the vitreous humor). In this way the gel forms a shell very quickly and not get diluted within the liquid environment;
Moderate (1-2 mins) for injections in soft tissue;
Slow (1-5 mins) for injections harder tissues;

Thus, for embodiments involving in-situ applications, the overall gel time of the reaction mixture should be from about 1 to 10 minutes, such as 2 to 9 minutes, or at least about 1.5, 2, 3, 4, 5, 6, 7, or 8 minutes.

### Interpenetrating Networks

The use of the click chemistry concept into the hydrogel synthesis allows for the combination of two types of gel crosslinking chemistries in one pot reactions. For instance, in certain embodiments of the present invention, interpenetrating networks of hydrogels from precursors crosslinked by click chemistry such as the DBCO-Azide chemistry described herein, and hydrogels formed from precursors crosslinked by NHS-NH₂ electrophile-nucleophile chemistry can be created.

Since both crosslinking functional groups do not react with each other, the provision of four precursors with each having another type of functional groups according to these crosslinking reactions will crosslink into two differently crosslinked gels that interpenetrate each other.

The different types of networks can have a different gel time and molecular weight between crosslinks with the NHS-NH₂ inside another PEG network with the click chemistry crosslinks. The NHS-NH₂ chemistry does not interact with the DBCO-Azide creating two distinct hydrogel matrices with different mechanical properties entangled within each other. Entanglement of two types of crosslinked macromolecules in the gel allows for lower molecular weight building blocks at the same number of arms of the precursors used, or use of lower number of arm precursors at the same or higher overall molecular weight of the interpenetrating network gel. In certain embodiments, interpenetrating embodiments have a swelling ratio about equivalent to the average of the two separate gels, at the same polymer concentration. In certain embodiments, interpenetrating networks are used for delivery and release of antibodies.

Furthermore, in embodiments of the invention, interpenetrating networks can be formed from more than two click chemistry reactive group functionalized precursors. For example, a DBCO functionalized multi-arm PEG first precursor may be reacted with two or more different second precursors, each bearing another DBCO-reactive functional group such as azide and tetrazine functional groups. In one embodiment, a DBCO functionalized multi-arm PEG first precursor is reacted with a mixture of a tetrazine functionalized multi-arm PEG second precursor in the presence of an azide functionalized multi-arm PEG third precursor. As shown in Example 10 below, such ternary systems allow for easily controlling the gel time by varying the concentration of the third precursor concentration, such as the azide precursor concentration. Without wishing to be bound by a theory, it is believed that such systems with more than two reacting species form interpenetrating networks of differently clicked precursors in competing reactions. Using a third precursor of can thus be used to slow down or accelerate the gel time of the sustained drug delivery system reaction mixture, so that tailored gel time can be provided for different in-situ gel forming applications.

The use of a third (or more) click chemistry reactive precursor can provide control over solution viscosity and gel time. Fast gel times (such as about 10 seconds to 1 minute) can be suitably used for in-situ gel forming injections into liquid tissues such as the vitreous humor or blood, without the gel getting diluted within the liquid environment. Shorter to moderate gel times (such as about 1 to 2, or 1 to 3 minutes) may be preferred for injections into soft tissue, and slow gel times (such as about 1 to 8, or 2 to 5 minutes) may be more suitably used for in-situ gel forming injections into harder tissue.

### Release Kinetics

In certain embodiments, the hydrogels of the invention can be used for sustained release drug-delivery. The hydrogel drug delivery system may be adapted to modify the release of an active agent dispersed in the hydrogel by several measures. For example, tailoring or suitably selecting the precursor components, such as length and molecular weight of polymeric arms, the number of arms, the types of polymer units such as hydrophobic and hydrophilic polymer units and combinations thereof, the type and length of linkers used, etc., can have an influence on active agent release.

In certain embodiments, the linker of Formula (i) used in hydrogel structure introduces hydrolysable bonds into the hydrogel that can be used to modify the degradation rate of the hydrogel and/or the release rate of active agents from the hydrogel when it degrades in-vivo. For example, the rate of biodegradation/hydrolyzation of ester bonds at these linkers decreases from succinate (C4) to azelate (C9) linkers. The shorter the diacid linker chain length, the faster the ester bonds it forms are hydrolyzed. Thus, the hydrolysis rate decreases from SS>SG>SAP>SAZ>SGA ester bonds. In embodiments of the invention, this can be used to control the degradation rate of the hydrogel and/or the release of active agents dispersed therein. For example, esters formed from succinimidyl succinate (NHSS) groups can degrade in the order of a few days, while esters of succinimidyl glutarate (NHSG) groups degrade in the order of weeks. Different linkers may be used within the hydrogel at different positions to control degradation rates among the crosslinks in the hydrogel, to control the release of the active agent from the hydrogel.

For crosslinks formed by click chemistry reactions, an extension of the spacer structure, e.g., an alkylene chain or a pegylation, between the DBCO/Azide functional group and the functional group with which it binds to a polymeric arm of a precursor can be used in embodiments of the disclosure to delay hydrolyzation of neighboring ester linkages as well. The larger the distance between the DBCO/Azide functional group (or other click chemistry linkage described herein) and the next hydrolysable ester group, the slower the hydrolysis of the ester occurs. Different linkers within the hydrogel precursors can be used to enable degradation control. This can be used to control the half-life of an active agent and to modify the release kinetics.

In certain embodiments, a hydrophobic organic liquid such as an oil may be incorporated into the polymer network of the gel, thus forming an organogel comprising the hydrophobic organic liquid as a continuous phase. The hydrophobic organic liquid can be a biocompatible oil selected from the group consisting of triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyols®), phospholipids, glycerophospholipids, sphingolipids, sterols, prenols, polyketides; hydrophobic biodegradable liquid polymers (such as low molecular weight PLGA, PGA or PLA), low melting point waxes such as plant, animal or synthetic waxes, lanolin, jojoba oil, or combinations thereof.

In certain embodiments, the hydrophobic organic liquid is liquid at human body temperature, such as a temperature of about 37° C. or lower, or liquid in the range of 0° C. to 40° C., or 10° C. to 38° C., or 15° C. to 37° C., or 25° C. to 37° C., or at 37° C., and may have a glass transition temperature and/or a melting temperature equal to or below 45° C., or equal to or below 37° C. The term “liquid” may include viscous fluids having a creamy or wax-like but non-solid appearance. Also, for some hydrophobic organic liquids that undergo hydration in aqueous embodiments such as body fluids the melting point at a certain temperature may be different for the hydrated material than for the non-hydrated. In certain embodiments of the present invention, the hydrated form of such materials are liquid under those conditions as described above.

In certain embodiments, the hydrophobic organic liquid is non-volatile at 37° C. and ambient pressure, non-toxic, and/or biocompatible, and/or capable of being cleared from an implantation site, metabolized and/or cleared unchanged from the body.

The active agent can be dissolved in the hydrophobic organic liquid, or dispersed in the form or particles in the hydrophobic organic liquid. In certain aspects, where the active agent is an oil per se, such as travoprost, the active agent is the hydrophobic organic liquid itself, or forms at least a part thereof. In such embodiments, the oil can be used as a carrier for controlling release of the active agent by diffusion of the oil into the bodily environment.

In certain embodiments, the sustained release drug-delivery hydrogel of the present invention is formulated to make an active agent available over an extended period of time, thereby allowing a reduction in dosing frequency compared to an immediate release dosage form, such as for example a solution of an active agent that is topically applied onto the eye (i.e., eye drops). In certain embodiments, the release of the active agent comprises constant active agent release, tapered active agent release as well as any combination thereof such as a constant active agent release followed by a tapered active agent release. The “sustained release” may be measured in-vitro in an aqueous solution under physiological conditions such as at pH 7.2-7.4 and 37° C. and is considered to be the same or substantially the same when the sustained release drug-delivery hydrogel is administered in-vivo to a subject.

In various embodiments of the present invention, the active agent release follows zero order release kinetics or substantially zero order release kinetics, preferably without a “burst” of active agent at the beginning of the period.

Embodiments of the present invention may provide for a release of a therapeutically effective amount of the active agent for a period of time, such as up to 1 year, up to 9 months, up to 6 months, up to 3 months, up to 1 month, or up to about 25 days after administration. Other embodiments of the present invention may provide for a release of a therapeutically effective amount of the active agent of up to about 14 days, or up to about 21 days after administration, or a release of a therapeutically effective amount of the active agent for a period of about 6 hours or longer after administration, or for a period of about 12 hours, or 24 hours or longer or about 48 or longer, or about 72 hours or longer or about 7 days or longer, or about 10 days or longer after administration. The present invention contemplates all of the above lower and higher time periods in any combination of ranges.

Some aspects of the present disclosure are directed to a pharmaceutically acceptable hydrogel drug delivery system for controlled release of a an active agent dispersed therein, wherein the controlled release is characterized by: the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 2 days.

In one embodiment, the controlled release of the active agent is characterized by the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 2 days. In another embodiment, the amount of the active agent released on day 1 is from 0 to 25%, 0 to 20% 0 to 10%, 0 to 5%, or about 0% of the total amount of the active agent, the amount of the active agent released per day from day 2 until the last day of release is from 0 to 50% of the total amount of the active agent, and/or the number of days required for 100% release of the total amount of the active agent is at least 3 days but no greater than 30 days, 25 days, or no greater than 16 days.

When in contact with aqueous body fluids, the hydrogels of certain embodiments of the present invention swell by taking up water, particularly in case of dried hydrogels/xerogels. The degree of swelling is largely determined by the gel-forming components used hydrophilicity. Swelling may result in an increase of length and/or diameter dimensions of shaped hydrogels according to the invention of up to 2000%, 1000%, 100%, 95%, 90%, 80%, 75%, 70%, 60%, 50%, 40%, 30%, 20% or 10%.

However, even after swelling, in certain embodiments, the drug-delivery system of the present invention retains its shape or substantial shape over extended periods of time due to crosslinking of the polymer precursors. In certain embodiments, the polymer network of the hydrogel will only substantially degrade after all active agent has been released, or at least after most of the active agent, for example at least 50 wt.-%, 60 wt.-%, 70 wt.-%, 80 wt.-%, 90 wt.-% or 99 wt.-%, or 100 wt.-% of the active agent has been released.

In certain embodiments, it is believed that while swelling in the first place leads to an increase of the size of, e.g., a hydrogel implant, it also has an effect on active agent release and/or the biodegradation of the gel matrix. By swelling of the hydrogel, with increasing amounts of water intruding into the gel, out-diffusion of active agent may be is accelerated, replaced by water.

For example, water diffusing into the gel and/or replacing the organic hydrophobic liquid in the gel over time may steadily dissolve hydrophilic active agents dispersed therein, which can be used to control the active agent release. In this embodiment, the active agent release is mainly or completely controlled diffusion of the active agent into the surrounding tissue. In certain embodiments, another factor influencing or determining the release of the active agent dispersed in the hydrophobic liquid when the active agent release rate is largely independent of the diffusion rate of the hydrophobic liquid, for example when it is an hydrophilic agent dispersed as solid particles in the hydrophobic liquid, is the rate of diffusion of water into the gel and/or hydrophobic liquid, thereby subsequently dissolving and eluting the active agent from the organogel into the surrounding aqueous environment.

Furthermore, in certain embodiments, water slowly diffusing into the hydrogel, which is still crosslinked so it maintains its shape, but swells, so the gel may then be easier (bio-) degraded by hydrolysis and/or enzymatic reactions after the drug-delivery system is depleted of active agent.

The overall release of the active agent is controlled by at least one, or by a combination of all these release mechanisms.

### Delivery System

The sustained drug-delivery system of the present invention may be in the form of an implant, such as a medical implant or a pharmaceutically acceptable implant, an implant coating, or an oral dosage form, etc. The drug-delivery system may also be provided in the form of a kit as further defined herein below, for example for forming an implant in-situ.

The hydrogel drug-delivery system of certain embodiments of the invention can be used for drug delivery to a patient, and for example for ophthalmic drug delivery since it offers a number of advantages as a carrier system. The hydrogel drug-delivery system can be used for drug delivery, gene delivery, antioxidant delivery, peptide delivery, biomedical imaging, and genetic testing in ophthalmology.

If the sustained release, hydrogel drug-delivery system is provided in the form of an implant, the implant may be one of an intraocular implant, intracaveal implant, intracameral implant, an implant for introduction into the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrasceleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.

In embodiments of certain embodiments of the invention, the sustained release drug-delivery system may be formulated for administration via diverse routes such as oral, parenteral, or by operative insertion or injection. Oral dosage forms may consist of the hydrogel of the present invention, which may optionally be enterically coated, or in the form of small particulate forms filled into capsules and the like.

In embodiments where in-situ gel formation is desired, avoiding of pegylation reactions of the active agent with the precursor materials is required, since pegylation or covalent binding of the active agent to the hydrogel precursors interferes with or disturbs in-situ gel formation, and/or show decreased active agent recovery from the hydrogel. For example, hydrogels involving sulfo-DBCO functionalized precursors some times used to improve water solubility may be unsuitable to deliver peptides or proteins, since these agents show strong charge interactions with the sulfonate group that reduces the protein recovery. However, for small molecule API's these hydrogel precursors may be a suitable choice. Further, residual N-hydroxysuccinimide groups on multi-arm precursors not fully functionalized by click chemistry reactive groups may also reduce protein or peptide recovery by covalently binding these agents to hydrogel precursors. In certain embodiments it is preferred to use precursors that form hydrogels wherein the API is dispersed/included in hydrogel, and preferably not covalently bound to the hydrogel or its components.

For in-situ gel forming sustained delivers systems it is preferred to select the precursors to achieve a gel time between 1 to 10 minutes, as further disclosed herein, preferably. 2-4 minutes at 37° C. Such kits or systems may be used in embodiments, particularly for ocular treatments, such as suprachoroidal or intravitreal injections. Depending on the treatment and the injection site involved, target gel times for in-situ intravitreal or blood (IV) may be fast, as defined herein, for suprachoroidal or into body tissue a rather slow gel time is preferred.

In certain embodiments, DBCO/Azide hydrogels may have a gel time too fast for in-situ injections, but may be suitably used for gel formation outside the body to produce implants or inserts, such as plugs.

In other embodiments, Norbornene/Tetrazine based systems may be good for in-situ injections due to relatively long gel times, but without diacid linkers these systems such as the one exemplified in Example 8 are hardly degradable, so that the inclusion of degradable linker are required for sustained release systems.

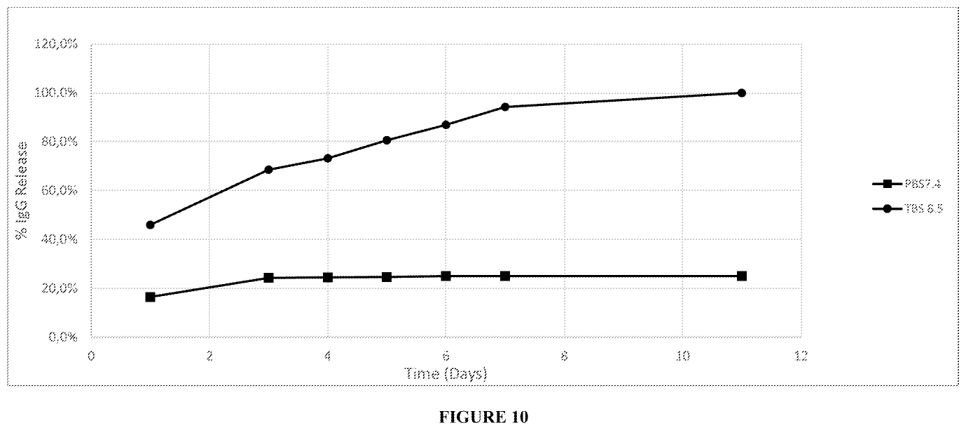

In other embodiments, a DBCO/tetrazine system having gel times of 4-9 min is preferred for in-situ applications, but the gel time thereof is largely independent of temperature and pH. In-vitro data shows that for DBCO/tetrazine PEG hydrogels up to 2 months of degradation time or release of IgG proteins can be achieved, see FIG. 10 and Example 9.

In other embodiments DBCO/azide/tetrazine ternary systems allow controlling and tuning the gel time by the azide precursor concentration, see Example 10.

In certain embodiments, the drug delivery system or kit for in-situ applications has an injectability, determined as described in Example 11, characterized by a glide force at 2 minutes after mixing of less than 10 N, such as less than 9N, 8N, 7N, 6N or less than 5 Newton, and not more than 10N, such as not more than 9N, 8N, 7N, 6N, or 5N.

### Treatment Methods and Administration

According to certain embodiments of the invention, the sustained release drug delivery system comprising the hydrogel is configured for use as a medicament, such as for use in treating a disease or medical condition of a patient.

In an embodiment, the method for treating a disease or medical condition of a patient comprises administering the hydrogel to the patient in order to release the active agent over an extended period of time.

A treatment method of an embodiment of the invention comprises an ocular treatment. In such a treatment, the hydrogel is used to release the active agent over an extended period of time in the eye. In an embodiment thereof, the disease or medical condition to be treated is an eye disease, ocular disease such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy.

The treatment method can also involve treatment of glaucoma, ocular hypertension, hyphema, presbyopia, cataract, retinal vein occlusion, inflammation, myosis, mydriasis, conjunctivitis, intraocular infections, choroidal neovascularization (CNV), intraocular tumors, and retinal neuroinflammation.

The ocular disease may further be one of retinal neovascularization, choroidal neovascularization, Wet AMD, Dry AMD, retinal vein occlusion, diabetic macular edema, retinal degeneration, corneal graft rejection, retinoblastoma, melanoma, glaucoma, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpiginous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angiitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green color blindness.

The methods described in this section can also comprise administration of the hydrogel drug delivery system in combination with another agent, also termed “combination therapy.”

In one embodiment, the combination therapy comprises administering the hydrogel in combination with one or more additional agents either on the same or different day. In one embodiment, the additional agent to be administered in a combination therapy can be a liquid formulation of the agent, or it may be comprised in an oral dosage form. Thus, the additional agent can be any small molecule, large molecule, a protein, a nanoparticle, or any other of the active agents described herein.

The method of ocular treatment of certain embodiments comprising administering the hydrogel may comprise any one of intravitreal, intracameral, subconjunctival, retrobulbar, sub-tenon, subretinal, and suprachoroidal injections. Administration of the hydrogel or drug-delivery system of certain embodiments may also be topical, intradermal, or oral.

In certain embodiments, the drug delivery system is formulated for direct injection at a treatment site of a patient, and may be administered for example by parenteral administration, intratumoral injection, injection into the eye such as intravitreal, intracameral, subconjunctival, retrobulbar, sub-tenon, subretinal, or suprachoroidal injections. In aspects thereof, the drug delivery system is administered by direct injection, by oral application, or incorporated in implants. In certain embodiments, direct injection places a depot and delivers a drug for local or systemic administration. Direct injection may include injecting or implanting a preformed hydrogel at a treatment site, and can further include injection of precursors of the hydrogel or mixtures thereof for in-situ forming an active agent releasing gel depot for delivering a drug for local or systemic administration.

In certain embodiments, the hydrogel or drug-delivery system can be implanted or administered to a body space of a patient by any one of subcutaneously, intramuscularly, intrathecally, epidurally, intraperitoneally, intradermally, subcutaneously, intercostally, intra-articularly, intrasynovially, intraspinally, orally, nasally, rectally, intratumoral, or intravaginal administration. Intra-articular administration is to a joint selected from knee, elbow, hip, sternoclavicular, temporomandibular, carpal, tarsal, wrist, ankle, intervertebral disk, or ligamentum flavum.

The active agent or the additional agent to be administered in a combination therapy, may also be a diagnostic agent. Diagnostic agents have been described above and may be substances used to examine the body in order to detect impairment of its normal functions. In some cases, diagnostic agents may be agents with a functional purpose, such as for use in the detection of ocular deformities, ailments, and pathophysiological aspects.

In certain embodiments of the invention, the sustained release drug delivery system is administered such that the bio-orthogonal hydrogel is formed in-situ at a treatment site of a patient. Since gelation by click chemistry crosslinking as described herein can occur under mild physiologic conditions, and the functional groups do not interfere with physiologic processes, gelation may be done within the patient's body. In aspects thereof, the hydrogel is formed in-situ at a treatment site of a patient by combining, at the treatment site or immediately before, a first formulation comprising a first mixture including a first precursor and an active agent and a second formulation comprising a second mixture including a second precursor and allowing the combined formulation mixture to gel in-situ at the treatment site by click chemistry crosslinking reaction.

In an aspect, the first formulation and the second formulation are combined shortly before administering the formulation mixture at the treatment site. In certain embodiments for treatment by in-situ gelation of the hydrogel, the first formulation is in a first syringe, the second formulation in a second syringe, and the two formulations are combined in a Y-type mixer before direct injection at the treatment site. Alternatively, the first formulation and the second formulation are each administered at the treatment site, for example by separate injections, thereby combining the formulations at the treatment site, and allowing the combined formulations to gel in-situ at the treatment site.

Administration for in-situ gel formation can be done by any one of subcutaneously, intramuscularly, intrathecally, epidurally, intraperitoneally, intradermally, subcutaneously, intercostally, intra-articularly, intrasynovially, intraspinally, orally, nasally, rectally, intratumoral, or intravaginal administration. In certain embodiments thereof, administration for in-situ gel formation example includes parenteral administration, intratumoral injection, injection into the eye such as intravitreal, intracameral, subconjunctival, retrobulbar, sub-tenon, subretinal, or suprachoroidal injections.

### Kit for In-Situ Gel Formation

In an embodiment of the present invention, a kit is provided for forming a bio-orthogonal hydrogel including an active agent. The kit comprises at least two separate containers, such as vials or pre-filled syringes. A first container comprises a first precursor formulation including at least one first multi-arm precursor having first functional groups suitable for forming a connection or crosslink by click chemistry with complementary second functional groups, and optionally at least one active agent and/or at least one solvent. A second container comprises a second precursor formulation including at least one second multi-arm precursor having second functional groups suitable for forming a connection by click chemistry with the first functional groups for mixing and gel formation, and optionally at least one active agent and/or at least one solvent. At least one, such as both, of the first multi-arm precursor or the second multi-arm precursor is a polymer multi-arm precursor having polymer arms comprising polymer units, and at least one of the first multi-arm precursor or the second multi-arm precursor includes a linker unit L comprising a hydrolysable ester bond.

In another embodiment, a third container is provided, wherein the active agent is included separately from the hydrogel precursors, and optionally at least one solvent, and/or the active agent is included in at least one, or both of the first and second containers.

Each of the containers may include additional ingredients such as solvent, pharmaceutical excipients, buffer, additional therapeutic or diagnostic agents, thickeners, adjuvants, and the like. In an aspect of the kit, the first formulation is provided in a first vial or syringe, and the second formulation is provided in a second vial or syringe. The two syringes are formulated for simultaneous or successive injections at a treatment site of the patient. The at least one solvent in the first, second or third container is independently of each other selected from water, aqueous buffered media such as PBS buffer, TBS buffer or saline solutions, or any mixtures thereof, and may optionally include at least one additive selected from pharmaceutical excipients, sugars, salts, additional therapeutic or diagnostic agents, thickeners, viscosity enhancers, stabilizers, or adjuvants.

The kit is suitable for use in a method for preparing an active agent releasing hydrogel in-situ at a treatment site within the body of a patient by simultaneous or successive injections.

In a further aspect, the two syringes of the kit can be connected via a Y-type mixer for combining the two formulations. The kit is suitable for use in a method for preparing an active agent releasing hydrogel in-situ at a treatment site within the body of a patient by a single injection. The kit may also be used for preparing the hydrogel in the form of an extruded strand that may be cut into individual dosage forms outside the body, for forming implant or inserts, such as plugs, for example drug delivering plugs for insertion into the lacrimal canal of the eye.

In another aspect, the first precursor mixture and/or the second precursor mixture may comprise a hydrophobic organic liquid, such as an oil. The first precursor mixture and/or the second precursor mixture may be in the form of a suspension or emulsion and may include the active agent dissolved or suspended therein.

In one aspect, each of the first, second, third or more containers may include a solution of the precursor and/or active agent in a suitable solvent, such as water or buffered aqueous systems. In another aspect, at least one of the first, second, third or more containers includes a solid that may be reconstituted by solvent addition for use. Such embodiments may provide better storage stability. For example, in one embodiment of a kit including three vials or containers, the active agent, such as a peptide or protein, may be a lyophilized API, optionally including sugar buffers such as trehalose. This lyophilized API, upon use of the kit, is first reconstituted into a solution by adding water, buffer or solvent, the solution may then be added to one or both hydrogel precursor solutions, which are subsequently combined to initiate the gelling process.

Sugar buffers may be added as needed to improve solubility and stability of the active agent, such as peptides or proteins, before or after lyophilization. Even with non peptide active agents, the addition of sugar buffers can improve stability and solubility. An exemplary sugar buffer formulation for use with embodiments of the invention may include a solution of sugar, such as trehalose, mono- and diphosphates in water at a suitable concentration, such as 3% (mg/mL) and a pH of about 6.4.

In an embodiment of the kit, at least one first precursor formulation comprises a mixture of two or more first precursors that have each different first functional groups suitable for forming a connection by click chemistry with complementary second functional groups, but not reacting with each other. In another embodiment, the at least one second precursor formulation comprises a mixture of two or more second precursors that have each different second functional groups suitable for forming a connection by click chemistry with complementary first functional groups, but not reacting with each other.

In such embodiments, the different first or second functional groups are selected to have different kinetic rate constants or reaction rates in their click reactions with the corresponding second or first functional group counterparts. This can be used to adjust or tune, i.e. delay or accelerate, the gel time and to adjust the viscosity, i.e. improve injectability, such that in-situ administration for different target sites can be optimized.

The use of the two different second precursors thus provides control over solution viscosity and gel time, which is favorable for different therapeutic applications or administration modes. Very fast gel times (such as about 10 seconds to 2 minutes) can be suitable for in-situ gel forming injections into liquid tissues such as the vitreous humor or blood without the gel getting diluted within the liquid environment. Shorter to moderate gel times (such as about 1 to 3 minutes) may be preferred for injections into soft tissue, and slow gelling (such as about 2 to 8 minutes or more) may be more suitable for in-situ gel forming injections into harder tissue.

In some embodiments, the first and second functional groups are selected to provide a gel time from combining the first and second precursor formulations of at least about 1 minute, such as about 2 minutes or more, or about 3 minutes or more, such as about 1 to 15 minutes, such as about 2 to 10 minutes, 2 to 8 minutes, or 2 to 6 minutes, or 2 to 4 minutes.

In one embodiment, the kit comprises first and second functional groups selected from pairs comprising a strained alkyne moiety such as a dibenzocyclooctyne (DBCO) or a bicyclo[6.1.0]nonyne (BCN), and an azide; or a norbornene or trans-cyclooctene (TCO), and a tetrazine (Tz) moiety; or a strained alkyne moiety such as a dibenzocyclooctyne (DBCO) or a bicyclo[6.1.0]nonyne (BCN), and a tetrazine (Tz) moiety; or any combinations thereof.

In another embodiment, the kit comprises a first precursor being a dibenzocyclooctyne (DBCO) group functionalized multi-arm PEG, and the second precursor is a combination of a tetrazine group functionalized multi-arm PEG and an azide group functionalized multi-arm PEG;

or the first precursor is a combination of a dibenzocyclooctyne (DBCO) group functionalized multi-arm PEG and a norbornene group functionalized multi-arm PEG, and the second precursor is a tetrazine group functionalized multi-arm PEG.

The first and second formulations can be solutions or suspensions of the precursors and optional other components, and may each have a concentration of the polymeric precursors of about 2.5 wt.-% to about 30 wt.-%, such as about 5 to 20 wt.-%, or about 10 wt.-%, based on the total weight of the formulation. In an embodiment, the combined first and second formulations each have a concentration of the polymeric precursors of about 5 wt.-% to about 30 wt.-%, such as about 5 to 25 wt.-%, based on the total weight of the formulation. These concentration ranges can be suitably adapted as desired for achieving sufficient injectability and viscosity, depending on processing means such as needle sizes used.

In certain embodiments, the kit comprises three vials, one including lyophilized bevacizumab (or other active agent) solids, the second vial includes a solution of a multi-arm DBCO functionalized PEG precursor such as 8a15kPEG-DG, and the third vial includes a solution of a mixture of tetrazine-functionalized PEG precursor such as 8a20kPEG-TET, and an azide-functionalized PEG precursor such as 8a20kPEG-N₃. A fourth vial including the solvent for reconstituting the active agent or dissolving/diluting the precursor formulations may also be included in the kit.

Certain aspects of further embodiments of the invention are described in the following sets of embodiments, which are combinable in all aspects with each other.

### First Set of Embodiments

1. A sustained release drug-delivery system, comprising a bio-orthogonal hydrogel and at least one active agent, the bio-orthogonal hydrogel comprising a covalently crosslinked polymer network and containing the at least one active agent, wherein the polymer network comprises
a) a plurality of polymer units,
b) chemical bonds that can be cleaved by hydrolysis, wherein at least one of the hydrolysable bonds is an ester bond, and
c) crosslinks formed by click chemistry reactions.

2. The system of aspect 1, wherein the polymer network comprises multi-arm units having a core unit X and >3 arms, preferably in the range of 3 to 10, or 4 to 8, or 4 to 6, attached to the core unit, wherein a crosslink formed by click chemistry reaction is located on each arm.

3. The system of aspect 2, wherein a crosslink formed by click chemistry reaction is located at the terminus of each arm of the multi-arm units.

4. The system of aspect 2 or 3, wherein the polymer network comprises first, second, and optionally more multi-arm units, wherein the crosslinks formed by click chemistry reactions are between the arms of the first multi-arm units and the second or more multi-arm units.

5. The system of aspect 4, wherein the first multi-arm unit and the second multi-arm unit have the same or a different amount of arms and/or have the same or a different core unit X.

6. The system of aspects 2 to 5, wherein at least one of the multi-arm units is a polymer multi-arm unit having polymer arms comprising polymer units.

7. The system of aspect 6, wherein the polymer multi-arm units have an average molecular weight of 1,000 to 100,000 Daltons.

8. The system according to any of the preceding aspects, wherein the polymer units are selected from at least one of polyethylene glycol (PEG), polyethylene oxide, polypropylene oxide, polypropylene glycol (PPG), poly(ethylene glycol) block poly(propylene glycol) block copolymers, polyvinyl alcohol (PVA), polyvinyl acetate, poly(vinylpyrrolidinone) (PVP), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins.

9. The system according to any of the preceding aspects, wherein the polymer units are selected from at least one of polyethylene glycol (PEG) units, polypropylene glycol (PPG) units, polyglycolic acid (PGA), polylactic acid (PLA), or polylactic-co-glycolic acid (PLGA), random or block copolymers and/or combinations or mixtures of any of these, preferably polyethylene glycol units.

10. The system according to any of the preceding aspects, wherein the wherein the covalently crosslinked polymer network comprises a plurality of hydrophobic polymer units and/or hydrophilic polymer units.

11. The system of aspect 10, wherein the hydrophobic polymer units are selected from at least one of polylactic acid (PLA), and polylactic-co-glycolic acid (PLGA) units.

12. The system of any one of aspects 10 or 11, wherein the hydrophilic polymer units are selected from at least one of polyethylene glycol units, polypropylene glycol units, or polyglycolic acid (PGA), preferably polyethylene glycol units.

13. The system of any one of the preceding aspects, wherein each of the polymer units have an average molecular weight (Mn) in the range from about 1,000 to about 100,000 Daltons, or from about 2,000 to about 50,000 Daltons, or from about 3,000 to about 30,000 Daltons.

14. The system of any one of aspects 4 to 13, wherein the average molecular weight (Mn) of the arms of the first multi-arm unit and the arms of the second multi-arm unit are the same or different.

15. The system of any one of aspects 4 to 14, wherein one of the first or the second multi-arm unit has a molecular weight of less than 1000 Daltons (thereby being a small molecule multi-arm unit) and the other multi-arm unit is a polymer multi-arm unit having an average molecular weight (Mn) of more than 5000 Daltons.

16. The system of any one of aspects 2 to 15, wherein the hydrolysable ester bond is part of a linker unit L being located between the polymer unit terminus of an arm of a multi-arm unit and the crosslinks formed by click chemistry reactions.

17. The system of any one of aspects 4 to 16, wherein the first multi-arm unit and/or the second multi-arm unit include linker units L comprising the hydrolysable ester bond, wherein the linker unit L can be the same or different in the first and second multi-arm unit.

18. The system of any one of aspects 16 or 17, wherein the linker unit L comprises a diacid, or an acid and amide group, such as succinic, glutaric, adipic, azelaic diacids or acid-amides.

19. The system of any one of aspects 16 to 18, wherein the linker unit L comprises a structure represented by Formula (i):

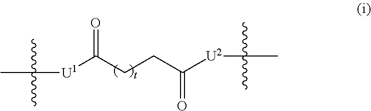

wherein U¹ and U² are independently NH or O and can be the same or different, wherein at least one of U¹ and U² is O, and wherein t is an integer from 0 to 10.

20. The system of any one of aspects 1 to 19, wherein the polymer network comprises multi-arm units represented by formula (ii):

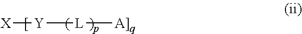

wherein X is the core unit of a multi-arm unit, Y is a polymeric unit, L is a linker unit comprising the hydrolysable ester bond, A is a crosslink formed by a click chemistry reaction, p is either 0 or 1, and q is an integer from 3 to 10.

21. The system of any one of aspects 2 to 15, wherein the core unit X is derivable from a polyol having at least 3 hydroxyl groups, wherein the arms are connected to the hydroxyl groups, or the core unit X is derivable form polyamines having at least 2 amine groups, wherein the arms are connected to the amine groups.

22. The system of any one of aspect 16, wherein the core unit X is derivable from glycerol, pentaerythritol, xylitol, dipentaerythritol, tripentaerythritol, or hexaglycerol, isomalt, lactitol, maltitol, mannitol or sorbitol, preferably pentaerythritol, or derivable from polyamines such as methylene diamine, ethylene diamine, 1,3-diaminopropane, putrescine, cadaverine, hexamethylenediamine, or 1,2-diaminopropane, triamines such as diethylene triamine, propane-1,2,3-triamine, propane-1,1,3-triamine, pentane-1,3,5-triamine, or other polyamines such as triethylene tetramine, butane-1,2,3,4-tetramine, butane-1,1,4,4-tetramine.

23. The system of any one of the preceding aspects, wherein the crosslinks are formed by a click chemistry reaction such as a copper-free SPAAC or IEDDA reaction, wherein a first functional group of a first multi-arm precursor or unit is reacted with at least one complementary, second functional group of a second multi-arm precursor or unit, wherein the second, complementary functional group is capable of reacting with the first functional group in a click chemistry reaction, the first and second functional groups being selected from the pairs comprising an alkyne moiety such as a dibenzocyclooctyne (DBCO) or a bicyclo[6.1.0]nonyne (BCN), and an azide; or a norbornene or trans-cyclooctene (TCO); and a tetrazine (Tz) moiety.

24. The system of any one of the preceding aspects, wherein the crosslinks formed by click chemistry reactions comprise a chemical group selected from triazole and dihydropyridazine.

25. The system of any one of the preceding aspects, wherein the active agent is selected from the group consisting of therapeutically or diagnostically active agents.

26. The system of any one of aspects 1 to 25, wherein the active agent is a small molecule active agent or a biomolecule active agent, preferably a small molecule active agent, a peptide, a protein or a virus.

27. The system of any one of the preceding aspects, wherein the active agent is selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, protein binders such as nanobodies, affibodies, ankyrins, DARPins, etc., or any combinations thereof.

28. The system of any one of the preceding aspects, wherein the active agent is a peptide selected from the group consisting of Compstatin, APL-1, and Fc-III-4C, Beovu 15 (Brolucizumab), Zimura (Avacincaptad Pegol), Pegcetacoplan, Abicipar Pegol, Lampalizumab, Fovista, Risuteganib, AXT107, Elamipretide, THR149, ALM201, VGB3, and Largazole. 29. The system of any one of aspects 1 to 28, wherein the active agent is Bevacizumab.

30. The system of any one of aspects 1 to 29, wherein the active agent is contained in the hydrogel without being covalently conjugated to the hydrogel, or wherein the active agent is contained in the hydrogel and covalently conjugated to the hydrogel.

31. The system of any one of aspects 1 to 30, wherein the active agent is contained in the hydrogel without being covalently conjugated to the hydrogel, preferably, wherein the active agent is contained in the hydrogel without being covalently conjugated to the hydrogel via an oligonucleotide.

32. The system of any one of aspects 1 to 31, wherein the polymer network of the bio-orthogonal hydrogel is formed by reacting a first multi-arm precursor having functional groups comprising an azide (N₃) moiety with a second multi-arm precursor having functional groups comprising a dibenzocyclooctyne (DBCO) moiety.

33. The system of aspect 32, wherein the polymer network of the bio-orthogonal hydrogel is formed by solely reacting a first multi-arm precursor having functional groups comprising an azide (N₃) moiety is with a second multi-arm precursor having functional groups comprising a dibenzocyclooctyne (DBCO) moiety.

34. The system of any one of aspects 1 to 33, wherein the polymer network of the bio-orthogonal hydrogel is formed by reacting a first multi-arm precursor having functional groups comprising an azide (N₃) moiety with a second multi-arm precursor having functional groups comprising a dibenzocyclooctyne (DBCO), and the active agent is a small molecule active agent, a peptide, a protein or a virus.

35. The system of any one of aspects 1 to 33, wherein the polymer network of the bio-orthogonal hydrogel is formed by reacting a first multi-arm precursor having functional groups comprising an azide (N₃) moiety with a second multi-arm precursor having functional groups comprising a dibenzocyclooctyne (DBCO), and the active agent is contained in the hydrogel without being covalently conjugated to the hydrogel, preferably, wherein the active agent is contained in the hydrogel without being covalently conjugated to the hydrogel via an oligonucleotide.

36. The system of any one of aspects 1 to 33, wherein the polymer network of the hydrogel is formed by reacting a first multi-arm precursor having functional groups comprising an azide (N₃) moiety with a second multi-arm precursor having functional groups comprising a dibenzocyclooctyne (DBCO) moiety, and the active agent is contained in the hydrogel without being covalently conjugated to the hydrogel via an oligonucleotide.

37. The system of any one of aspects 1 to 33, wherein the polymer network of the hydrogel is formed by reacting at least one first multi-arm precursor having functional groups comprising an azide (N₃) moiety and/or a tetrazine moiety with a second multi-arm precursor having functional groups comprising a dibenzocyclooctyne (DBCO) moiety, wherein the first multi-arm precursor having functional groups comprising an azide moiety is not derived from pentaerythritol tetrakis(2-bromoisobutyrate), the active agent is a small molecule active agent, a peptide, a protein or a virus and the active agent is contained in the hydrogel without being covalently conjugated to the hydrogel via an oligonucleotide.

38. The system of any one of aspects 32 to 37, wherein the first multi-arm precursor having functional groups comprising an azide (N₃) moiety is selected from the group consisting of:

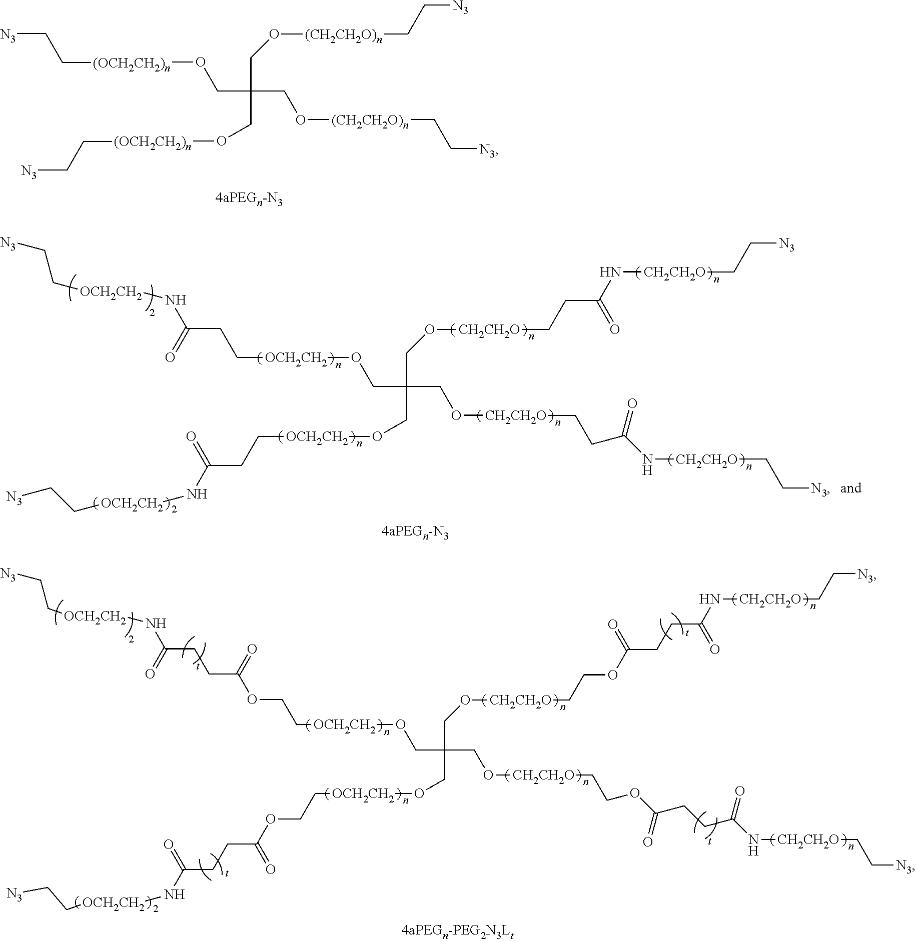

wherein n is 1 to 2300, and t is an integer from 0 to 10, or the corresponding 8 arm structures.

39. The system of any one of aspects 32 to 37, wherein the first multi-arm precursor having functional groups comprising an azide (N₃) moiety is selected from the group consisting of 8a5kPEG-N₃, 8a10kPEG-N₃, 8a15kPEG-N₃, 8a20kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ.

40. The system of any one of aspects 32 to 37, wherein the second multi-arm precursor having functional groups comprising a dibenzocyclooctyne (DBCO) moiety is selected from the group consisting of:

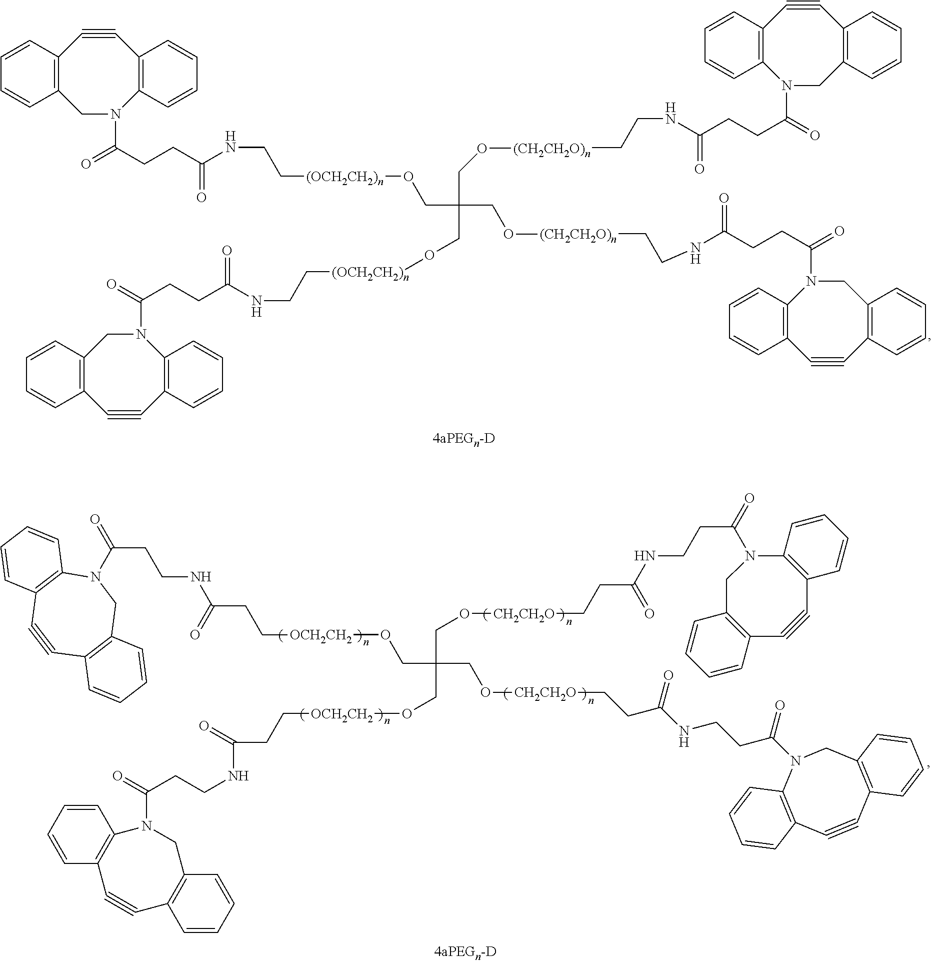

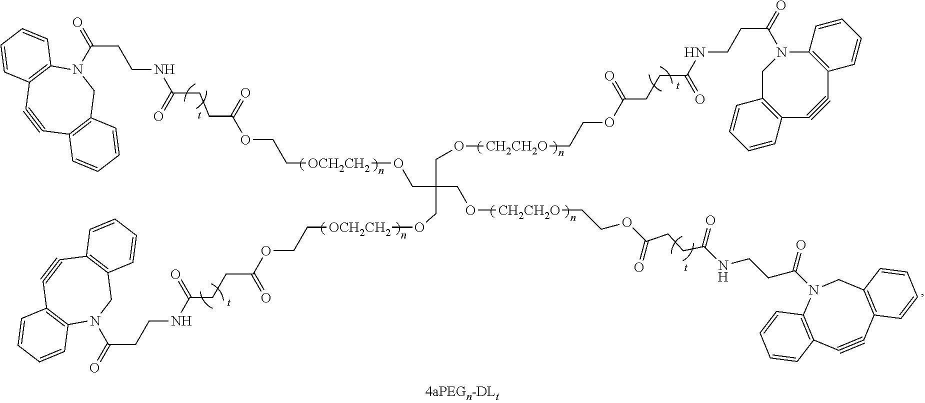

wherein n is 1 to 2300, and t is an integer from 0 to 10, or the corresponding 8 arm structures.

41. The system of any one of aspects 32 to 37 or 40, wherein the second multi-arm precursor having functional groups comprising a dibenzocyclooctyne (DBCO) moiety is selected from the group consisting of 4a20kPEG-D, 6a20kPEG-D, 8a20kPEG-D, 8a15kPEG-DG, 4a20kPEG-DS, 8a10kPEG-DG, 4a20kPEG-DG, 4a20kPEG-DAP, 4a20kPEG-DAZ, 4a20kPEG-DPEG₂₃AZ, and 4a20kPEG-sDG 42. The system of any one of aspects 1 to 37, wherein the polymer network of the bio-orthogonal hydrogel is formed by reacting at least one, preferably one of the following first multi-arm precursors having functional groups comprising an azide (N₃) moiety with at least one, preferably with one of the following second multi-arm precursor having functional groups comprising a dibenzocyclooctyne (DBCO) moiety:

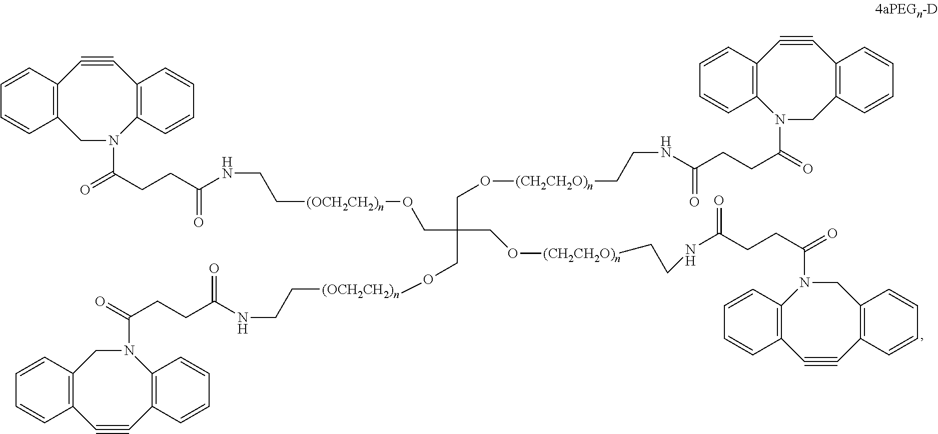

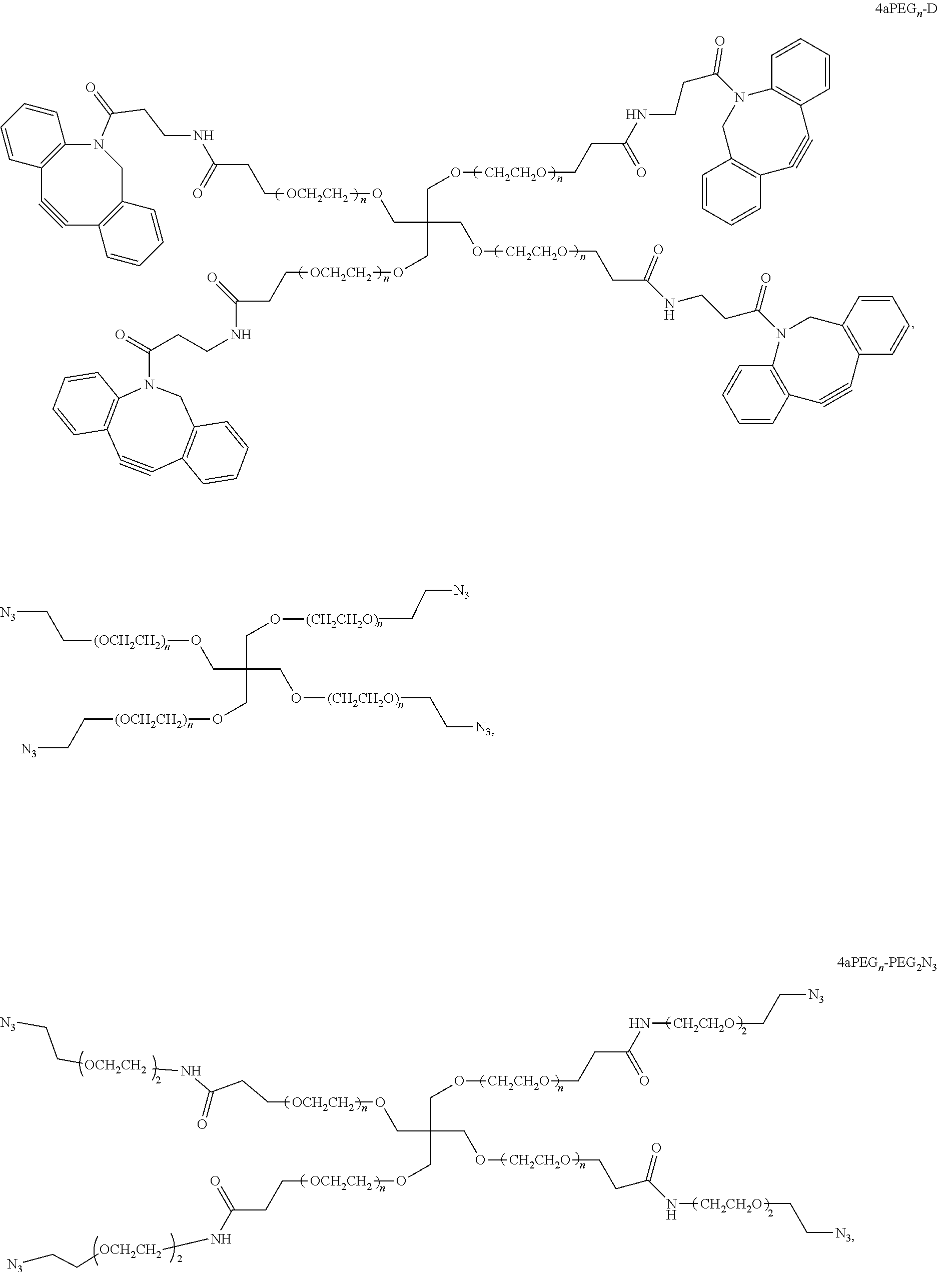

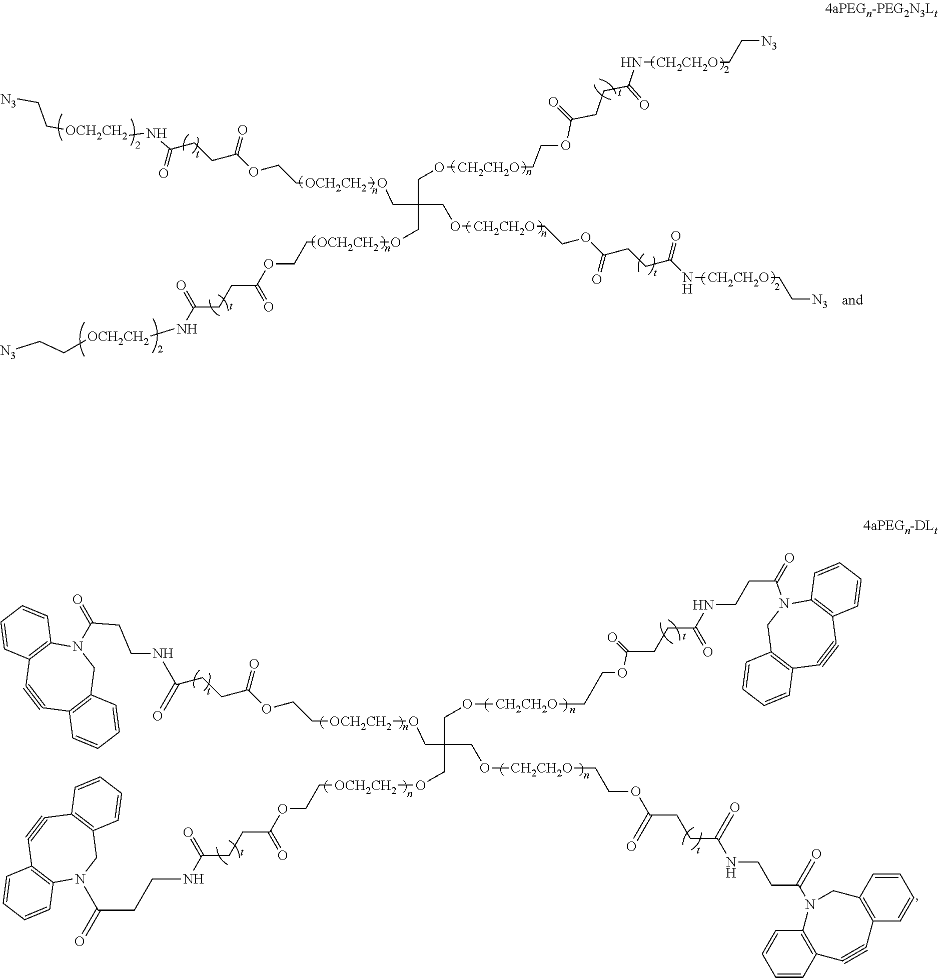

wherein n is 1 to 2300, and t is an integer from 0 to 10, or 8 arm versions of the above, or the corresponding 8 arm structures.

43. The system of any one of aspects 1 to 37 or 42, wherein the polymer network of the bio-orthogonal hydrogel is formed by reacting at least one, preferably one first multi-arm precursor selected from the group consisting of 8a10kPEG-N₃, 4a20kPEG-N₃, 4a20kPEG-PEG₂N₃S, 4a20kPEG-PEG₂N₃G, 4a20kPEG-PEG₂N₃AP, and 4aPEG-PEG₂N₃AZ, 8a20kPEG-Tetrazine, 8a20kPEG-N₃ with at least one, preferably with one second multi-arm precursor selected from the group consisting of 4a20kPEG-D, 6a20kPEG-D, 8a20kPEG-D, 4a20kPEG-DS, 8a10kPEG-DG, 4a20kPEG-DG, 8a15kPEG-DG, 4a20kPEG-DAP, 4a20kPEG-DAZ, 4a20kPEG-DPEG₂₃AZ, 4a20kPEG-sDG and 4a20kPEG-PEG₂BG.

44. The system of any one of aspects 1 to 37, wherein the polymer network of the bio-orthogonal hydrogel is formed by reacting at least one, preferably one of the following first multi-arm precursors having functional groups comprising an azide (N₃) moiety with at least one, preferably with one of the following second multi-arm precursor having functional groups comprising a dibenzocyclooctyne (DBCO) moiety:

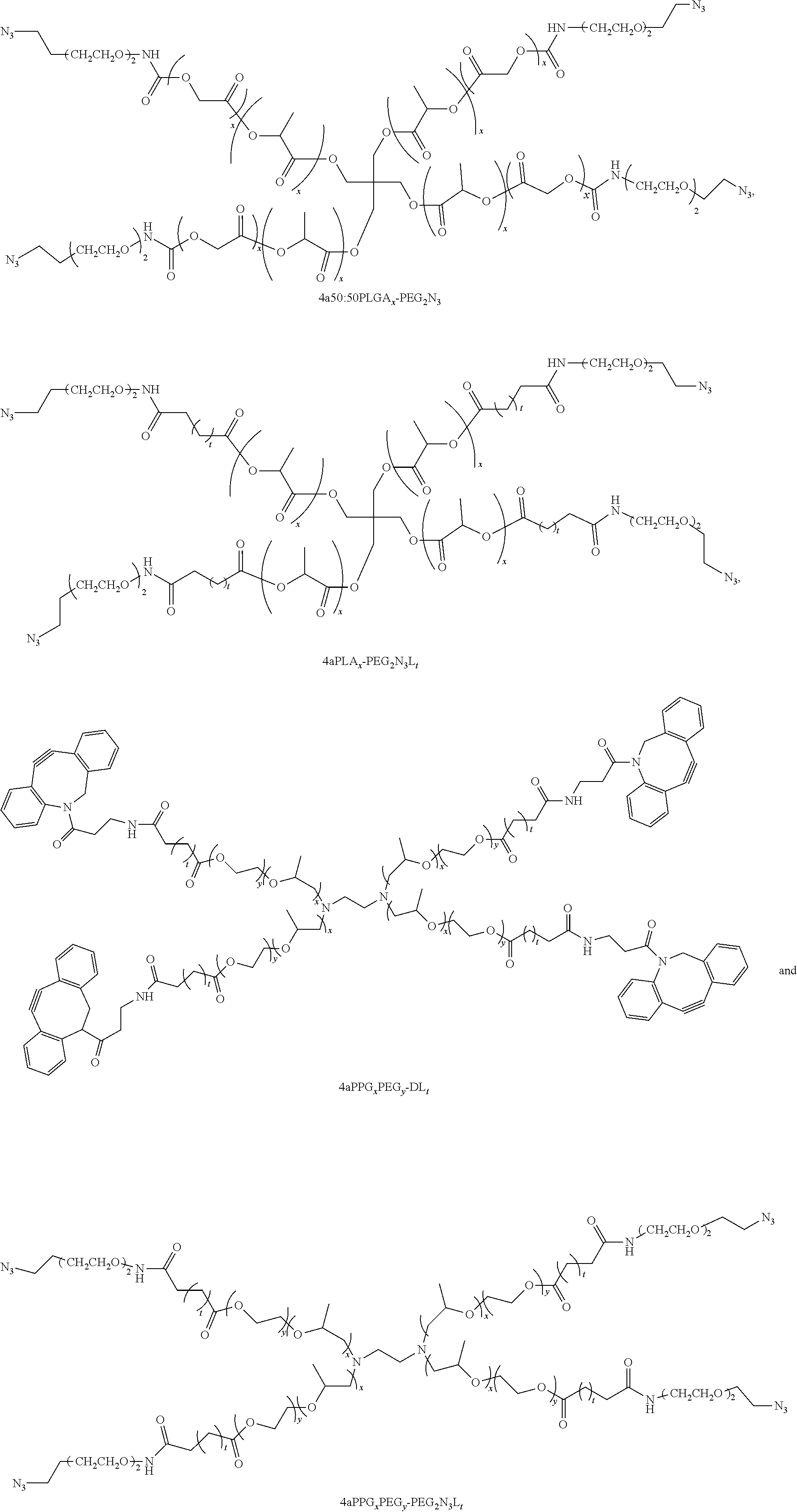

wherein x, y defining the number of PPG, PEG, PLA or PGA units and t is an integer from 0 to 10, or the corresponding 8 arm structures.

45. The system of any one of aspects 1 to 37 or 44, wherein the first multi-arm precursor having functional groups comprising an azide (N₃) moiety is selected from the group consisting of 4a18Tetronic1307-PEG₂N₃, 4a20kPLA-PEG₂N₃, 8a20k50:50PLGA-PEG₂N₃, and 4a20kPLA-PEG₂N₃S.

46. The system of any one of aspects 1 to 37 or 44, wherein the second multi-arm precursor having functional groups comprising a dibenzocyclooctyne (DBCO) moiety is selected from the group consisting of 4a20kPEG-DAZ, 8a20kPEG-D and 4a18Tetronic1307-DAZ.

47. The system of any one of aspects 1 to 37 or 44, wherein the polymer network of the bio-orthogonal hydrogel is formed by reacting at least one, preferably one, first multi-arm precursors having functional groups comprising an azide (N₃) moiety selected from the group consisting of 4a18Tetronic1307-PEG₂N₃, 4a20kPLA-PEG₂N₃, 8a20k50:50PLGA-PEG₂N₃, and 4a20kPLA-PEG₂N₃S, with at least one, preferably with one, second multi-arm precursor having functional groups comprising a dibenzocyclooctyne (DBCO) moiety selected from the group consisting of 4a20kPEG-DAZ, 8a20kPEG-D and 4a18Tetronic1307-DAZ.

48. A method of manufacturing a sustained release drug delivery system as defined in any one of aspects 1 to 47 comprising a bio-orthogonal hydrogel and at least one active agent according to any one of the previous aspects, wherein the method comprises the following steps:
a) preparing a first formulation containing a first multi-arm precursor having first functional groups suitable for forming a connection by click chemistry with second functional groups,
b) providing a second formulation containing a second multi-arm precursor having second functional groups suitable for forming a connection by click chemistry with the first functional groups,
c) combining the first and second formulations,
d) adding at least one active agent to the first, second or combined first and second formulations, thereby forming a combined reaction mixture, and
e) allowing the combined reaction mixture to gel by forming click chemistry connections between the first and second functional groups, thereby forming a hydrogel containing the at least one active agent;
wherein at least one of the first multi-arm precursor or the second multi-arm precursor is a polymer multi-arm precursor having polymer arms comprising polymer units, and
wherein at least one of the first multi-arm precursor or the second multi-arm precursor includes a linker unit L comprising a hydrolysable ester bond.

49. The method of aspect 48, wherein at least one third multi-arm precursor having first or second functional groups suitable for forming click chemistry connections with either the first multi-arm precursor or the second multi-arm precursor is added in one of steps a) to c).

50. The method of aspect 48 or 49, wherein the first and/or second formulation, preferably both, comprises at least one solvent.

51. The method of aspect 50, wherein the at least one solvent is selected from acetone, acetonitrile, benzyl alcohol, chloroform, dichloromethane (DCM), dioxane, dimethyl carbonate (DMC), dimethyl sulfoxide (DMSO), methanol, ethanol or propanol, ethyl acetate, ethyl formate, ethyl propionate, glycofurol, hexafluoro-isopropanol, isosorbide dimethyl ether, isopropanol, methyl chloride, methylene chloride, methyl ethyl ketone, N-methylpyrrolidone, propylene carbonate, triethylamine, or tetrahydrofuran; water, aqueous buffered media such as PBS buffer or saline solutions, sugar buffers; or any mixtures thereof.

52. The method of aspect 51, wherein the at least one solvent is selected from water, aqueous buffered media such as PBS buffer or saline solutions, or sugar buffer; or any mixtures thereof.

53. The method of any one of aspects 48 to 52, further comprising a step of removing the solvent or solvent mixture.

54. The method of aspect 53, wherein removal of the solvent is done by one of nitrogen or other inert gas convection, hot air convection or direct drying, indirect or contact drying, spray drying, dielectric drying, vacuum drying, freeze drying, lyophilization, supercritical or superheated steam drying, or combinations of any of these.

55. The method of any one of aspects 48 to 54, wherein the multi-arm precursors, core and polymer units thereof, linkers L, crosslinks, and active agents are as defined in the system aspects 2 to 47.

56. A sustained release drug-delivery system according to any of aspects 1 to 47, for use as a medicament.

57. A method of treatment, wherein the method comprises treating a disease or medical condition in a patient with a sustained release drug-delivery system according to any one of aspects 1 to 47.

58. The sustained release drug-delivery system for use or the method of treatment of aspects 56 or 57, wherein the sustained release drug-delivery system is used for an ocular treatment.

59. The sustained release drug-delivery system for use or the method of treatment of any one of aspects 56 to 58, wherein the drug-delivery system is used in the treatment of an ocular disease such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy.

60. The sustained release drug-delivery system for use or the method of treatment of any one of aspects 56 to 59, wherein the drug-delivery system is used in the treatment of an ocular disease selected from the group consisting of retinal neovascularisation, choroidal neovascularisation, Wet AMD, Dry AMD, retinal vein occlusion, diabetic macular edema, retinal degeneration, hyphema, presbyopia, corneal graft rejection, retinoblastoma, melanoma, myosis, mydriasis, glaucoma, conjunctivitis, intraocular infections, choroidal neovascularization (CNV), intraocular tumors, retinal neuroinflammation, inflammation, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpiginous choroiditis, subretinal fibrosis, uveitis syndrome, Vogt-Koyanagi Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angiitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked 106 retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green color blindness.

61. The sustained release drug-delivery system for use or the method of treatment of any one of aspects 56 to 60, wherein the drug delivery system is formulated for direct injection at a treatment site of a patient.

62. The sustained release drug-delivery system for use or the method of treatment of aspect 61, wherein the drug delivery system is formulated for parenteral administration, intratumoral injection, injection into the eye such as intravitreal, intracameral, subconjunctival, retrobulbar, sub-tenon, subretinal, or suprachoroidal injections.

63. The sustained release drug-delivery system for use or the method of treatment of any one of aspects 56 to 62, wherein the drug delivery system is administered by direct injection, by oral application, or incorporated in implants.

64. The sustained release drug-delivery system for use or the method of treatment of any one of aspects 56 to 63, wherein the drug-delivery system is administered to a body space of a patient by any one of subcutaneously, intramuscularly, intrathecally, epidurally, intraperitoneally, intradermally, subcutaneously, intercostally, intra-articularly, intrasynovially, intraspinally, orally, nasally, rectally, intratumoral, or intravaginal administration.

65. The sustained release drug-delivery system for use or the method of treatment of aspect 64, wherein intra-articular administration is to a joint selected from knee, elbow, hip, sternoclavicular, temporomandibular, carpal, tarsal, wrist, ankle, intervertebral disk, or ligamentum flavum.

66. The sustained release drug-delivery system for use or the method of treatment of any one of aspects 56 to 65, wherein the drug delivery system comprises two or more different active agents.

67. The sustained release drug-delivery system for use or the method of treatment of aspect 66, for use in a combination therapy involving the administration of more than one active agent.

68. The sustained release drug-delivery system for use or the method of treatment of any one of aspects 56 to 67, wherein the hydrogel of the drug-delivery system is formed in-situ at a treatment site of a patient.

69. The sustained release drug-delivery system for use or the method of treatment of aspect 68, wherein the hydrogel is formed in-situ at a treatment site of a patient by combining a first formulation comprising the first mixture according to step a) of any one of aspects 48 to 55 and a second formulation comprising a second mixture according to step b) of any one of aspects 48 to 55, and allowing the combined formulation mixture to gel in-situ at the treatment site.

70. The sustained release drug-delivery system for use or the method of treatment of aspect 69, wherein the first formulation and the second formulation are combined shortly before administering the formulation mixture at the treatment site.

71. The sustained release drug-delivery system for use or the method of treatment of aspect 70, wherein the first formulation is in a first syringe, the second formulation in a second syringe, and the two formulations are combined in a Y-type mixer before direct injection at the treatment site.

72. The sustained release drug-delivery system for use or the method of treatment of aspect 69, wherein the first formulation and the second formulation are each administered at the treatment site, thereby combining the formulations at the treatment site, and allowing the combined formulations to gel in-situ at the treatment site.

73. The sustained release drug-delivery system for use or the method of treatment of aspect 72, wherein the first formulation is in a first syringe, the second formulation in a second syringe, and the two formulations are injected simultaneously or successively at the treatment site.

74. The sustained release drug-delivery system for use or the method of treatment of aspect 68, wherein the hydrogel is formed in-situ at a treatment site of a patient by administering a suspension comprising a first mixture according to step a) of any of aspects 48 to 55, a second mixture according to step b) of any of aspects 48 to 55 and a hydrophobic organic liquid at the treatment site of the patient.

75. The sustained release drug-delivery system for use or the method of treatment of aspect 74, wherein the hydrophobic organic liquid is included in at least one of the first or second mixture.

76. The sustained release drug-delivery system for use or the method of treatment of aspect 74 or 75, wherein the hydrophobic organic liquid is a biocompatible oil selected from the group consisting of triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyols®), phospholipids, glycerophospholipids, sphingolipids, sterols, prenols, polyketides; hydrophobic biodegradable liquid polymers (such as low molecular weight PLGA, PGA or PLA), low melting point waxes such as plant, animal or synthetic waxes, lanolin, jojoba oil, or combinations thereof.

77. The sustained release drug-delivery system for use or the method of treatment of any one of aspects 74 to 76, wherein the active agent is dissolved or dispersed in the hydrophobic organic liquid; or wherein the active agent is the hydrophobic organic liquid or forms at least a part thereof.

78. A kit for forming a hydrogel as defined in any one of the previous aspects including an active agent as defined in any one of the previous aspects, the kit comprising in separate containers:
a) a first precursor formulation including at least one first multi-arm precursor as defined in any one of the previous aspects having first functional groups suitable for forming a connection by click chemistry with complementary second functional groups, and optionally at least one active agent as defined in any of the previous aspects,
b) a second precursor formulation including at least one second multi-arm precursor as defined in any one of the previous aspects having second functional groups suitable for forming a connection by click chemistry with the first functional groups for mixing and gel formation, and
c) optionally, if not included in a), at least one active agent as defined in any of the previous aspects,
wherein at least one of the first multi-arm precursor or the second multi-arm precursor is a polymer multi-arm precursor having polymer arms comprising polymer units, and wherein at least one of the first multi-arm precursors or the second multi-arm precursors includes a linker unit L comprising a hydrolysable ester bond.

79. The kit of aspect 78, wherein the first formulation is provided in a first syringe, and the second formulation is provided in a second syringe, the syringes being connected via a Y-type mixer for combining the two formulations.

80. The kit of aspect 79, for use in a method for preparing an active agent releasing hydrogel in-situ at a treatment site within the body of a patient.

### Second Set of Embodiments

In one embodiment, the invention is directed to a kit suitable for use for in-situ forming a hydrogel for sustained drug-delivery as follows:

1. A kit suitable for use for in-situ forming a hydrogel for sustained drug-delivery including an active agent, the kit comprising in separate containers:
a) a first container including a first precursor formulation including at least one first multi-arm precursor having first functional groups suitable for forming a connection by click chemistry with complementary second functional groups, and optionally at least one solvent;
b) a second container including a second precursor formulation including at least one second multi-arm precursor having second functional groups suitable for forming a connection by click chemistry with the first functional groups, and optionally at least one solvent; and
c) at least one active agent, included in a separate, third container and/or included in at least one, or both of the first and second containers, and optionally at least one solvent; wherein at least one of the first multi-arm precursor and/or the at least one second multi-arm precursor is a polymer multi-arm precursor having polymer arms comprising polymer units, and wherein at least one of the first multi-arm precursors or the second multi-arm precursors includes a linker unit L comprising a hydrolysable ester bond.

2. The kit of aspect 1, wherein the at least one solvent in the first, second or third container is independently of each other selected from water, aqueous buffered media such as PBS buffer, TBS buffer or saline solutions, or any mixtures thereof, and optionally including at least one additive selected from pharmaceutical excipients, sugars, salts, additional therapeutic or diagnostic agents, thickeners, viscosity enhancers, stabilizers, or adjuvants.

3. The kit of any one of aspect 1 or 2, wherein the at least one first precursor formulation comprises a mixture of two or more first precursors that have each different first functional groups suitable for forming a connection by click chemistry with complementary second functional groups, but not reacting with each other.

4. The kit of any one of aspect 1 or 2, wherein the at least one second precursor formulation comprises a mixture of two or more second precursors that have each different second functional groups suitable for forming a connection by click chemistry with complementary first functional groups, but not reacting with each other.

5. The kit of any one of any one of aspects 3 or 4, wherein the different first or second functional groups are selected to have different kinetic rate constants or reaction rates in their click reactions with the corresponding second or first functional group counterparts.

6. The kit of any one of any one of aspects 1 to 5, wherein the first and second functional groups are selected to provide a gel time from combining the first and second precursor formulations of at least about 1 minute, such as about 2 minutes or more, or about 3 minutes or more, such as about 1 to 15 minutes, such as about 2 to 10 minutes, 2 to 8 minutes, or 2 to 6 minutes, or 2 to 4 minutes.

7. The kit of any one of any one of aspects 1 to 5, wherein the first formulation is provided in a first syringe, and the second formulation is provided in a second syringe, the syringes being connected via a Y-type mixer for combining the two formulations.

8 The kit of any one of any one of aspects 1 to 6, wherein active agent is present as a solid, such as obtained from a lyophilized mixture, that is suitable for reconstitution into an aqueous solution before use.

9. The kit of any one of any one of aspects 1 to 8, wherein the at least one first and at least one second precursors are independently of each other selected from multi-arm polyethylene glycol (PEG) precursors having from 3 to 10 PEG arms, such as or 4 to 8 PEG arms.

10. The kit of any one of any one of aspects 1 to 8, wherein the at least one first and at least one second precursors are independently of each other selected from 3 to 10 arm precursors of polymer units selected from polypropylene glycol (PPG), poly(ethylene glycol)-block-poly(propylene glycol) copolymers, poloxamers such as Tetronic®, polyvinyl alcohol (PVA), polyvinyl acetate, poly(vinylpyrrolidinone) (PVP), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins.

11. The kit of any one of any one of aspects 1 to 10, wherein the first and second multi-arm precursors independently of each other have an average molecular weight (Mn) of 5,000 to 100,000 Daltons, such as 5,000 to 40,000 Daltons, 10,000 to 40,000 Daltons.

12. The kit of any one of the previous aspects, wherein the linker unit L comprises a diacid, or an acid and amide group such as succinic acid, glutaric acid, adipic acid, azelaic acid, or acid amides thereof, such as glutaramide.

13. The kit of any one of the previous aspects, wherein the linker unit L comprises a structure represented by Formula (i):

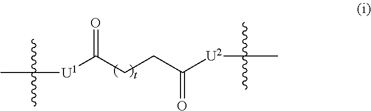

wherein U¹ and U² are independently NH or O, wherein at least one of U¹ and U² is O preferably both are O, and wherein t is an integer from 0 to 10.

14. The kit of any one of any one of the previous aspects, wherein the first and second formulations each have a concentration of the polymeric precursors of about 2.5 wt.-% to about 30 wt.-%, such as about 5 to 20 wt.-%, or about 10 wt.-%, based on the total weight of the formulation.

15. The kit of any one of any one of the previous aspects, wherein the combined first and second formulations each have a concentration of the polymeric precursors of about 5 wt.-% to about 30 wt.-%, such as about 5 to 25 wt.-%, based on the total weight of the formulation.

16. The kit of any one of any one of the previous aspects, wherein the first and second functional groups are selected from pairs comprising a strained alkyne moiety such as a dibenzocyclooctyne (DBCO) or a bicyclo[6.1.0]nonyne (BCN), and an azide; or a norbornene or trans-cyclooctene (TCO), and a tetrazine (Tz) moiety; or a strained alkyne moiety such as a dibenzocyclooctyne (DBCO) or a bicyclo[6.1.0]nonyne (BCN), and a tetrazine (Tz) moiety; or any combinations thereof.

17. The kit of aspect 16, wherein the first precursor is a dibenzocyclooctyne (DBCO) group functionalized multi-arm PEG, and the second precursor is a combination of a tetrazine group functionalized multi-arm PEG and an azide group functionalized multi-arm PEG; or the first precursor is a combination of a dibenzocyclooctyne (DBCO) group functionalized multi-arm PEG and a norbornene group functionalized multi-arm PEG, and the second precursor is a tetrazine group functionalized multi-arm PEG.

18. The kit of any one of the previous aspects, wherein the active agent is included in the hydrogel without being covalently conjugated to the hydrogel, or wherein the active agent is contained in the hydrogel and at least partially covalently conjugated to the hydrogel.

19. The kit of any one of the previous aspects, wherein the active agent is selected from the group consisting of therapeutically or diagnostically active agents such as those defined herein above; or wherein the therapeutically active agent is a small molecule active agent or a biomolecule active agent, preferably a small molecule active agent, a peptide, a protein or a virus, preferably a peptide selected from the group consisting of Compstatin, APL-1, and Fc-III-4C, Beovu 15 (Brolucizumab), Bevacizumab (Avastin), Zimura (Avacincaptad Pegol), Pegcetacoplan, Abicipar Pegol, Lampalizumab, Fovista, Risuteganib, AXT107, Elamipretide, THR149, ALM201, VGB3, and Largazole, preferably Bevacizumab.

20. The kit of any one of aspects 1 to 19, for use in a method for forming an active agent releasing hydrogel in-situ at a treatment site within the body of a patient.

21. A sustained release drug-delivery hydrogel obtainable from the kit of any one of aspects 1 to 19.

### Third Set of Embodiments

In another embodiment, the invention is directed to a method of treatment of a disease or medical condition in a patient by in-situ forming a hydrogel for sustained drug-delivery at a treatment site within the body of a patient as defined below:

1. A method of treatment of a disease or medical condition in a patient by in-situ forming a hydrogel for sustained drug-delivery at a treatment site within the body of a patient, the method comprising:
a) Combining, in the presence of an active agent, a first precursor formulation including at least one solvent and at least one first multi-arm precursor having first functional groups suitable for forming a connection by click chemistry with complementary second functional groups, and optionally, with a second precursor formulation including at least one solvent and at least one second multi-arm precursor having second functional groups suitable for forming a connection by click chemistry with the first functional groups, to form a reaction mixture,
b) Injecting the reaction mixture to a treatment site of the patient, and
c) Allowing the reaction mixture to gel at the treatment site;
wherein at least one of the first multi-arm precursor and/or the at least one second multi-arm precursor is a polymer multi-arm precursor having polymer arms comprising polymer units, and wherein at least one of the first multi-arm precursors or the second multi-arm precursors includes a linker unit L comprising a hydrolysable ester bond.

2. The method of aspect 1, wherein the first and second precursor, first and second functional groups, the active agent, the solvents, and the linker L are as defined in the first or second set of embodiments.

3. The method of aspect 1 or 2, wherein the disease or medical condition is a non-ocular disease, or an ocular disease selected from the group consisting of retinal neovascularisation, choroidal neovascularisation, Wet AMD, Dry AMD, retinal vein occlusion, diabetic macular edema, retinal degeneration, hyphema, presbyopia, corneal graft rejection, retinoblastoma, melanoma, myosis, mydriasis, glaucoma, conjunctivitis, intraocular infections, choroidal neovascularization (CNV), intraocular tumors, retinal neuroinflammation, inflammation, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpiginous choroiditis, subretinal fibrosis, uveitis syndrome, Vogt-Koyanagi Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angiitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked 106 retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green color blindness.

4. The method of any one of the previous aspects, wherein the combined reaction mixture is administered to a body space of a patient for the treatment of ocular diseases by one of intravitreal, intracameral, subconjunctival, retrobulbar, sub-tenon, subretinal, or suprachoroidal injection; or for the treatment of non-ocular diseases by one of subcutaneous, intramuscular, intrathecal, epidural, intraperitoneal, intradermal, subcutaneous, intercostal, intra-articular, intrasynovial, intraspinal, or intratumoral injection.

5. The method of aspect 4, wherein intra-articular administration is to a joint selected from knee, elbow, hip, sternoclavicular, temporomandibular, carpal, tarsal, wrist, ankle, intervertebral disk, or ligamentum flavum.

6. The method of any one of the previous aspects, wherein the kit of any one of aspects 1 to 19 of the first set of embodiments is used.

7. The method of any one of the previous aspects, wherein the first formulation is provided in a first syringe, and the second formulation is provided in a second syringe, the syringes being connected via a Y type mixer for combining the two formulations and directly injecting the reaction mixture.

8. The method of aspect 7, wherein a lyophilized active agent is reconstituted by adding a solvent, and the resulting solution is added to the first syringe, the second syringe, or to both syringes.

9. The method of any one of the previous aspects, wherein the gel time is controlled by using a first precursor formulation comprising a mixture of two or more first precursors that have each different first functional groups suitable for forming a connection by click chemistry with complementary second functional groups, but not reacting with each other, or by using a one second precursor formulation comprising a mixture of two or more second precursors that have each different second functional groups suitable for forming a connection by click chemistry with complementary first functional groups, but not reacting with each other; wherein the different first or second functional groups are selected to have different kinetic rate constants or reaction rates in their click reactions with the corresponding second or first functional group counterparts.

### Fourth Set of Embodiments

In another embodiment, the invention is directed to a hydrogel for sustained drug-delivery obtained by simultaneously gelling multi-arm polymer precursors reactive by two different chemistries, i.e. precursors having functional groups reactive by click chemistry, and having functional groups reactive to crosslink by electrophile-nucleophile reactions, as defined below:

1. A hydrogel for sustained drug-delivery including an active agent, the hydrogel comprising a interpenetrating covalently crosslinked dual polymer network and containing the at least one active agent, wherein the dual polymer network comprises
a) a plurality of polymer units,
b) chemical bonds that can be cleaved by hydrolysis, wherein at least one of the hydrolysable bonds is an ester bond, and
c) crosslinks formed by click chemistry reactions between first and second multi-arm precursors bearing functional groups reactive by click chemistry, and
d) crosslinks formed by electrophile-nucleophile reactions between third and fourth multi-arm precursors bearing functional groups reactive by electrophile-nucleophile reactions.

2. The hydrogel of aspect 1, wherein the functional groups reactive by click chemistry are selected from the from pairs comprising a strained alkyne moiety such as a dibenzocyclooctyne (DBCO) or a bicyclo[6.1.0]nonyne (BCN), and an azide; or a norbornene or trans-cyclooctene (TCO), and a tetrazine (Tz) moiety; or a strained alkyne moiety such as a dibenzocyclooctyne (DBCO) or a bicyclo[6.1.0]nonyne (BCN), and a tetrazine (Tz) moiety; or any combinations thereof.

3. The hydrogel of aspect 1 or 2, wherein the functional groups reactive by electrophile-nucleophile reactions are selected from electrophiles such as activated ester groups, such as succinimidyl esters, succinimidyl carbonates; nitrophenyl carbonates, aldehydes, ketones, acrylates, acrylamides, maleimides, vinylsulfones, iodoacetamides, alkenes, epoxides, mesylates, tosylates, tresyls, cyanurates, orthopyridyl disulfides, or halogen; and nucleophiles such as an amine, such as a primary amine, a hydroxyl, an alcohol, a thiol, an azide anion, and a carboxyl group; preferably from amine and N-hydroxy succinimide groups.

4. The hydrogel of any one of the previous aspects, wherein the dual polymer network is formed by reacting a first multi-arm precursor having functional groups comprising an azide (N₃) and/or tetrazine (Tz) moiety with a second multi-arm precursor having functional groups comprising a dibenzocyclooctyne (DBCO) or Norbornene (Nor) moiety, in the presence of a third precursor having terminal amine (NH₂) groups with a fourth multi-arm precursor having terminal N-hydroxy succinimide (NHS) groups.

5. The hydrogel of any one of the previous aspects, wherein the first to fourth precursors are independently of each other selected from multi-arm polyethylene glycol (PEG) precursors having from 3 to 10 PEG arms, such as or 4 to 8 PEG arms.

6. The hydrogel of any one of the previous aspects, wherein the first to fourth precursors are independently of each other selected from 3 to 10 arm precursors of polymer units selected from polypropylene glycol (PPG), poly(ethylene glycol)-block-poly(propylene glycol) copolymers, poloxamers such as Tetronic®, polyvinyl alcohol (PVA), polyvinyl acetate, poly(vinylpyrrolidinone) (PVP), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins.

7. The hydrogel of any one of the previous aspects, wherein the first to fourth multi-arm precursors independently of each other an average molecular weight (Mn) of 5,000 to 100,000 Daltons, such as 5,000 to 40,000 Daltons, 10,000 to 40,000 Daltons.

8 The hydrogel of any one of the previous aspects, wherein the dual polymer network comprises linker units L comprising a diacid, or an acid and amide group such as succinic acid, glutaric acid, adipic acid, azelaic acid, or acid amides thereof, such as glutaramide.

9. The hydrogel of any one of the previous aspects, wherein the dual polymer network comprises linker units L comprising a structure represented by Formula (i):

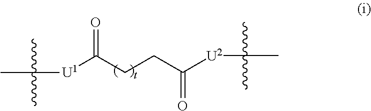

wherein U¹ and U² are independently NH or O, wherein at least one of U¹ and U² is O, preferably both are O, and wherein t is an integer from 0 to 10.

10. The hydrogel of any one of the previous aspects, wherein the active agent is included in the hydrogel without being covalently conjugated to the hydrogel, or wherein the active agent is contained in the hydrogel and at least partially covalently conjugated to the hydrogel.

11. The hydrogel of any one of the previous aspects, wherein the active agent is selected from the group consisting of therapeutically or diagnostically active agents such as those defined herein above; or wherein the therapeutically active agent is a small molecule active agent or a biomolecule active agent, preferably a small molecule active agent, a peptide, a protein or a virus, preferably a peptide selected from the group consisting of Compstatin, APL-1, and Fc-III-4C, Beovu 15 (Brolucizumab), Bevacizumab (Avastin), Zimura (Avacincaptad Pegol), Pegcetacoplan, Abicipar Pegol, Lampalizumab, Fovista, Risuteganib, AXT107, Elamipretide, THR149, ALM201, VGB3, and Largazole, preferably Bevacizumab.

12. The hydrogel of any one of aspects 1 to 11, formed in-situ at a treatment site within the body of a patient.

13. A method of forming the hydrogel of any one of aspects 1 to 11, comprising the following steps:
a) preparing a first formulation comprising a first multi-arm precursor having first functional groups suitable for forming a connection by click chemistry with second functional groups, and at least one active agent as defined in any of the previous aspects,
b) providing a second formulation comprising a second multi-arm precursor having second functional groups suitable for forming a connection by click chemistry with the first functional groups,
c) preparing a third formulation comprising a third multi-arm precursor having third functional groups suitable for forming a connection by electrophile-nucleophile reactions with fourth functional groups,
d) providing a fourth formulation comprising a fourth multi-arm precursor having fourth functional groups suitable for forming a connection by electrophile-nucleophile reactions with the third functional groups, e combining the first to fourth formulations, in the presence of at least one active agent, thereby forming a combined reaction mixture; and
f) allowing the combined reaction mixture to gel by forming click chemistry crosslinks between the first and second functional groups, and by forming crosslinks by electrophile-nucleophile reactions between the third and fourth functional groups, thereby forming a hydrogel containing the at least one active agent;
wherein the first to fourth multi-arm precursors have polymer arms comprising polymer units, and
wherein at least one of the first to fourth multi-arm precursors includes a linker unit L comprising a hydrolysable ester bond.

14. The method of aspect 13, wherein the first to fourth functional groups are selected such that the first and second functional groups are not reactive with either the third or fourth functional group.

15. The method of aspect 13 or 14, wherein the first to fourth formulations further comprise at least one solvent selected from acetone, acetonitrile, benzyl alcohol, chloroform, dichloromethane (DCM), dioxane, dimethyl carbonate (DMC), dimethyl sulfoxide (DMSO), methanol, ethanol or propanol, ethyl acetate, ethyl formate, ethyl propionate, glycofurol, hexafluoro-isopropanol, isosorbide dimethyl ether, isopropanol, methyl chloride, methylene chloride, methyl ethyl ketone, N-methylpyrrolidone, propylene carbonate, triethylamine, or tetrahydrofuran; water, aqueous buffered media such as PBS buffer or saline solutions, sugar buffers; or any mixtures thereof.

16. The method of aspect 15, wherein the at least one solvent is selected from water, aqueous buffered media such as PBS buffer, TBS buffer or saline solutions, or any mixtures thereof, and optionally including at least one additive selected from pharmaceutical excipients, sugars, salts, additional therapeutic or diagnostic agents, thickeners, viscosity enhancers, stabilizers, or adjuvants.

17. A method od any one of aspects 1 to 16, wherein the combined reaction mixture is injected into a treatment site of a patient before gelling is completed.

18. A sustained release drug-delivery system comprising the hydrogel of any one of aspects 1 to 12.

19. A hydrogel for sustained drug delivery, obtainable from the method of any one of aspects 13 to 17.

20. A method for adjusting the gel time of a combined reaction mixture produced in the method of any one of aspects 13 to 17, wherein the functional groups reactive in click chemistry and the functional groups reactive in electrophile-nucleophile reactions are selected to have different kinetics rate constants or reaction rates.

21. The method of aspect 20, wherein the reaction rate of the electrophile-nucleophile reaction is modified by pH adjustment in the combined reaction mixture.

It is understood that the various embodiments described herein are by way of example only and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

### EXAMPLES

### Materials and Methods

Non-degradable 4a20kPEG-N₃, 4a20kPEG-D, Tetronic 1307-N₃, were purchased from creative PEGworks. DBCO-NH₂, sDBCO-NH₂, BCN-PEG₂-NH₂, norbornene-NHS, tetrazine-NHS, tetrazine-PEGs-NHS and N₃-PEG₂-NH₂ (click chemistry linkers) were purchased from Broadpharm, and are also commercially available from Sigma-Aldrich, Sichem, Sinopeg, TCI and other vendors. All the degradable (including di-acid or carboxamide linker) NHS and NH₂ terminated PEGs, PLAs and PLGAs were purchased from Jenkem USA.

TABLE 1
Precursors and designation

| PEG | | | | |
|---|---|---|---|---|
| Molecular | Number | Hydrolysable | Reactive | |
| weight | of | linker | end- | |
| (Da) | arms | moiety L | group | Designation |
| 5000 | 8 | — | Azide | 8a5kPEG-N₃ |
| 10000 | 8 | — | Azide | 8a10kPEG-N₃ |
| 15000 | 8 | — | Azide | 8a15kPEG-N₃ |
| 20000 | 8 | — | Azide | 8a20kPEG-N₃ |
| 20000 | 8 | — | Norbornene | 8a20kPEG-Nor |
| 20000 | 8 | — | Tetrazine | 8a20kPEG-TET |
| 20000 | 8 | — | PEG₅- | 8a20kPEG-PEG₅TET |
| | | | Tetrazine | |
| 20000 | 8 | — | Amine | 8a20kPEG-NH₂ |
| 20000 | 4 | — | Azide | 4a20kPEG-N₃ |
| 20000 | 4 | — | DBCO | 4a20kPEG-D |
| 20000 | 6 | — | DBCO | 6a20kPEG-D |
| 20000 | 8 | — | DBCO | 8a20kPEG-D |
| 10000 | 8 | — | NH₂ | 8a10kPEG-NH₂ |
| 20000 | 4 | Succinic acid | NHS | 4a20kPEG-NHSS |
| 20000 | 4 | Succinic acid | DBCO | 4a20kPEG-DS |
| 20000 | 4 | Succinic acid | Azide | 4a20kPEG-PEG₂N₃S |
| 20000 | 4 | Glutaric acid | NHS | 4a20kPEG-NHSG |
| 10000 | 8 | Glutaric acid | NHS | 8a10kPEG-NHSG |
| 15000 | 8 | Glutaric acid | NHS | 8a15kPEG-NHSG |
| 10000 | 8 | Glutaric acid | DBCO | 8a10kPEG-DG |
| 15000 | 8 | Glutaric acid | DBCO | 8a15kPEG-DG |
| 20000 | 4 | Glutaric acid | DBCO | 4a20kPEG-DG |
| 20000 | 4 | Glutaric acid | Azide | 4a20kPEG-PEG₂N₃G |
| 20000 | 4 | Adipic acid | DBCO | 4a20kPEG-DAP |
| 20000 | 4 | Azelaic acid | DBCO | 4a20kPEG-DAZ |
| 20000 | 4 | Adipic acid | Azide | 4a20kPEG-PEG₂N₃AP |
| 20000 | 4 | Azelaic acid | Azide | 4a20kPEG-PEG₂N₃AZ |
| 20000 | 4 | Azelaic acid | DBCO- | 4a20kPEG-DPEG₂₃AZ |
| | | | PEG₂₃ | |
| 20000 | 4 | Glutaric acid | Sulfo-DBCO | 4a20kPEG-sDG |
| 20000 | | Glutaric acid | BCN-PEG₂ | 4a20kPEG-PEG₂BG |

Phosphate Buffer Solution (PBS) buffer 7.4 buffer is prepared by diluting 200 mL of 10x PBS with RODI to a final volume of 2 mL and adding 200 mg of NaF and 600 μL of Tween 20, then then stirring for 24 hours.

Tris-HCl buffer solution (TBS) 8.5 buffer is prepared by diluting 100 mL of 1 M Tris-HCl solution to 50 mM and then storing it at 37° C. overnight. Once the temperature equilibrates, 200 mg of NaF is added and then returned to the 37° C. incubator for 1 hour to equilibrate. A 600-μL of Tween is added and the container is left to equilibrate for 1 hour at 37° C. again. The pH was adjusted to 8.5 with 6 N NaOH.

### ¹H-NMR Analysis of NHS Conversion to DBCOs and Azides:

Chemical shifts of the different protons in the PEG structures are used to determine the degree of NHS substitution (%-conversion). The degree of NHS substitution in the multi-arm PEG is determined by the ratios of molecular weights determined by MALDI to the molecular weight by ¹H-NMR. The MALDI molecular weight is reported in the certificate of analysis of the purchased PEG material. The ¹HNMR spectra of the converted DBCOs and azides can be used to calculate the %-conversion based on the amide peaks and free NHS peaks.

### HPLC Analysis of DBCO Converted PEGs:

To further understand the purity of the converted DBCO material, an HPLC method was developed to use DBCO-Amine as a standard to quantify the amount of DBCO in the system. The degree of conversion can be calculated based on the remaining concentration of unreacted DBCO-Amine and the purity of the material can be calculated by using the area of the different peaks corresponding to the different substituted PEGs. A Waters UPLC system is used in this analysis with a PDA (photodiode array) detector.

### Example 1

### Preparation of Degradable Polymer Multi-Arm Precursors

The preparations of the degradable polymer multi-arm precursors were done by the following general method. To create a degradable PEG multi-arm precursor terminated with DBCO groups, multi-arm PEGs with NHSS (Succinimidyl Succinate), NHSG (Succinimidyl Glutarate), NHSAP (Succinimidyl Adipate), or NHSAZ (Succinimidyl Azelate) NHS terminal groups are converted into DS (Dibenzocyclooctyne Amido Succinate), DG (Dibenzocyclooctyne Amido Glutarate), DAP (Dibenzocyclooctyne Amido Adipate), or DAZ (Dibenzocyclooctyne Amido Azelate) groups by reacting the NHS group with a DBCO-Amine click chemistry linker according to the following exemplary reaction scheme:

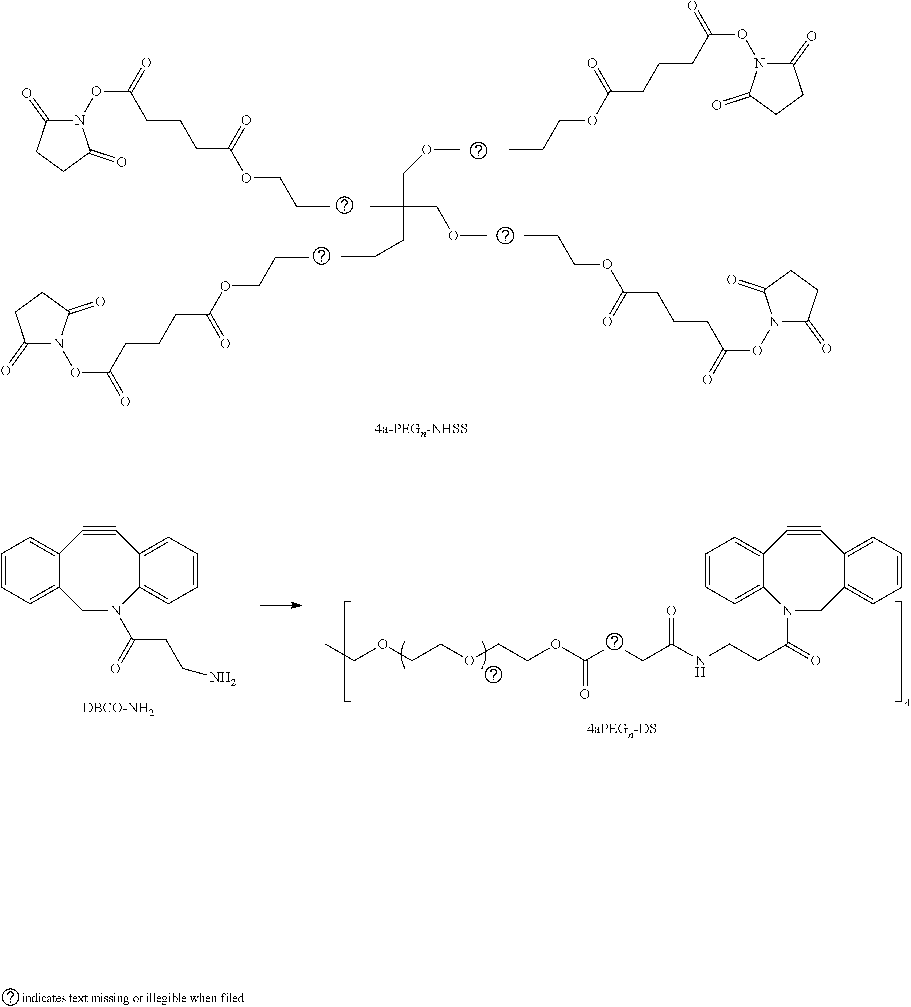

5.5 mg of DBCO-Amine (commercially available from Sigma-Aldrich, TCI and other vendors) are required to ensure a 1:1 reaction with all the NHS groups in a 100 mg of 4a20kPEG-NHSS. Both solids are added to a 20-mL vial and 15 mL of dichloromethane or other suitable solvent) is added. The mixture is vortexed for 1 hour and left to dry under nitrogen overnight. The stoichiometry is calculated (for a 20k PEG precursor) as follows:

$$\frac{100 mg 4 a 20 kPEG - NHSS}{20 , 000 g / mol PEG} \times 4 arms \times \frac{276.3 g}{mol} DBCO - Amine = 5.5 mg DBCO - Amine$$

Similarly, to create a degradable PEG multi-arm precursor terminated with azide groups, a reaction of the 4a20kPEG-NHSS with N₃-PEG₂NH₂ (commercially available from Sigma-Aldrich, TCI and other vendors) is done by weighing 100 mg of 4a20kPEG-NHSS with 3.5 mg of N₃-PEG₂-NH₂ for a 1:1 reaction of the amine groups of the linker and the NHS groups terminating the PEG according to the following exemplary reaction scheme:

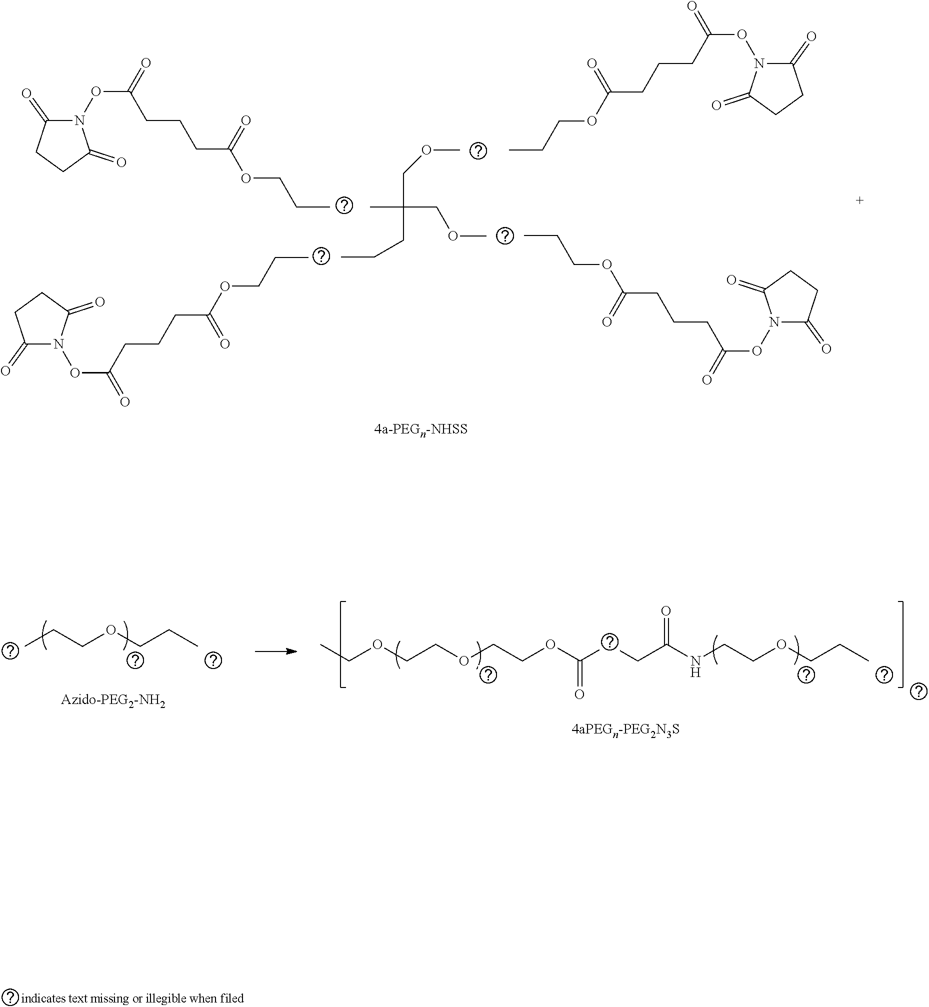

For a 4a20kPEG-NHSS starting material, the stoichiometry is calculated as follows.

$$\frac{100 mg 4 a 20 kPEG - NHSS}{20 , 000 g / mol PEG} \times 4 arms \times \frac{174.2 g Azido - {PEG}_{2} - Amine}{mol} = 3.5 mg Azido - {PEG}_{2} - Amine$$

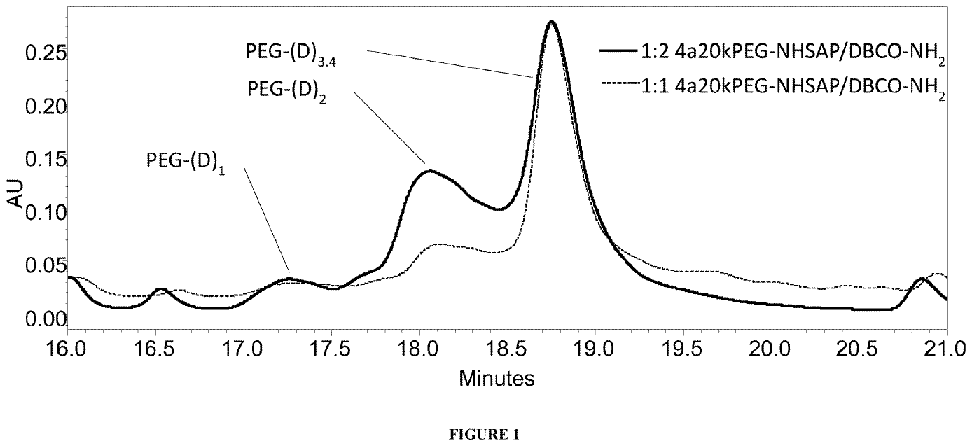

In the same way, other multi-arm PEGs with different molecular weight, arm numbers and other NHS- or NH₂-terminated degradable linker groups can be converted to have terminal DBCO or Azide groups. Characterization and purification of the click chemistry functionalized precursors can be done by ¹H-NMR and HPLC. FIG. 1 shows a HPLC analysis diagram of the product distribution of two reactions having a molar ratio of 1:1 and 1:2 of 4a20kPEGSAP and DBCO-NH₂. A 1:1 stoichiometry leads to significantly less byproducts.

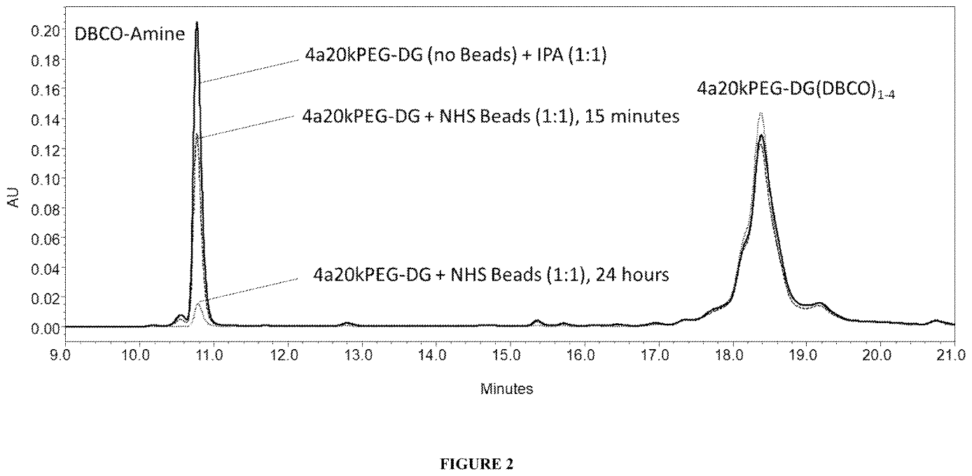

NHS functionalized magnetic beads may be used to remove residual, unreacted DBCO-NH₂ from the products. A corresponding HPLC analysis is shown in FIG. 2. Adding the NHS functionalized magnetic beads for a period of 24 hours and extracting the beads with a magnet efficiently removes unreacted DBCO-NH₂. Based on ¹H-NMR data, conversion of 80-90% and more of the functional groups could be achieved in yet non-optimized reactions. Alternatively or in addition, purification of click functionalized precursors may be done, as required, by centrifugation to remove insoluble impurities, filtration/sterile filtration, and/or dialysis steps as known to the skilled artisan. Precursors and or active agents, or combinations thereof, may be dried and or lyophilized, optionally with the addition of sugar such as trehalose to buffers such as PBS 7.4.

For preparing the hydrogels, the pairs of click chemistry functionalized precursors are combined with mixing, for example from two separate syringes via a Y-type mixer as described herein above.

### Example 2

### Gel Formation and Characterization

For studying gel formation of click chemistry polymer precursors, non-degradable 4a20kPEG-N₃ and 4a20kPEG-D were used. Below are the sections describing the processes associated with formation and characterization of the hydrogels.

### A) Effect of Buffer pH on Gel Formation and Swelling

Solutions of 10 mass-% (50 mg of precursor+450 mg buffer) of the non-degradable 4a20kPEG-N₃ and 4a20kPEG-D in the buffers with pH values summarized in Table 1 were mixed through a syringe mixing process, casted into tubes, and dried overnight under nitrogen. Gel time and 24-hour swell ratios at buffers with pH 7.4-8.5 did not seem to show a significant difference suggesting that buffer pH does not have an impact on the gelation process or the gel quality.

TABLE 1
Effect of buffer pH on gel time and swelling

| | | | Gel | 24-Hour |
|---|---|---|---|---|
| | Mass-% | Mass-% | Time | Swell Ratio |
| Buffer | 4a20kPEG-N₃ | 4a20kPEG-D | (s) | (%) |
| PBS 7.4 | 10 | 10 | 75 | 2461 ± 138 |
| PBS 8.0 | 10 | 10 | 75 | 2405 ± 199 |
| TBS 8.5 | 10 | 10 | 75 | 2709 ± 243 |

Swell ratio is the mass gain after 24 h in buffer divided by the original mass of the gel in %. (% Swell=100*(Mass after 24 h−Original Mass)/Original mass).

### B) Effect of Precursor Concentration on Gel Formation and Swelling

The effect of precursor concentrations in the mixtures to be combined for gel formation was evaluated by syringe mixing of first precursor solutions having 5%, 10%, and 30% by weight 4a20kPEG-N₃ and second precursor solutions having 5%, 10%, and 30% by weight 4a20kPEG-D together and casting the gel in a tube. The results indicate that the onset of gelation and gel quality is strongly dependent on the precursor concentration. Higher precursor concentrations result in much faster gel times with less swelling, suggesting a much tighter polymer network. These findings are summarized in Table 2.

TABLE 2
Effect of %-PEG on gel time and swelling

| Wt .-% precursor in | Mass | Volume PBS | Mass | Volume PBS | Gel | 24-Hour |
|---|---|---|---|---|---|---|
| each of first and | 4a20kPEG-N₃ | 7.4 Buffer for | 4a20kPEG-D | 7.4 Buffer for | Time | Swell |
| second solution | (mg) | Azide (μL) | (mg) | DBCO (μL) | (s) | Ratio (%) |
| 5 | 25 | 475 | 25 | 475 | 260 | 2682 ± 233 |
| 10 | 50 | 450 | 50 | 450 | 90 | 2520 ± 125 |
| 30 | 150 | 350 | 150 | 350 | 25 | 1600 ± 184 |

### C) Effect of Solvent Polarity on Gel Formation and Swelling

Due to the non-polar nature of the DBCO groups, the effect of solvent polarity was investigated to examine whether the solvophillic/solvophobic interactions would impact the gelation process. The results shown in Table 3 indicate that there is a tendency for a longer gel time as the solvent polarity decreases (low dielectric constant).

TABLE 3
Summary of the effect of solvent polarity on gel time

| | | Volume | | | | |
|---|---|---|---|---|---|---|
| | Mass | Azide | Mass | Volume DBCO | Gel | 24-Hour |
| Dielectric | 4a20kPEG-N₃ | Solvent | 4a20kPEG-D | Solvent (μL), | Time | Swell Ratio |
| Constant | (mg) | (μL), Type¹ | (mg) | Type | (s) | (%) |
| 80.1 | 50 | 450, PBS7.4 | 50 | 450, PBS7.4 | 90 | 2520 ± 125 |
| 46.68 | 50 | 450, DMSO | 50 | 450, DMSO | 220 | n.d. |
| 7.85 | 50 | 450, THF | 50 | 450, THF | 330 | n.d. |
| 3.1 | 50 | 450, DMC | 50 | 450, DMC | 270 | 2251 ± 40 |
| 2.38 | 50 | 450, Toluene | 50 | 450, Toluene | 270 | n.d. |

¹DMSO = dimethyl sulfoxide, THF = tetrahydrofuran, DMC = dimethyl carbonate, n.d. = not determined

### D) Effect of Number of Arms (Functionality) on Gel Formation and Swelling

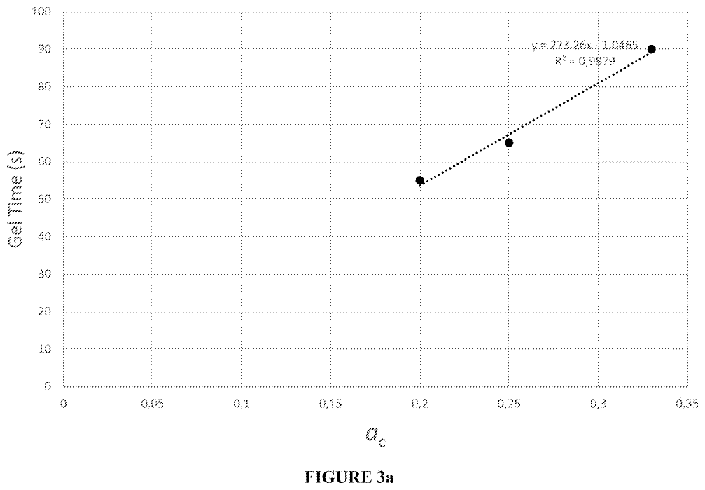

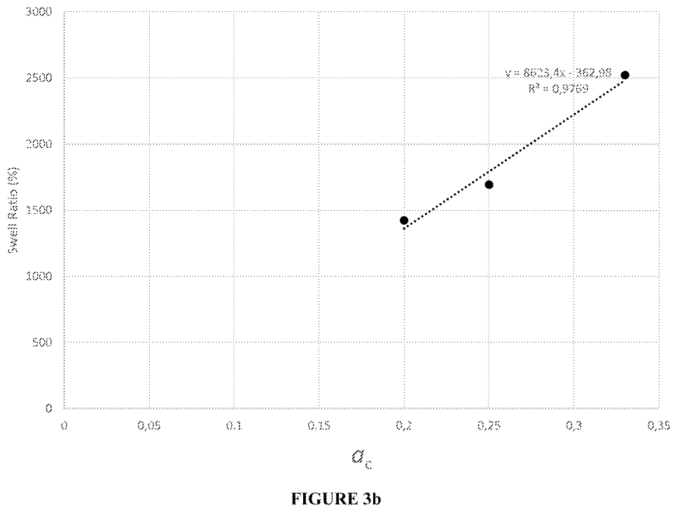

The effect of functionality was evaluated by examining the gelation process of 4a20kPEG-PEG₂N₃ with 4arm, 6arm, and 8arm20kPEG-D. Plotting the obtained gel times and swelling ratios for the 10 wt.-% precursor system obtained by the syringe mixing process against the critical crosslinking value (a_{c}) [(a_{c}=1/(f−1), f is defined as the average number of arms] shows a strong linear dependence where the gel time and swelling ratio that is inversely proportional to the number of arms, see Table 4 and FIG. 3.

TABLE 4
A summary of the effect of the PEG number of arms on the gelation process

| | Mass | Volume Azide | | Volume DBCO | Gel | 24-Hour | |
|---|---|---|---|---|---|---|---|
| # of | 4a20kPEG-N₃ | PBS 7.4 Buffer | Mass PEG-D | PBS 7.4 Buffer | Time | Swell | |
| Arms | (mg) | (μL) | (mg), type | (μL) | (s) | Ratio (%) | α_{c} |
| 4 | 50 | 450.4 | 50, 4a20k | 450 | 90 | 2520 ± 125 | 0.33 |
| 6 | 75 | 550 | 48, 6a20k | 550 | 65 | 1693 ± 124 | 0.25 |
| 8 | 66.6 | 430 | 31, 8a20k | 430 | 55 | 1423 ± 185 | 0.20 |

### E) Effect of Degradable Group on the Gelation Process

Conversion of the NHS PEGs to the corresponding DBCO and azide groups was done according to the methods described earlier. The effect of degradable DBCO vs. degradable azide vs. a double-degradable gel is examined to understand the effect of gel time, swelling, and persistence. Table 5 summarizes the gel times and swelling ratios for the different gels made from polymers with different degradable groups. The results in Table 6 below indicate that the gels formed using a non-degradable azide and degradable DBCO persist longer than gels formed using a non-degradable DBCO and degradable azide as well as the double-degradable gels. Interestingly, the persistence of the NHS-NH₂ gels that were used as a control in this study was the same as the persistence of the double degradable DBCO-Azide gels as well as the non-degradable azide/degradable DBCO gels. Gels containing DS and AS degraded within 24 hours; thus, no swell measurement was taken.

TABLE 5
Summary of the gel times and swelling ratios for the different gels made from PEG
polymers with different degradable groups

| | Mass | Volume PBS | Mass | Volume PBS | Gel | 24-Hour |
|---|---|---|---|---|---|---|
| End Groups | 4a20kPEG-N₃ | 7.4 Buffer for | 4a20kPEG-D | 7.4 Buffer for | Time | Swell |
| used | (mg) | Azide (μL) | (mg) | DBCO (μL) | (s) | Ratio (%) |
| N₃ + DS | 50 | 450 | 50 | 450 | 60 | n.d. |
| N₃ + DG | 50 | 450 | 50 | 450 | 55 | 3240 |
| N₃ + DAP | 50 | 450 | 50 | 450 | 55 | 3000 |
| N₃ + DAZ | 50 | 450 | 50 | 450 | 50 | 2257 |
| PEG₂N₃S + D | 50 | 450 | 50 | 450 | 58 | n.d. |
| PEG₂N₃G + D | 50 | 450 | 50 | 450 | 52 | 4910 |
| PEG₂N₃AP + D | 50 | 450 | 50 | 450 | 55 | 2971 |
| PEG₂N₃AZ + D | 50 | 450 | 50 | 450 | 52 | 1612 |
| PEG₂N₃S + DS | 50 | 450 | 50 | 450 | 60 | n.d. |
| PEG₂N₃G + DG | 50 | 450 | 50 | 450 | 55 | 3336 |
| PEG₂N₃AP + DAP | 50 | 450 | 50 | 450 | 55 | 3069.77 |
| PEG₂N₃AZ + DAZ | 50 | 450 | 50 | 450 | 53 | 2210.23 |

n.d. = not determined

TABLE 6
Persistence comparison of the gels prepared with PEG
polymers with different degradable groups.

| | Day | Day | Day | Day | Day | Day | Day |
|---|---|---|---|---|---|---|---|
| Persistence | 1 | 3 | 4 | 5 | 7 | 8 | 9 |
| N₃ + DG | 1 | 1 | 1 | 3 | 3 | 3 | 3 |
| N₃ + DAP | 1 | 1 | 1 | 1 | 3 | 3 | 3 |
| N₃ + DAZ | 1 | 1 | 1 | 1 | 1 | 1 | 2 |
| PEG₂N₃G + D | 1 | 1 | 3 | 3 | 3 | 3 | 3 |
| PEG₂N₃AP + D | 1 | 1 | 3 | 3 | 3 | 3 | 3 |
| PEG₂N₃AZ + D | 1 | 1 | 1 | 2 | 3 | 3 | 3 |
| PEG₂N₃G + DG | 1 | 1 | 2 | 3 | 3 | 3 | 3 |
| PEG₂N₃AP + DAP | 1 | 1 | 2 | 3 | 3 | 3 | 3 |
| PEG₂N3AZ + DAZ | 1 | 1 | 1 | 1 | 3 | 3 | 3 |
| NH₂ + NHSG | 1 | 1 | 3 | 3 | 3 | 3 | 3 |
| NH₂ + NHSAP | 1 | 1 | 3 | 3 | 3 | 3 | 3 |
| NH₂ + NHSAZ | 1 | 1 | 1 | 2 | 3 | 3 | 3 |

1 \hat{=} Gel still present, 2 \hat{=} Gel present and starting to degrade; 3 \hat{=}Gel completely degraded.

A further look into the chemical structure of these degradable polymers indicates that when a degradable DBCO such as DG, DAP, or DAZ is used, the location of the DBCO group becomes further removed from the degradable ester linkage to the PEG-arm. This may be the reason for the extended persistence compared to using an AS, AG, AAP and AAZ with a non-degradable DBCO. This also explains the similar persistence between the NHS-NH₂ chemistry and the degradable Azide/non-degradable DBCO samples.

### F) Effect of Click Chemistry Linkers on Gel Time, Swelling and Degradation Persistence

Hydrogels have been prepared similarly as described in Examples 1 and 2, using different click chemistry linkers. Precursors were used in about equimolar amounts. It can be seen that longer gel time chemistry is providing a lower swell ratio. Using a more hydrophilic sulfo-DBCO (sD) functional group instead of DBCO or BCN-PEG₂ instead of DBCO increases the persistence in TBS 8.5, and may be used to tailor degradation kinetics. Pushing the DBCO group further away from the linker L (AZ) of precursor 1 by using DBCO-PEG₂₃-NH₂ as a click chemistry linker increases the gel time and reduces the persistence compared to the same DBCO-Amine functionalized polymer. Table 7 summarizes the results.

TABLE 7

| | | Total mass | | | Persistence |
|---|---|---|---|---|---|
| | | in reaction | Gel | Swell | in TBS |
| | | mixture | time | ratio | 8.5 |
| Precursor 1 | Precursor 2 | [%] | [s] | [%] | [days} |
| 4a20kPEG-DG | 4a20kPEG-N₃ | 10 | 61 | 1613 | 3 |
| 4a20kPEG-sDG | 4a20kPEG-N₃ | 10 | 151 | 1458 | 7 |
| 4a20kPEG-PEG₂BG | 4a20kPEG-N₃ | 10 | 506-712 | 1366 | 5 |
| 4a20kPEG-DAZ | 4a20kPEG-N₃ | 20 | 90 | 2265 | 10 |
| 4a20kPEG-PEG₂₃DAZ | 4a20kPEG-N₃ | 20 | (24 hours) | 2785 | 5 |

(% Swell = 100*(Mass after 24 h-Original Mass)/Original mass)

### Example 3

Gels with Polymeric Units Other than PEG

Further to hydrogels with PEG precursors only, hydrogels using DBCO-Azide click chemistry for multi-arm precursors having polymeric units derived from polylactic acid (PLA), poly(lactic-co-glycolic acid) PLGA and poly(ethylene glycol) block poly(propylene glycol) block copolymers (Poloxamer® or Tetronic® copolymers) have been prepared.

Exemplary multi-arm precursors used in this Example are as follows:

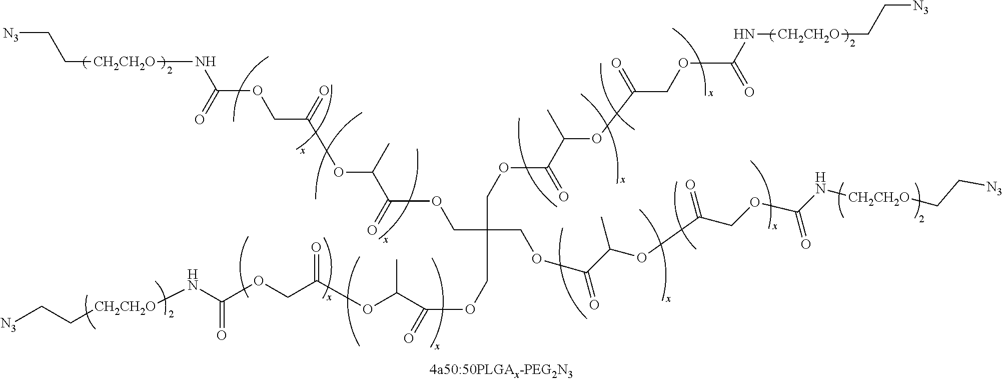

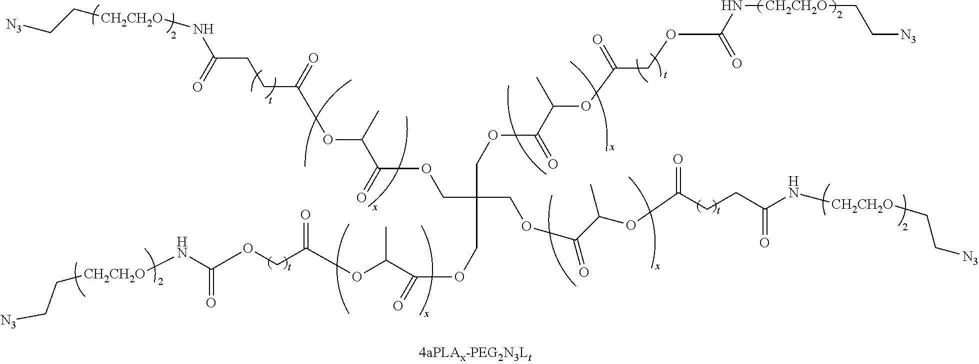

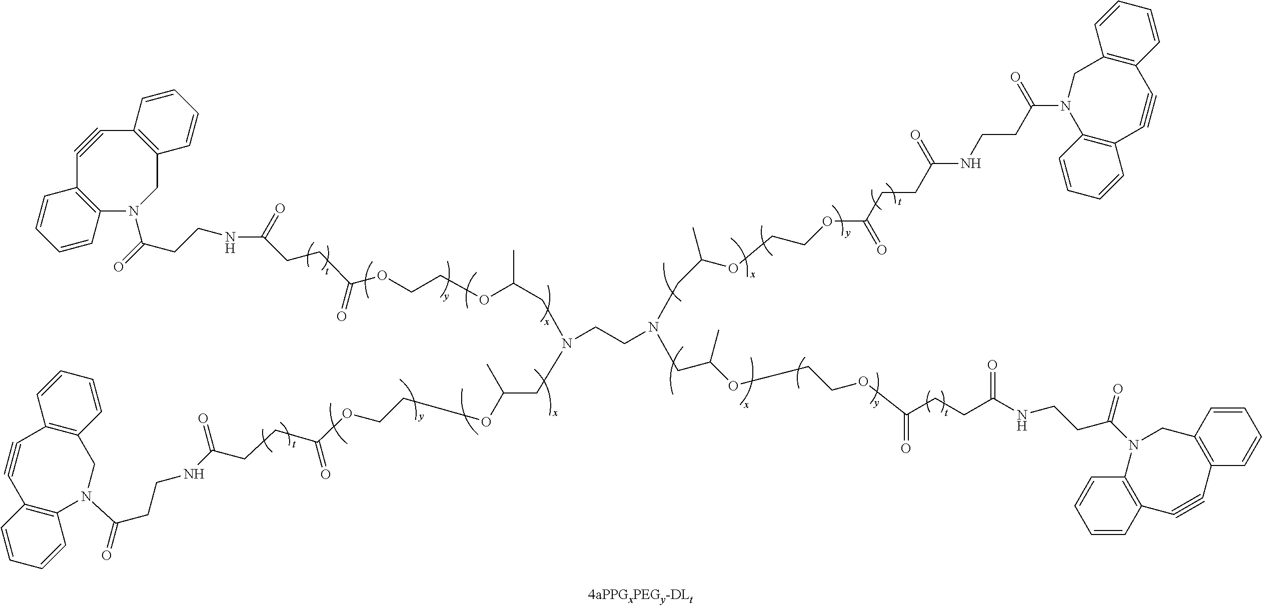

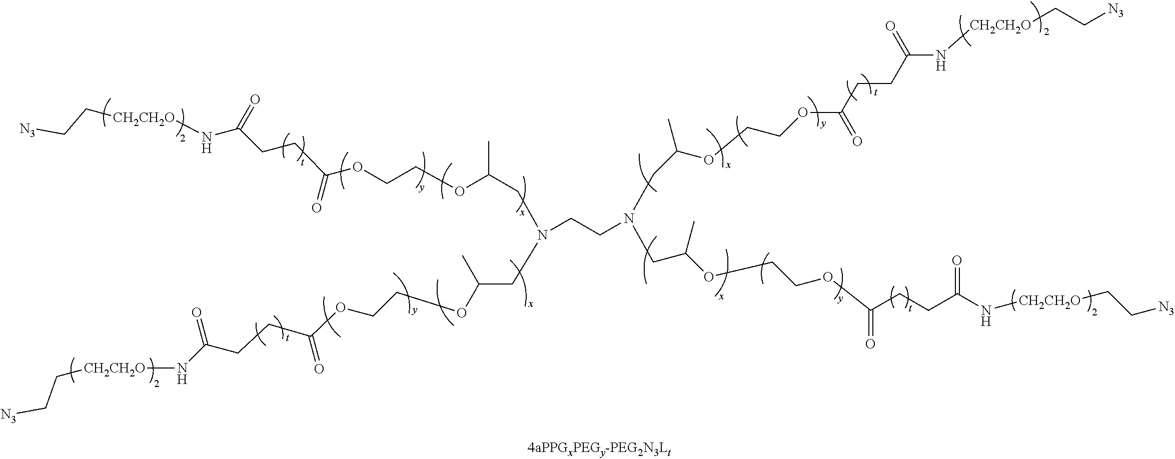

With x, y defining the number of PPG, PEG, PLA or PGA units and t being as defined herein before.

### General Procedure for Preparing the Gels:

Approximately 100 mg of a first polymer precursor (e.g., 4a20k PEG-DAZ) was added to a 1 ml syringe and solvent was added to the same syringe such that the final concentration of polymer in the gel would be according to the Table 8 (about 150 microliter), and the mixture was vortexed at room temperature (20-25° C.). The weight of the second polymer precursor (for e.g., Tetronic 1307-N₃) was adjusted to be molar equivalent required for crosslinking (90.5 mg). Similarly, the required amount of solvent was added to the syringe and vortexed. After complete dissolution, the two syringes were connected by luer lock, and the polymer solutions were mixed for few seconds to start the gelling process. The solution was pushed through one syringe and gel time was measured. The gel was dried for at least 2 days under a nitrogen gas stream at room temperature. The gel was analyzed for swelling by measuring the weight of a gel piece before and after soaking in 1×PBS (pH 7.4) for 24 hours at room temperature. The persistence of the gel was measured by soaking a gel piece in TBS pH 8.5 buffer (tris-buffered saline) at 37° C. and daily visual inspection for degradation of the gel. The solvent used was dimethyl carbonate (DMC):Acetone (80:20). The final concentration of polymer (mg/ml) in solvent after mixing of the two solutions before gelling is given in %. Table 8 shows the precursor combinations used, the gel time, swell ratio and persistence of the obtained gels.

TABLE 8
Gel time, Swell ratio and persistence of Gels with polymeric units selected from PLA,
PLGA, and Tetronic (PPG-PEG).

| | | | Polymer | Gel | Swell | |
|---|---|---|---|---|---|---|
| Gel | | | concentration | time | ratio | Persistence |
| # | Precursor 1 | Precursor 2 | [%] | [min] | [%] | [days] |
| 1 | 4a18kTetronic1307- | 4a20kPEG-DAZ | 15 | 3.33 | 911.89 | 8 |
| | PEG₂N₃ | | | | | |
| 2 | 4a20kPLA-PEG₂N₃ | 4a20kPEG-DAZ | 25 | 65 | 96.1 | 7 |
| 3 | 8a20k50:50PLGA- | 4a20kPEG-DAZ | 25 | 115 | 343.8 | 2 |
| | PEG₂N₃ | | | | | |
| 4 | 4a20kPLA-PEG₂N₃S | 8a20kPEG-D | 20 | <0.17 | 87.8 | >40 |
| 5 | 4a18kTetronic1307- | 4a18kTetronic1307- | 15 | 402 | 598.41 | 7 |
| | PEG₂N₃ | DAZ | | | | |

### Example 4

### Peptide Compatibility of Precursors

To examine whether the DBCO-Azide chemistry is compatible with peptides to be included into the gel as active agents, 8a20kPEG-NHSG, 8a10kPEG-NH₂, 8a20kPEG-DG and 8a10kPEG-PEG₂Na precursors were dissolved at 30 mass-% concentration in a solution containing 9 mass-% lysozyme, a peptide with a MW of 14 KDa, in PBS 7.4. The solutions of the 8a10kPEG-NH₂, 8a10kPEG-PEG₂N₃ and 8a20kPEG-DG showed a clear solution upon dissolving the precursors in the lysozyme solution. However, a small solid, gel-like particle formed while trying to dissolve the 8a20kPEG-NHSG in the lysozyme solution. This suggests that under aqueous conditions, the primary amine groups on the lysozyme undergo nucleophilic reactions with the NHS groups of the 8a20kPEG-NHSG resulting in pegylation and cross-linking with the lysozyme. The precursors functionalized with click chemistry functional groups do not interact with the peptide, thus allowing peptide inclusion into a gel formed therefrom.

### Example 5

### AAV Delivery

Gold nanoparticles have been used as models to study the solution behavior of adeno-associated virus (AAV). Hydrogels can sustain the release of gold nanoparticles having the same size and characteristics as AAV. By varying the formulation, fast release and slow release hydrogel formulations can be created, which can then be tested for AAV delivery.

To evaluate the suitability of hydrogels formed by click-chemistry reactions for the release of AAVs, lyophilized gold nanoparticles were suspended in this example in DMC solutions containing 6% each of PEG precursors of azide and DBCO before gel formation. 4a20kPEG-PEG₂N₃ was used as the azide component and 4a20kPEG-DS, 4a40kPEG-DG were used separately as the DBCO component as well as a 50/50 blend of the 4a20kPEG-DS/4a40kPEG-DG. Gels including dispersed lyophilized gold nanoparticles were produced and analyzed.

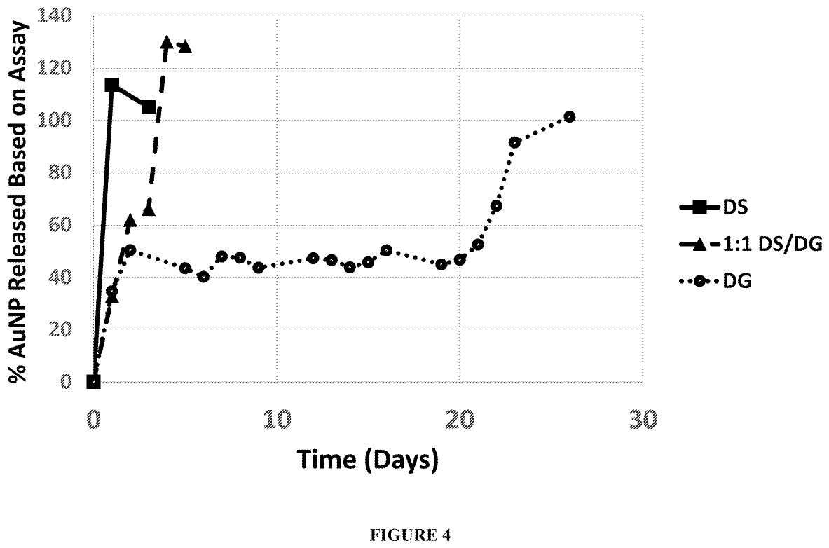

The results indicate that when 100% 4a20kPEG-DG is used as the DBCO component, the gels persists for more than twenty days and there is a two-step delivery approach where a burst of 40% release is done in the first few days, followed by a 15-day plateau, and then another sustained release region until 100% gold nanoparticles are recovered. Interestingly, when the 50/50 blend of 4a20kPEG-DS/4arm40kPEG-DG is used as the DBCO component, the gels persist for 5 days and shows a sustained release profile of gold nanoparticles that is almost linear. On the other hand, the gels degraded overnight when 100% 4armk20PEG-DS was used as the DBCO component. These results show that the DBCO/Azide click chemistry can be used to sustain the release of the gold nanoparticle models that are used for AAVs. Results are summarized in FIG. 4.

### Example 6

### Protein Delivery

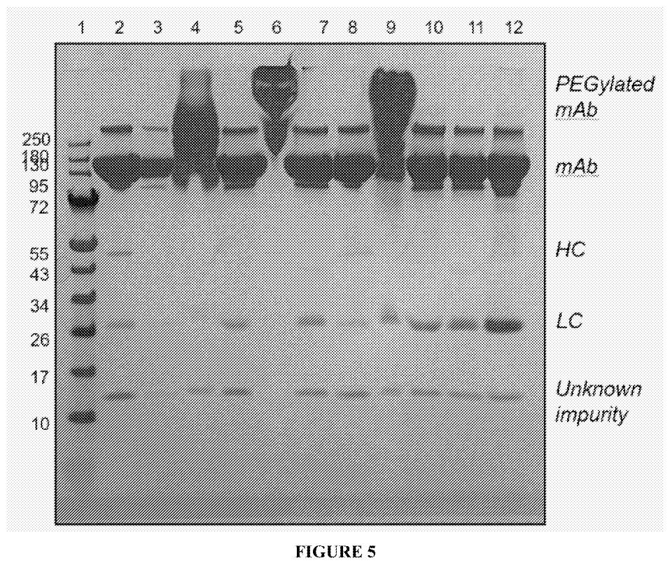

Bevacizumab (Avastin kit) was chosen as the API to test the feasibility of the DBCO/Azide PEG hydrogels in protein delivery. To test this, several PEG precursors containing DBCO, Azide, NHS, and NH₂ functional groups were added to aqueous solutions containing Bevacizumab, and SDS-PAGE were used to test for aggregates. The results shown in FIG. 5 and Table 9 demonstrate that large aggregates, corresponding to pegylated Bevacizumab, only exist when an NHS-containing PEG is added to the Bevacizumab solution. These results suggest that protein delivery through DBCO/Azide-PEG hydrogels can be achieved under aqueous conditions.

TABLE 9
SDS-PAGE lane description of FIG. 5

| Lane | Polymer composition |
|---|---|
| 1 | — |
| 2 | N/A |
| 3 | N/A |
| 4 | m20kPEG-SC* |
| 5 | 8a20kPEG-NH₂ |
| 6 | 8a10kPEG-NHSG |
| 7 | 8a20kPEG-N₃ |
| 8 | 8a10kPEG-D |
| 9 | m20kPEG-SC* |
| 10 | 8a20kPEG-NH₂ |
| 11 | 8a20kPEG-N₃ |
| 12 | 8a20kPEG-D |

*m20kPEG-SC is a linear mPEG20k succinimidyl carboxymethyl ester, 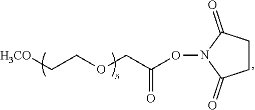
commercially available from Sigma Aldrich.

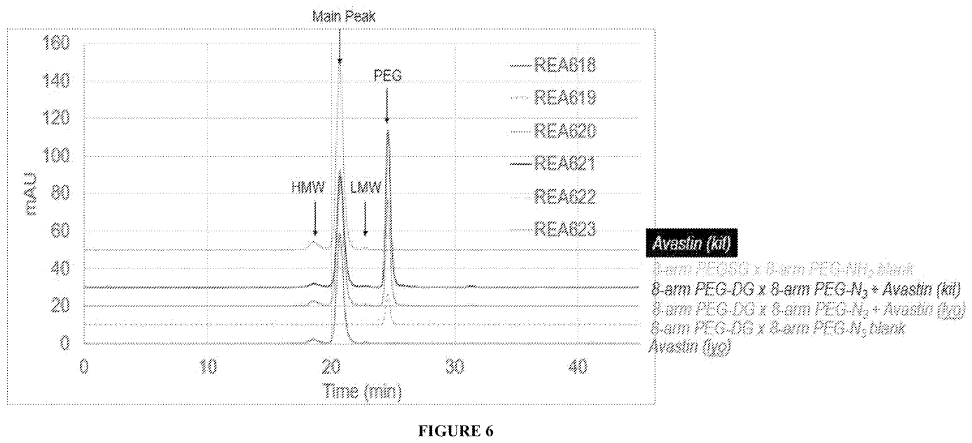

Furthermore, SEC-HPLC analysis of the samples that did not undergo pegylation under gel electrophoresis indicated the presence of low MW and high MW species as well as the main Bevacizumab and the PEG peaks (FIG. 6). The % Bevacizumab recovery was reasonable (80-110%), suggesting that there is no pegylation with azide (N₃), NH₂ and DBCO groups on the PEG.

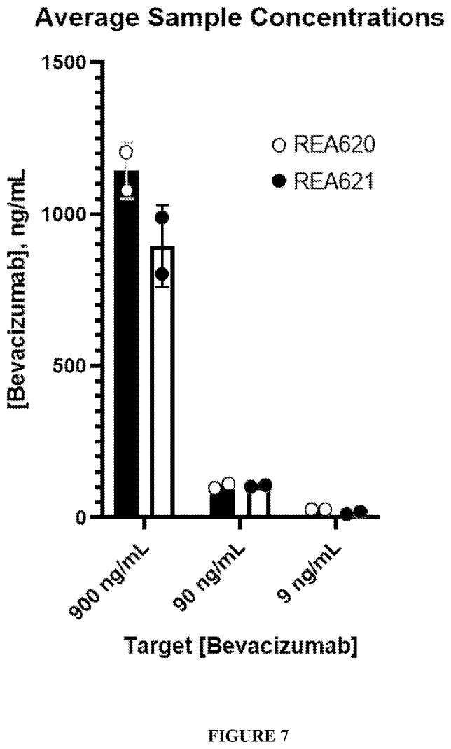

Moreover, ELISA was used to further confirm the concentration Bevacizumab in solution FIG. 7 indicates there is a good %-recovery of bevacizumab from the gels at different concentrations with 900 ng/mL being excellent.

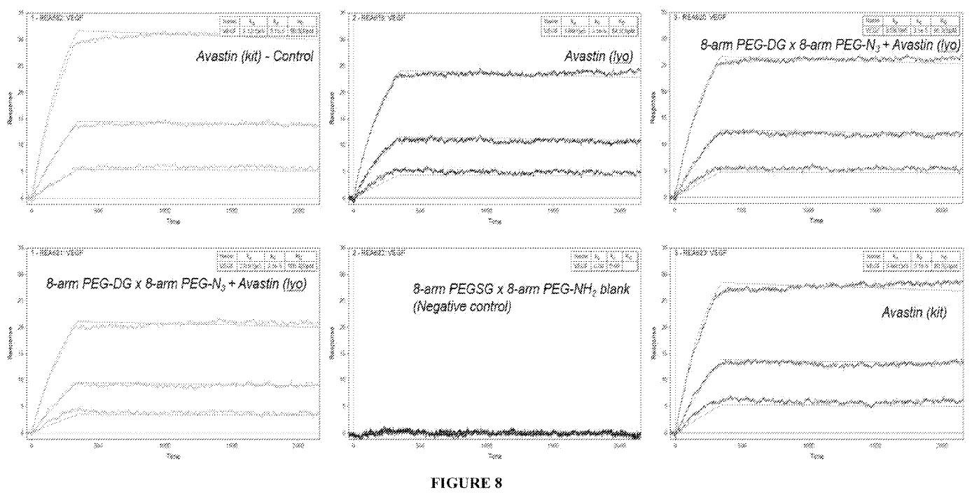

SPR analysis was done to evaluate the protein binding to VEGF. The results summarized in Table 10 and FIG. 8 show that there is no apparent loss of Bevacizumab function by SPR. Theoretical Kps and kas are similar, the mAb capture levels are similar and no binding observed when PEG control is injected.

TABLE 10

| | | VEGF | mAb | | | | | | |
|---|---|---|---|---|---|---|---|---|---|
| | | bind? | capture | kd | k_{d} | K_{D} | Theoretical | Actual | Rₘₐₓ/ |
| Entity | Product name | (y/n) | level | (M⁻¹ s⁻¹) | (s⁻¹)** | (M) | Rₘₐₓ | Rₘₐₓ | R_{maxtb} |
| REA592 | Avastin (kit) | Y | 280 | 3.12E+05 | 3.1E−05 | 9.93E−11 | 72 | 48.1 | 0.67 |
| REA618 | CM-665-020-1 | Y | 200 | 3.66E+05 | 3.1E−05 | 8.48E−11 | 51.5 | 33.6 | 0.65 |
| REA620 | CM-665-020-3 | Y | 225 | 3.59E+06 | 3.1E−05 | 8.63E−11 | 57.9 | 37.6 | 0.65 |
| REA821 | CM-665-020-4 | Y | 184 | 2.91E+05 | 3.1E−05 | 1.06E−10 | 47.4 | 33.4 | 0.70 |
| REA622 | CM-665-020-5 | N* | 3 | N/A | N/A | N/A | N/A | N/A | N/A |
| REA623 | CM-665-020-6 | Y | 235 | 3.86E+06 | 3.1E−05 | 8.03E−11 | 60.5 | 38.6 | 0.64 |

### Example 7

### Effect of Crosslinking Density (MWc) on Release Kinetics

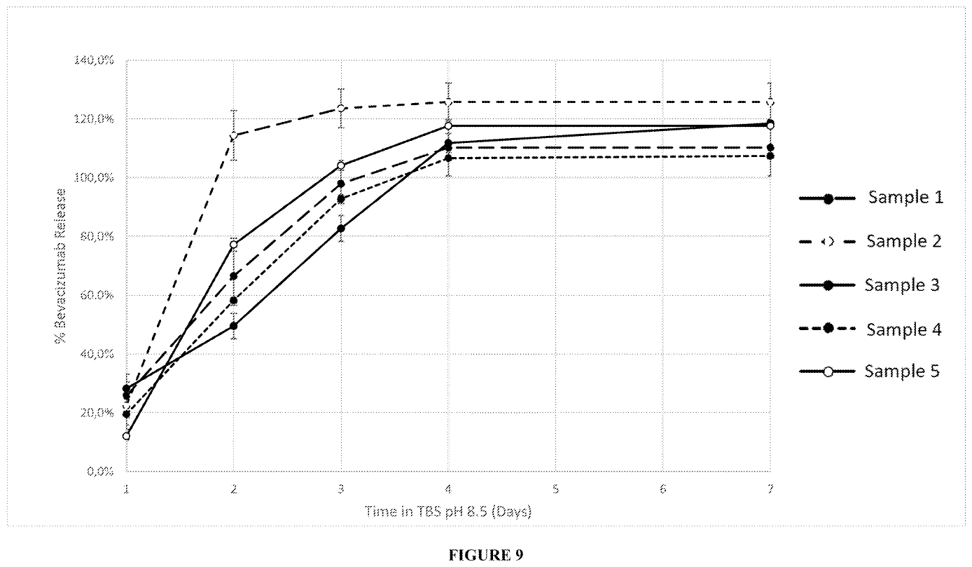

Bevacizumab (Avastin kit) was chosen as the API to test the effect of molecular weight between crosslinks (MWc) on the release kinetics of the API in different hydrogels formed according to the general procedure described in Example 1. Several multi-arm PEG precursors containing DBCO, Azide, NHS, and NH₂ functional groups were added to aqueous solutions containing Bevacizumab, at a weight ratio of total PEG:Avastin of 3:1 and a total PEG precursor concentration in aqueous solution of 10 wt.-%. PEG arm length and number of arms were varied to give different MWc. The compositions are summarized in Table 11, and the release kinetics are shown in FIG. 9 as % Bevacizumab release, normalized to assay.

TABLE 11

| Sample | | | MWc | Precursor:Avastin | % Precursor |
|---|---|---|---|---|---|
| # | Precursor 1 | Precursor 2 | (Da) | total solids | (w/w) |
| 1 | 4a20kPEG-DG | 8a5kPEG-N₃ | 5625 | 3:1 | 10 |
| 2 | 8a15kPEG-DG | 8a20kPEG-N₃ | 4375 | 3:1 | 10 |
| 3 | 4a20kPEG-DG | 8a15kPEG-N₃ | 6875 | 3:1 | 10 |
| 4 | 4a20kPEG-DG | 8a20kPEG-N₃ | 7500 | 3:1 | 10 |
| 5 | 4a20kPEG-DG | 4a20kPEG-N₃ | 10000 | 3:1 | 10 |

The release kinetics data in FIG. 9 reveals a control of burst at a MWc of greater than 4375 and a sustained in-vitro release in TBS 8.5 of the API. Higher MWc, as expected delays the API release to 4 days in TBS 8.5 at room temperature, corresponding to about 2 months at 37° C. in PBS 7.4.

### Example 8

### Norbornene/Tetrazine Hydrogels

Following the general procedure described in Example 1, IgG containing hydrogels (at different hydrogel: IgG ratios (PEG: IgG)) where produced from mixing aqueous solutions including equimolar amounts (each at about 10% precursor concentration (% PEG)) of 8a20kPEG-Norbornene (8a20kPEG-Nor, obtained by reacting 8a20kPEG-NH₂ with norbornene-NHS) and (A,B) a tetrazine precursor (8a20kPEG-TET), and (C) a PEGs extended tetrazine precursor (8a20kPEG-PEG₅TET) in the presence of IgG.

The tetrazine precursors were obtained by reacting 8a20kPEG-NH₂ and with either tetrazine-NHS ester (see scheme 8 below), or tetrazine-PEGs-NHS, in DCM for 8 hours, 82% conversion by ¹H-NMR for 8a20kPEG-TET, and 100% conversion by ¹H-NMR for 8a20kPEG-PEGsTET, and evaporating the solvent under nitrogen atmosphere, dissolving in PBS 7.4, centrifugation at 10000 rpm for 30 minutes, decanting, filtering with 0.25 μm filter, dialysis with 10k MWC for 24 hours in WFI 3.8 followed by sterile filtration or lyophilization. The IgG was mixed in equal amounts into the two precursor solutions in two separate syringes, and the two formulations from the two syringes were combined in a Y-type mixer to produce the IgG containing hydrogel.

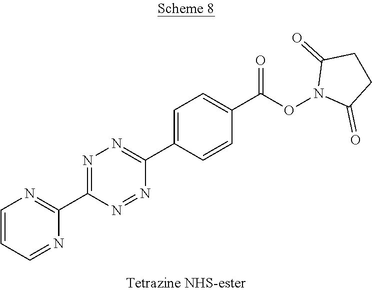 [SMILES: O=C(ON1C(=O)CCC1=O)c1ccc(-c2nnc(-c3ncccn3)nn2)cc1]

Gel time was found to be at 2 to 6 minutes at room temperature (20° C.). The samples each had a calculated molecular weight between crosslinks MWc of about 5000 Da. Release data of IgG released from hydrogel in PBS 7.4 buffer at 37° C. for all samples showed a relatively low initial burst of less than 10% of IgG (% IgG BURST) at different PEG: IgG ratios. No pegylation of precursors to the protein could be determined by SEC. In an accelerated persistence test of 1 week in TBS pH 8.5 buffer no degradation was observed, as expected, as there are no degradable linkers in the precursors. The results are summarized in Table 12:

TABLE 12

| Sample | Precursor 1 | Precursor 2 | % PEG | PEG:IgG | % IgG Burst |
|---|---|---|---|---|---|
| (A) | 8a20kPEG-TET | 8a20kPEG-Nor | 10 | 10:1 | 0% |
| (B) | 8a20kPEG-TET | 8a20kPEG-Nor | 10 | 3.3:1 | 3.79 ± 1.9% |
| (C) | 8a20kPEG-PEG₅TET | 8a20KPEG-Nor | 10 | 10:1 | 9.8 ± 3.4% |

### Example 9

### DBCO/Tetrazine Hydrogels

Following the general procedure described in Example 1, IgG containing hydrogels where produced from mixing aqueous solutions including equimolar amounts of 8a15kPEG-DG and an 8a20kPEG-TET precursor (same as in Example 7 above) in the presence of IgG. The 8a15kPEG-DG was obtained by reacting eight arm 15 kDa PEG succinimidyl glutarate (8a15kPEG-SG) with DBCO-NH₂ in DCM for 8 hours, evaporating the solvent under nitrogen-atmosphere, dissolving in water for injection (WFI 3.8), centrifugation at 10000 rpm for 30 minutes, decanting, filtering with 0.25 μm filter, dialysis with 10k MWC for 24 hours in WFI 3.8 followed by sterile filtration, 94% conversion by ¹H-NMR.

Gel time was found to be at 4.5 (30 wt.-% PEG in precursor solution) to 8.7 minutes (10% PEG in precursor solution) at room temperature (20° C.), suitable for administration by in-situ gel formation. The samples each had a calculated MWc of about 4375 Da. Release data of IgG released from hydrogel in PBS 7.4 and TBS 8.5 buffer at 37° C. showed a relatively low initial burst of IgG (% IgG BURST) in PBS 7.4, and a complete IgG release under accelerated conditions in TBS 8.5 at room temperature within 11 days, showing the suitability for long term drug delivery of the DBCO/tetrazine hydrogels. The release data is shown in FIG. 10.

### Example 10

### DBCO/Tetrazine/Azide Hydrogels

To vary the gel time for in-situ applications, hydrogels were produced using two differently functionalized multi-arm PEG precursors both reacting with the DBCO functionalized multi-arm precursor at different reaction rates. Following the general procedure described in Example 1, 8a20kTET and 8a20kN3 were reacted with 8a15kPEG-DG (see Example 8) at varying amounts of the azide precursor to produce hydrogels.

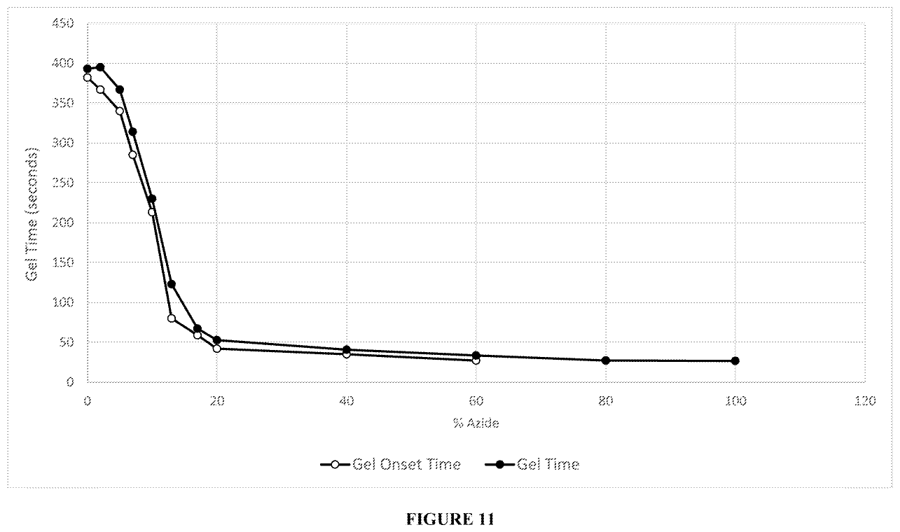

Gel onset and complete gel formation times were measured using the stir bar method, and the results are shown in FIG. 11. The stir bar method involves mixing the two gel precursors together for 10 seconds, then injecting into a vial containing a magnetic stir bar that is stirring and recording the time until the stir bar slows down indicating a higher viscosity. This time point is defined as the “gel onset”. “gel time” is defined as the time point when the stir bar finally stops stirring.

The graph shows that in a concentration range of up to about 20 wt.-% of azide precursor the gel time strongly varies from about 7 minutes at 0 wt.-% azide precursor to about 1 minute at about 20 wt. % azide precursor. An inflection point of the curve at an azide precursor concentration of about 13% for this system results in a gel time of about 3 to 4 minutes gel time, which can be used to adapt the handling period for in-situ gel formation applications.

The use of the two different second precursors thus provides control over solution viscosity and gel time, which is favorable for different therapeutic applications or administration modes. Very fast gel times (such as about 10 seconds to 2 minutes) can be suitable for in-situ gel forming injections into liquid tissues such as the vitreous humor or blood without the gel getting diluted within the liquid environment. Shorter to moderate gel times (such as about 1 to 3 minutes) may be preferred for injections into soft tissue, and slow gelling (such as about 2 to 8 minutes or more) may be more suitable for in-situ gel forming injections into harder tissue.

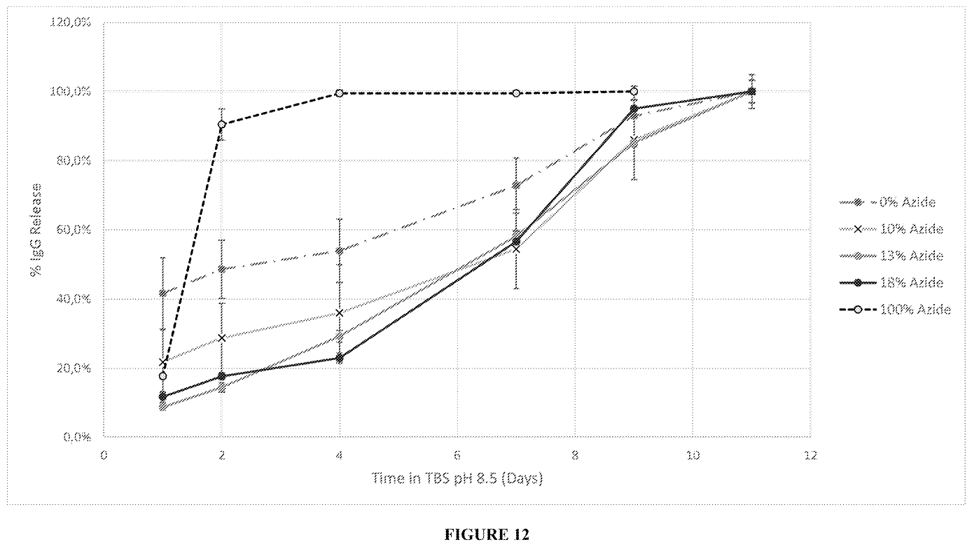

Further, 24 hour swell data in PBS or TBS does not seem to be affected by the Azide concentration, indicating a fully crosslinked structure in each of the gels. Accelerated degradation/IgG in-vitro release kinetics data over time in TBS 8.5 buffer for the gel samples having 0, 10, 13, 18 and 100 wt.-% azide precursor has been obtained and is shown in FIG. 12.

The hydrogel with 100 wt.-% azide precursor (equimolar amounts of 8a20kPEG-N₃/8a15kPEG-DG) shows almost complete burst by day 2. The 0 wt.-% azide hydrogel (equimolar amounts of 8a20kPEG-TET/8a15kPEG-DG shows a high burst on day 1, followed by a constant release up to day 11. Sustained release of the IgG drug over 11 days under accelerated conditions with relatively small initial burst is obtained with the hydrogels having 10 wt.-%, 13 wt.-%, and 18 wt.-% azide precursor (equimolar amounts of 8a15kPEG-DG and the sum of 8a20kPEG-TET and 8a20kPEG-N₃ in a weight-% ratio of 90:10, 87:13 and 82:18). Degradation in TBS 8.5 is about 16 times accelerated compared to PBS 7.4 which resembles in-vivo conditions, so that long term sustained active agent release in-vivo of up to about 6 months can be expected.

### Example 11

### Injectability

An important parameter for application by in-situ gel forming sustained release hydrogels is the injectability of the combined precursor solutions. This parameter can be obtained by determining the glide force to be applied on the filled syringes in an Instron Model 3342 device with a 50N load cell. The setup used is as follows: 100 μl volumes of premixed aqueous precursor solutions (8a15kPEG-DG as in Example 8 in one syringe and a mixture of 95 wt.-% 8a20kPEG-Tetrazine/5 wt.-% 8a20kPEG-N₃ in the other syringe), at a concentration of 10 wt.-% combined precursor content including different concentrations of active agent (bovine IgG, MW about 150 kDa) in a 1 ml syringe having a diameter of 4.78 mm are injected at 1 minutes to 7 minutes every minute at an injection rate of 100 mm/min for an injection distance of 100 mm per 50 μl injection, and the displacement versus applied force is recorded. The glide force at 2 minutes after mixing is taken for comparison of the different gel samples. The results are provided in Table 13.

TABLE 13

| | IgG | | | | | Glide |
|---|---|---|---|---|---|---|
| Sam- | Con- | Instron | Injec- | | | Force |
| ple | cen- | Injection | tion | Mixing | Gel | @ |
| # | tration | Times | Size | Time | Time | 2 min |
| 1 | 2.5 mg/ | 1, 2, 3, | 100 | 10 | 7 min | 9.4 N |
| | 50 μl | 4, 5, 7 | μl | Seconds | | |
| | | min | | | | |
| 2 | 3 mg/ | 1, 2, 3, | | | 7.5 | 7.6 N |
| | 50 μl | 4, 5, 7 | | | min | |
| | | min | | | | |
| 3 | 4 mg/ | 1, 2, 3, | | | 5.5 | 7.7 N |
| | 50 μl | 4, 5, 6 | | | min | |
| | | min | | | | |
| 4 | 5 mg/ | 1, 2, 3, | | | 4 | 15.7 N |
| | 50 μl | 4 min | | | min | |

The results show, that for the system tested in this example, a n active agent dose of 2.5 to 4 mg in 50 μl injection volume appears to be ideal for injectability.

1-91. (canceled)

92. A method of treatment of a disease or medical condition in a patient, the method comprising administering a sustained release drug-delivery system comprising a bio-orthogonal hydrogel and at least one active agent, the bio-orthogonal hydrogel comprising a covalently crosslinked polymer network and containing the at least one active agent, wherein the polymer network comprises
a) a plurality of polymer units,
b) chemical bonds that can be cleaved by hydrolysis, wherein at least one of the hydrolysable bonds is an ester bond, and
c) crosslinks formed by click chemistry reactions.

93. The method of claim 92, wherein the sustained release drug-delivery system is used for an ocular treatment.

94. The method of claim 92, wherein the drug-delivery system is used in the treatment of an ocular disease such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy.

95. The method of claim 92, wherein the drug-delivery system is used in the treatment of an ocular disease selected from the group consisting of retinal neovascularisation, choroidal neovascularisation, Wet AMD, Dry AMD, retinal vein occlusion, diabetic macular edema, retinal degeneration, hyphema, presbyopia, corneal graft rejection, retinoblastoma, melanoma, myosis, mydriasis, glaucoma, conjunctivitis, intraocular infections, choroidal neovascularization (CNV), intraocular tumors, retinal neuroinflammation, inflammation, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpiginous choroiditis, subretinal fibrosis, uveitis syndrome, Vogt-Koyanagi Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angiitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked 106 retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green color blindness.

96. The method of claim 92, wherein the drug delivery system is formulated for direct injection at a treatment site of a patient, preferably the drug delivery system is formulated for parenteral administration, intratumoral injection, injection into the eye such as intravitreal, intracameral, subconjunctival, retrobulbar, sub-tenon, subretinal, or suprachoroidal injections.

97. The method of claim 92, wherein the drug delivery system is administered by direct injection, by oral application, or is incorporated in implants.

98. The method of claim 92, wherein the drug-delivery system is administered to a body space of a patient by any one of subcutaneously, intramuscularly, intrathecally, epidurally, intraperitoneally, intradermally, subcutaneously, intercostally, intra-articularly, intrasynovially, intraspinally, orally, nasally, rectally, intratumoral, or intravaginal administration.

99. The method of claim 98, wherein intra-articular administration is to a joint selected from knee, elbow, hip, sternoclavicular, temporomandibular, carpal, tarsal, wrist, ankle, intervertebral disk, or ligamentum flavum.

100. The method of claim 92, wherein the hydrogel of the drug-delivery system is formed in-situ at a treatment site of a patient.

101. The method of claim 92, wherein the polymer network comprises or is composed of multi-arm units each having a core unit X and >3 arms, preferably in the range of 3 to 10, or 4 to 8, or 4 to 6 arms, attached to the core unit, wherein a crosslink formed by click chemistry reaction is located at the terminus of each arm linking it with another arm attached to another core of a multi-arm unit.

102. The method of claim 92, wherein the polymer network comprises first, second, and optionally more multi-arm units, wherein the crosslinks formed by click chemistry reactions are between the arms of the first multi-arm units and the second or more multi-arm units, wherein multi-arm units have the same or a different amount of arms and/or have the same or a different core unit X.

103. The method of claim 102, wherein at least one of the multi-arm units is a polymer multi-arm unit having polymer arms comprising polymer units, wherein the polymer multi-arm units have an average molecular weight of 1,000 to 100,000 Daltons.

104. The method of claim 92, wherein the polymer units are selected from at least one of polyethylene glycol (PEG) units, polypropylene glycol (PPG) units, polyglycolic acid (PGA), polylactic acid (PLA), or polylactic-co-glycolic acid (PLGA), random or block copolymers and/or combinations or mixtures of any of these, preferably polyethylene glycol units.

105. The method of claim 92, wherein each of the polymer units have an average molecular weight (Mn) in the range from about 1,000 to about 100,000 Daltons, or from about 2,000 to about 50,000 Daltons, or from about 3,000 to about 30,000 Daltons, wherein the average molecular weight (Mn) of the arms of the first multi-arm unit and the arms of the second multi-arm unit are the same or different.

106. The method of claim 101, wherein the hydrolysable ester bond is part of a linker unit L being located between the polymer unit terminus of an arm of a multi-arm unit and the crosslinks formed by click chemistry reactions.

107. The method of claim 106, wherein the linker unit L comprises a structure represented by Formula (i): 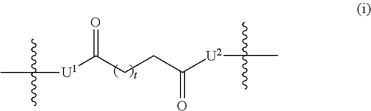
wherein U¹ and U² are independently NH or O and can be the same or different, wherein at least one, preferably both, of U¹ and U² is O, and wherein t is an integer from 0 to 10, such as a diacid, such as succinic, glutaric, adipic, or azelaic diacids.

108. The method of claim 92, wherein the polymer network comprises multi-arm units represented by formula (ii): 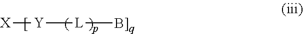
wherein X is the core unit of a multi-arm unit, Y is a polymeric unit, L is a linker unit comprising the hydrolysable ester bond, A is a crosslink formed by a click chemistry reaction, p is either 0 or 1, and q is an integer from 3 to 10.

109. The method of claim 92, wherein the crosslinks are formed by a click chemistry reaction such as a copper-free SPAAC or IEDDA reaction, wherein a first functional group of a first multi-arm precursor or unit is reacted with at least one complementary, second functional group of a second multi-arm precursor or unit, wherein the second, complementary functional group is capable of reacting with the first functional group in a click chemistry reaction, the first and second functional groups being selected from the pairs comprising an alkyne moiety such as a dibenzocyclooctyne (DBCO) or a bicyclo[6.1.0]nonyne (BCN), and an azide; or a norbornene or trans-cyclooctene (TCO); and a tetrazine (Tz) moiety.

110. A hydrogel for sustained drug-delivery, the hydrogel including an active agent, the hydrogel comprising an interpenetrating covalently crosslinked dual polymer network and containing the at least one active agent, wherein the dual polymer network comprises
a) a plurality of polymer units,
b) chemical bonds that can be cleaved by hydrolysis, wherein at least one of the hydrolysable bonds is an ester bond, and
c) crosslinks formed by click chemistry reactions between first and second multi-arm precursors bearing functional groups reactive by click chemistry, and
d) crosslinks formed by electrophile-nucleophile reactions between third and fourth multi-arm precursors bearing functional groups reactive by electrophile-nucleophile reactions.

111. A kit suitable for use for in-situ forming a hydrogel for sustained drug-delivery including an active agent as defined in claim 110, the kit comprising in separate containers:
a) a first container including a first precursor formulation including at least one first multi-arm precursor having first functional groups suitable for forming a connection by click chemistry with complementary second functional groups, and optionally at least one solvent;
b) a second container including a second precursor formulation including at least one second multi-arm precursor having second functional groups suitable for forming a connection by click chemistry with the first functional groups, and optionally at least one solvent; and
c) at least one active agent, included in a separate, third container and/or included in at least one, or both of the first and second containers, and optionally at least one solvent;
d) wherein at least one of the first multi-arm precursor and/or the at least one second multi-arm precursor is a polymer multi-arm precursor having polymer arms comprising polymer units, and wherein at least one of the first multi-arm precursors or the second multi-arm precursors includes a linker unit L comprising a hydrolysable ester bond.
